(19) United States
(12) Patent Application Publication
(10) Pub. No.: US 2026/0335462 A1
(43) Pub. Date: Oct. 29, 2026

(54) COMPOSITION OF LIPID NANOPARTICLES

(71) Applicant: SUZHOU ABOGEN BIOSCIENCES CO., LTD., Suzhou Jiangsu (CN)

(72) Inventors: Yuhang JIANG, Suzhou Jiangsu (CN); Bo YING, Jiangsu (CN)

(21) Appl. No.: 19/104,621

(22) PCT Filed: Aug. 17, 2023
(86) PCT No.: PCT/CN2023/113447

§ 371 Date: Feb. 18, 2025

(30) Foreign Application Priority Data
Aug. 18, 2022 (WO) PCT/CN2022/113414

(51) Int. Cl. A61K 47/69; A61K 9/51; A61K 31/7105; A61K 48/00

(52) U.S. Cl. CPC A61K 47/6929; A61K 9/5123; A61K 31/7105; A61K 48/0041

(57) ABSTRACT

Provided herein are lipid nanoparticle compositions comprising a cationic lipid, a steroid, a polymer conjugated lipid, and no more than 0.5 mole percent phospholipid, that can be used for delivery of therapeutic payloads (e.g., mRNA, siRNA, DNA) for therapeutic or prophylactic purposes.

### 1. CROSS-REFERENCE TO RELATED APPLICATIONS

This application claims priority to International Patent Application No. PCT/CN2022/113414, filed on Aug. 18, 2022, the entirety of which is incorporated herein by reference.

### 2. FIELD

Provided herein are lipid nanoparticle (LNP) compositions comprising a cationic lipid, a steroid, a polymer conjugated lipid, and no more than 0.5 mole percent phospholipid, that can be used for delivery of therapeutic payloads (e.g., mRNA, siRNA, miRNA, non-coding RNA, DNA, antisense oligonucleotides, gene editing cassettes) for therapeutic or prophylactic purposes.

### 3. BACKGROUND

Therapeutic nucleic acids have tremendous potential to revolutionize vaccination, gene therapies, cell therapies, and treatments of cancers and genetic diseases. Since the commencement of the first clinical studies on therapeutic nucleic acids in the 2000s, significant progresses have been made through the design, modification, and delivery of nucleic acid molecules. A variety of nucleic acid-based therapies have been studied, including mRNA, siRNA, plasmid DNA, viral and bacterial vectors and patient derived cellular therapies, over a dozen of which have been approved by US Food and Drug Administration (FDA) for use in humans and many more are in clinic trials. However, nucleic acid therapeutics still face multiple challenges, such as off-target effects, un-wanted immune stimulation, and low delivery efficiency. For mRNA-based therapies, rapid degradation of mRNA by nuclease in serum makes the delivery process more challenging.

Current LNP delivery system typically comprises a cationic lipid, a phospholipid, cholesterol, and a PEG-lipid conjugate. Four-lipid-component LNP formulations (e.g., with a typical lipid ratio of cationic lipid:cholesterol:DSPC:DMG-PEG=50:38.5:10:1.5) have been proved successful in multiple commercial drug products, yet there is still room for improvement. There remains a need to develop simplified lipid nanoparticle compositions without sacrificing the delivery efficiency of nucleic acid payloads.

### 4. SUMMARY

In one embodiment, provided herein is a lipid nanoparticle that comprises a reduced amount (e.g., as compared to the conventional four-lipid-component lipid nanoparticle) of a phospholipid component.

In one embodiment, provided herein is a lipid nanoparticle comprising:
(a) a cationic lipid at the amount of from about 40 mol % to about 75 mol % of the total lipid present in the nanoparticle, wherein the cationic lipid comprises (i) at least one tertiary amine or quaternary ammonium moiety, and (ii) at least one C₁₄-C₄₂ alkyl ester moiety;
(b) a steroid at the amount of from about 22 mol % to about 59.5 mol % of the total lipid present in the nanoparticle;
(c) a polymer conjugated lipid at the amount of from about 0.5 mol % to about 2.5 mol % of the total lipid present in the nanoparticle; and
(d) a nucleic acid;
   provided that the lipid nanoparticle comprises no more than 0.5 mole percent phospholipid.

In one embodiment, the cationic lipid is of Formula (I):

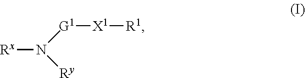

or a pharmaceutically acceptable salt, prodrug or stereoisomer thereof, wherein G¹, X¹, R¹, R^{x} and R^{y} are as defined herein or elsewhere.

In one embodiment, the cationic lipid is of Formula (III):

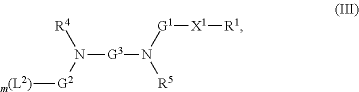

or a pharmaceutically acceptable salt, prodrug or stereoisomer thereof, wherein G¹, X¹, R¹, R^{x} and R^{y} are as defined herein or elsewhere.

In one embodiment, a three-lipid-component lipid nanoparticle provided herein (with no phospholipid component, or reduced/insignificant amount of phospholipid component) has an increased size, as compared to the conventional four-lipid-component lipid nanoparticle, such as a four-lipid-component lipid nanoparticle wherein the molar ratio of the cationic lipid:the steroid:the phospholipid:the polymer conjugated lipid is about 50:38.5:10:1.5. In one embodiment, the size of a three-lipid-component nanoparticle provided herein is from about 110 nm to about 130 nm.

Also provided herein are pharmaceutical compositions comprising the lipid nanoparticles provided herein, and a pharmaceutically acceptable excipient.

Also provided herein are methods of using the lipid nanoparticles provided herein or the pharmaceutical compositions provided herein for expressing an mRNA in a mammalian cell or a tissue of a mammal, for introducing an mRNA that encodes a protein into a cell, for treating a disease or disorder in a human, or for treating a disease or disorder caused by impaired expression of a protein in a human.

### 5. BRIEF DESCRIPTION OF THE FIGURES

FIG. 1 illustrates the in vivo protein (hEPO) expression 6 h after intravenous administration of LNPs containing Lipid-5. The LNP compositions and properties are listed in Table 1 and Table 5. All expression data are normalized to Formulation L-0. Error bars are shown to represent the Standard Error of Mean (S.E.M.).

FIG. 2 illustrates the in vivo protein (hEPO) expression 6 h after intravenous administration of LNPs containing SM-102. The LNP compositions and properties are listed in Table 2 and Table 6. All expression data are normalized to Formulation S-0. Error bars are shown to represent the S.E.M.

FIG. 3 illustrates the in vivo protein (hEPO) expression 6 h after intravenous administration of LNPs containing C1 or ALC-0315. The LNP compositions and properties are listed in Tables 3, 4, 7 and 8. All expression data are normalized to Formulation A-0 or C1-0. Error bars are shown to represent the S.E.M.

FIG. 4 illustrates the in vivo protein (hEPO) expression 6 h after intravenous administration of LNPs containing more than 3 mol % of PEG-lipid. All expression data are normalized to Formulation L-0 or S-0. Error bars are shown to represent the S.E.M.

FIG. 5A, FIG. 5B, and FIG. 5C show the long-term stability of certain Lipid-5 containing LNPs stored at room temperature, 2-8° C., and −20° C., respectively, over the course of 4 months by evaluating the purity of encapsulated mRNA with Fragment Analysis (FA) assay.

FIG. 5D and FIG. 5E show the updated long-term stability of certain Lipid-5 containing LNPs stored at 2-8° C. or −20° C., respectively, for up to 400 days.

FIG. 6A shows in vivo GFP expression 6 h after intramuscular administration of certain Lipid-5 containing LNPs; FIG. 6B shows normalized IL-6 level by the amount of expressed GFP.

FIG. 7A and FIG. 7B show the mice body weight change monitored over 7 days after intravenous administration and intramuscular administration, respectively, of certain Lipid-5 containing LNPs.

FIG. 8 illustrates the in vivo protein (hEPO) expression 6 h after intravenous administration of LNPs containing C2. The LNP compositions and properties are listed in Table 9 and Table 10. All expression data are normalized to Formulation C2-0. Error bars are shown to represent the S.E.M.

FIG. 9A and FIG. 9B show Cyro-TEM images of LNP composed C1/Cholesterol/DMG-PEG (60/38.5/1.5%) and LNP composed C1/Cholesterol/DMG-PEG (65/33/2.0%), respectively.

### 6. DETAILED DESCRIPTION

### 6.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003).

### 6.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein and unless otherwise specified, the term “lipid” refers to a group of organic compounds that include, but are not limited to, esters of fatty acids and are generally characterized by being poorly soluble in water, but soluble in many nonpolar organic solvents, such as pentane, hexane, dichloromethane, chloroform, ethyl acetate and diethyl ether. While lipids generally have poor solubility in water, there are certain categories of lipids (e.g., lipids modified by polar groups, e.g., DMG-PEG2000) that have limited aqueous solubility and can dissolve in water under certain conditions. Known types of lipids include biological molecules such as fatty acids, waxes, sterols, fat-soluble vitamins, monoglycerides, diglycerides, triglycerides, and phospholipids. Lipids can be divided into at least three classes: (1) “simple lipids,” which include fats and oils as well as waxes; (2) “compound lipids,” which include phospholipids and glycolipids (e.g., DMPE-PEG2000); and (3) “derived lipids” such as steroids. Further, as used herein, lipids also encompass synthetic lipidoid compounds. The term “lipidoid compound,” also simply “lipidoid”, refers to a lipid-like compound (e.g. an amphiphilic compound with lipid-like physical properties).

The term “lipid nanoparticle” or “LNP” refers to a particle having at least one dimension on the order of nanometers (nm) (e.g., 1 to 1,000 nm), which contains one or more types of lipid molecules. The LNP provided herein can further contain at least one non-lipid payload molecule (e.g., one or more nucleic acid molecules). In one embodiment, the LNP comprises a non-lipid payload molecule either partially or completely encapsulated inside a lipid shell. Particularly, in one embodiment, wherein the payload is a negatively charged molecule such as nucleic acids that include RNA and DNA, and the lipid components of the LNP comprise at least one cationic lipid. Without being bound by the theory, it is contemplated that the cationic lipids can interact with the negatively charged payload molecules by electrostatic effects and facilitates incorporation and/or encapsulation of the payload into the LNP during LNP formation. Other lipids that can form part of a LNP as provided herein include but are not limited to neutral lipids and charged lipids, such as steroids, polymer conjugated lipids, and various zwitterionic lipids. In certain embodiments, an LNP according to the present disclosure comprises one or more cationic lipids of Formula (I) and (II) as described.

The term “cationic lipid” refers to a lipid that is either positively charged at any pH value or hydrogen ion activity of its environment, or capable of being positively charged in response to the pH value or hydrogen ion activity of its environment (e.g., the environment of its intended use). Thus, the term “cationic lipid” encompasses “permanently cationic lipid”, “cationisable lipid”, and “ionizable lipid”. In certain embodiments, the positive charge in a cationic lipid results from the presence of a quaternary nitrogen atom. In certain embodiments, the cationic lipid comprises a zwitterionic lipid that assumes a positive charge in the environment of its intended use (e.g., at physiological pH). In certain embodiments, the cationic lipid is an ionizable lipid. In certain embodiments, the cationic lipid is one or more lipids of Formula (I) or (II) as described herein.

The term “polymer conjugated lipid” refers to a molecule comprising both a lipid portion and a polymer portion. An example of a polymer conjugated lipid is a pegylated lipid (PEG-lipid), in which the polymer portion comprises a polyethylene glycol.

As used herein, and unless otherwise specified, the term “alkyl” refers to a straight or branched hydrocarbon chain radical consisting solely of carbon and hydrogen atoms, which is saturated. An alkyl does not contain any carbon-carbon double bond or carbon-carbon triple bond. In one embodiment, the alkyl group has, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₅ alkyl), one to six carbon atoms (C₁-C₆ alkyl), two to twelve carbon atoms (C₁-C₁₂ alkyl), fourteen to forty-two carbon atoms (C₁₄-C₄₂ alkyl), one to six carbon atoms (C₁-C₆ alkyl), six to forty-two carbon atoms (C₆-C₄₂ alkyl), six to twenty-four carbon atoms (C₆-C₂₄ alkyl), or fourteen to twenty-four carbon atoms (C₁₄-C₂₄ alkyl) and which is attached to the rest of the molecule by a single bond. As non-limiting examples, alkyl groups include methyl, ethyl, n-propyl, 1-methylethyl (isopropyl), n-butyl, n-pentyl, 1,1-dimethylethyl (t-butyl), 3-methylhexyl, 2-methylhexyl and the like. Unless otherwise specified, an alkyl group is optionally substituted.

As used herein, and unless otherwise specified, the term “alkylene” or “alkylene chain” refers to a straight or branched multivalent (e.g., divalent or trivalent) hydrocarbon chain linking the rest of the molecule to a radical group consisting solely of carbon and hydrogen and is saturated. In one embodiment, the alkylene has, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₈ alkylene), one to six carbon atoms (C₁-C₆ alkylene), two to four carbon atoms (C₂-C₄ alkylene), one to two carbon atoms (C₁-C₂ alkylene) or two to twelve carbon atoms (C₂-C₁₂ alkylene). As non-limiting examples, alkylene groups include methylene, ethylene, propylene, iso-propylene, n-butylene, t-butylene and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless otherwise specified, an alkylene chain is optionally substituted.

As used herein, and unless otherwise specified, the term “cycloalkyl” refers to a non-aromatic monocyclic or polycyclic hydrocarbon radical consisting solely of carbon and hydrogen atoms and is saturated. Cycloalkyl group may include fused or bridged ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl) or from 3 to 8 ring carbon atoms (C₃-C₈ cycloalkyl). The cycloalkyl is attached to the rest of the molecule by a single bond. As non-limiting examples, monocyclic cycloalkyl radicals include cyclopropyl, cyclobutyl, cyclopentyl, cyclohexyl, cycloheptyl, and cyclooctyl. Examples of polycyclic cycloalkyl radicals include adamantyl, norbornyl, decalinyl, 7,7-dimethyl-bicyclo[2.2.1]heptanyl, and the like. Unless otherwise specified, a cycloalkyl group is optionally substituted.

As used herein, and unless otherwise specified, the term “cycloalkenyl” refers to a non-aromatic monocyclic or polycyclic hydrocarbon radical consisting solely of carbon and hydrogen atoms, and which includes one or more carbon-carbon double bonds. Cycloalkenyl may include fused or bridged ring systems. In one embodiment, the cycloalkenyl has, for example, from 3 to 15 ring carbon atoms (C₃-C₁₅ cycloalkenyl), from 3 to 10 ring carbon atoms (C₃-C₁₀ cycloalkenyl), or from 3 to 8 ring carbon atoms (C₃-C₈ cycloalkenyl). The cycloalkenyl is attached to the rest of the molecule by a single bond. As non-limiting examples, cycloalkenyl radicals include cyclopropenyl, cyclobutenyl, cyclopentenyl, cyclohexenyl, cycloheptenyl, cyclooctenyl, and the like. Unless otherwise specified, a cycloalkenyl group is optionally substituted.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 18 ring carbon atoms (C₆-C₁₈ aryl), from 6 to 14 ring carbon atoms (C₆-C₁₄ aryl), or from 6 to 10 ring carbon atoms (C₆-C₁₀ aryl). Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). Unless otherwise specified, an aryl group is optionally substituted.

When the groups described herein are said to be “substituted,” they may be substituted with any appropriate substituent or substituents. Illustrative examples of substituents include, but are not limited to, those found in the exemplary compounds and embodiments provided herein, as well as: a halogen atom such as F, Cl, Br, or I; cyano; oxo (═O); hydroxyl (—OH); alkyl; alkenyl; alkynyl; cycloalkyl; aryl; —(C═O)OR′; —O(C═O)R′; —C(═O)R′; —OR′; —S(O)ₓR′; —S—SR′; —C(═O)SR′; —SC(═O)R′; —NR′R′; —NR′C(═O)R′; —C(═O)NR′R′; —NR′C(═O)NR′R′; —OC(═O)NR′R′; —NR′C(═O)OR′; —NR'S(O)ₓNR′R′; —NR'S(O)XR′; and —S(O)ₓNR′R′, wherein: R′ is, at each occurrence, independently H, C₁-C₁₅ alkyl or cycloalkyl, and x is 0, 1 or 2. In one embodiment, the substituent is a C₁-C₁₂ alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group (—OR′). In other embodiments, the substituent is a carboxyl group. In other embodiments, the substituent is an amino group (—NR′R′).

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

As used herein, and unless otherwise specified, the term “pharmaceutically acceptable salt” includes both acid and base addition salts.

As non-limiting examples, pharmaceutically acceptable acid addition salts include hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

As non-limiting examples, pharmaceutically acceptable base addition salt include salts prepared from addition of an inorganic base or an organic base to a free acid compound. Salts derived from inorganic bases include, but are not limited to, the sodium, potassium, lithium, ammonium, calcium, magnesium, iron, zinc, copper, manganese, aluminum salts and the like. In one embodiment, the inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In one embodiment, the organic bases are isopropylamine, diethylamine, ethanolamine, trimethylamine, dicyclohexylamine, choline and caffeine.

A compound provided herein may contain one or more asymmetric centers and may thus give rise to enantiomers, diastereomers, and other stereoisomeric forms that may be defined, in terms of absolute stereochemistry, such as (R)- or (S)- or, as (D)- or (L)- for amino acids. Unless otherwise specified, a compound provided herein is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution.

It should also be noted a compound described herein can contain unnatural proportions of atomic isotopes at one or more of the atoms. For example, the compounds may be radiolabeled with radioactive isotopes, such as for example, tritium (³H), carbon-14 (¹⁴C), carbon-11 (¹¹C), fluorine-18 (¹⁸C), sulfur-35 (³⁵S), iodine-125 (¹²⁵I), or may be isotopically enriched, such as with deuterium (²H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of a compound as described herein, whether radioactive or not, are intended to be encompassed within the scope of the embodiments provided herein. In one embodiment, there are provided isotopologs of a compound described herein, for example, the isotopologs are deuterium, carbon-13, and/or nitrogen-15 enriched. As used herein, “deuterated”, means a compound wherein at least one hydrogen (H) has been replaced by deuterium (indicated by D or 2H), that is, the compound is enriched in deuterium in at least one position.

It should be noted that if there is a discrepancy between a depicted structure and a name for that structure, the depicted structure is to be accorded more weight.

As used herein, and unless otherwise specified, the term “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “composition” is intended to encompass a product containing the specified ingredients (e.g., a mRNA molecule provided herein) in, optionally, the specified amounts.

The term “polynucleotide” or “nucleic acid,” as used interchangeably herein, refers to polymers of nucleotides of any length and includes, e.g., DNA and RNA. The nucleic acid can be deoxyribonucleotides, ribonucleotides, modified nucleotides or bases, and/or their analogs, or any substrate that can be incorporated into a polymer by DNA or RNA polymerase or by a synthetic reaction. A nucleic acid may comprise modified nucleotides, such as methylated nucleotides and their analogs. Nucleic acid can be in either single- or double-stranded forms. As used herein and unless otherwise specified, “nucleic acid” also includes nucleic acid mimics such as locked nucleic acids (LNAs), peptide nucleic acids (PNAs), and morpholinos. “Oligonucleotide,” as used herein, refers to short synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “non-naturally occurring” when used in reference to a nucleic acid molecule as described herein is intended to mean that the nucleic acid molecule is not found in nature. A non-naturally occurring nucleic acid encoding a viral peptide or protein contains at least one genetic alternation or chemical modification not normally found in a naturally occurring strain of the virus, including wild-type strains of the virus. Genetic alterations include, for example, modifications introducing expressible nucleic acid sequences encoding peptides or polypeptides heterologous to the virus, other nucleic acid additions, nucleic acid deletions, nucleic acid substitution, and/or other functional disruption of the virus' genetic material. Such modifications include, for example, modifications in the coding regions and functional fragments thereof, for heterologous, homologous or both heterologous and homologous polypeptides for the viral species. Additional modifications include, for example, modifications in non-coding regulatory regions in which the modifications alter expression of a gene or operon. Additional modifications also include, for example, incorporation of a nucleic acid sequence into a vector, such as a plasmid or an artificial chromosome. Chemical modifications include, for example, one or more functional nucleotide analog as described herein.

An “isolated nucleic acid” is a nucleic acid, for example, an RNA, DNA, or a mixture of nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as an mRNA molecule, can be substantially free of other cellular material, or culture medium when produced by recombinant techniques, or substantially free of chemical precursors or other chemicals when chemically synthesized. In a specific embodiment, one or more nucleic acid molecules encoding an antigen as described herein are isolated or purified. The term embraces nucleic acid sequences that have been removed from their naturally occurring environment and includes recombinant or cloned DNA or RNA isolates and chemically synthesized analogues or analogues biologically synthesized by heterologous systems. A substantially pure molecule may include isolated forms of the molecule.

The term “encoding nucleic acid” or grammatical equivalents thereof as it is used in reference to nucleic acid molecule encompasses (a) a nucleic acid molecule in its native state or when manipulated by methods well known to those skilled in the art that can be transcribed to produce mRNA which is then translated into a peptide and/or polypeptide, and (b) the mRNA molecule itself. The antisense strand is the complement of such a nucleic acid molecule, and the encoding sequence can be deduced therefrom. The term “coding region” refers to a portion in an encoding nucleic acid sequence that is translated into a peptide or polypeptide. The term “untranslated region” or “UTR” refers to the portion of an encoding nucleic acid that is not translated into a peptide or polypeptide. Depending on the orientation of a UTR with respect to the coding region of a nucleic acid molecule, a UTR is referred to as the 5′-UTR if located to the 5′-end of a coding region, and a UTR is referred to as the 3′-UTR if located to the 3′-end of a coding region.

The term “mRNA” as used herein refers to a message RNA molecule comprising one or more open reading frame (ORF) that can be translated by a cell or an organism provided with the mRNA to produce one or more peptide or protein product. The region containing the one or more ORFs is referred to as the coding region of the mRNA molecule. In certain embodiments, the mRNA molecule further comprises one or more untranslated regions (UTRs).

In certain embodiments, the mRNA is a monocistronic mRNA that comprises only one ORF. In certain embodiments, the monocistronic mRNA encodes a peptide or protein comprising at least one epitope of a selected antigen (e.g., a pathogenic antigen or a tumor associated antigen). In other embodiments, the mRNA is a multicistronic mRNA that comprises two or more ORFs. In certain embodiments, the multiecistronic mRNA encodes two or more peptides or proteins that can be the same or different from each other. In certain embodiments, each peptide or protein encoded by a multicistronic mRNA comprises at least one epitope of a selected antigen. In certain embodiments, different peptide or protein encoded by a multicistronic mRNA each comprises at least one epitope of different antigens. In any of the embodiments described herein, the at least one epitope can be at least 2, at least 3, at least 4, at least 5, at least 6, at least 7, at least 8, at least 9, or at least 10 epitopes of an antigen.

The term “nucleobases” encompasses purines and pyrimidines, including natural compounds adenine, thymine, guanine, cytosine, uracil, inosine, and natural or synthetic analogs or derivatives thereof.

The term “functional nucleotide analog” as used herein refers to a modified version of a canonical nucleotide A, G, C, U or T that (a) retains the base-pairing properties of the corresponding canonical nucleotide, and (b) contains at least one chemical modification to (i) the nucleobase, (ii) the sugar group, (iii) the phosphate group, or (iv) any combinations of (i) to (iii), of the corresponding natural nucleotide. As used herein, base pairing encompasses not only the canonical Watson-Crick adenine-thymine, adenine-uracil, or guanine-cytosine base pairs, but also base pairs formed between canonical nucleotides and functional nucleotide analogs or between a pair of functional nucleotide analogs, wherein the arrangement of hydrogen bond donors and hydrogen bond acceptors permits hydrogen bonding between a modified nucleobase and a canonical nucleobase or between two complementary modified nucleobase structures. For example, a functional analog of guanosine (G) retains the ability to base-pair with cytosine (C) or a functional analog of cytosine. One example of such non-canonical base pairing is the base pairing between the modified nucleotide inosine and adenine, cytosine, or uracil. As described herein, a functional nucleotide analog can be either naturally occurring or non-naturally occurring. Accordingly, a nucleic acid molecule containing a functional nucleotide analog can have at least one modified nucleobase, sugar group and/or internucleoside linkage. Exemplary chemical modifications to the nucleobases, sugar groups, or internucleoside linkages of a nucleic acid molecule are provided herein.

The terms “translational enhancer element,” “TEE” and “translational enhancers” as used herein refers to an region in a nucleic acid molecule that functions to promotes translation of a coding sequence of the nucleic acid into a protein or peptide product, such as via cap-dependent or cap-independent translation. A TEE typically locates in the UTR region of a nucleic acid molecule (e.g., mRNA) and enhance the translational level of a coding sequence located either upstream or downstream. For example, a TEE in a 5′-UTR of a nucleic acid molecule can locate between the promoter and the starting codon of the nucleic acid molecule. Various TEE sequences are known in the art (Wellensiek et al. Genome-wide profiling of human cap-independent translation-enhancing elements, Nature Methods, 2013 August; 10(8): 747-750; Chappell et al. PNAS Jun. 29, 2004 101 (26) 9590-9594). Some TEEs are known to be conserved across multiple species (Pánek et al. Nucleic Acids Research, Volume 41, Issue 16, 1 Sep. 2013, Pages 7625-7634).

The term “siRNA” as used herein refers to a “small interfering” or “short interfering RNA”. The siRNA is a RNA duplex of nucleotides that can be targeted to a gene of interest. A “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In one embodiment, the length of the duplex of siRNAs is less than 30 nucleotides. In one embodiment, the length of the duplex of siRNAs is between 20 to 25 nucleotides. In one embodiment, the duplex of siRNA can be 29, 28, 27, 26, 25, 24, 23, 22, 21, 20, 18, 16, 14, 12 or 10 nucleotides in length. The RNA duplex portion of the siRNA can be part of a hairpin structure. In addition to the duplex portion, the hairpin structure may contain a loop portion positioned between the two sequences that form the duplex. The loop can vary in length. In one embodiment the loop is 5, 6, 7, 8, 9, 10, 11, 12 or 13 nucleotides in length. The hairpin structure can also contain 3′ or 5′ overhang portions. In one embodiment, the overhang is a 3′ or a 5′ overhang 0, 1, 2, 3, 4 or 5 nucleotides in length. The siRNA can be encoded by a nucleic acid sequence, and the nucleic acid sequence can also include a promoter. The nucleic acid sequence can also include a polyadenylation signal. In one embodiment, the polyadenylation signal is a synthetic minimal polyadenylation signal. In one embodiment, siRNA is used for “gene silencing”. “Gene silencing” refers to the suppression of gene expression. Gene silencing may be mediated through processes that affect transcription and/or through processes that affect post-transcriptional mechanisms. In one embodiment, gene silencing occurs when siRNA initiates the degradation of the mRNA of a gene of interest in a sequence-specific manner via RNA interference. In one embodiment, siRNA interferes with the expression of specific genes with complementary nucleotide sequences by degrading mRNA after transcription, preventing translation. In one embodiment, gene silencing may be allele-specific, which refers to the specific silencing of one allele of a gene. In one embodiment, siRNA is used for gene “knock-down”. “Knock-down” refers to a technique of gene silencing in which the expression of a target gene is reduced as compared to the gene expression prior to the introduction of the siRNA, which can lead to the inhibition of production of the target gene product. The term “reduced” is used herein to indicate that the target gene expression is lowered by 1-100%. For example, the expression may be reduced by 10, 20, 30, 40, 50, 60, 70, 80, 90, 95, 97 or 99%. Knock-down of gene expression can be directed using siRNA.

The terms “polypeptide” and “protein” are used interchangeably herein to refer to a polymer of greater than fifty (50) amino acid residues linked by covalent peptide bonds. That is, a description directed to a polypeptide applies equally to a description of a protein, and vice versa. The terms apply to naturally occurring amino acid polymers as well as amino acid polymers in which one or more amino acid residues is a non-naturally occurring amino acid (e.g., an amino acid analog). As used herein, the terms encompass amino acid chains of any length, including full length proteins (e.g., antigens).

In the context of a peptide or polypeptide, the term “derivative” as used herein refers to a peptide or polypeptide that comprises an amino acid sequence of the viral peptide or protein, or a fragment of a viral peptide or protein, which has been altered by the introduction of amino acid residue substitutions, deletions, or additions. The term “derivative” as used herein also refers to a viral peptide or protein, or a fragment of a viral peptide or protein, which has been chemically modified, e.g., by the covalent attachment of any type of molecule to the polypeptide. For example, but not by way of limitation, a viral peptide or protein or a fragment of the viral peptide or protein may be chemically modified, e.g., by glycosylation, acetylation, pegylation, phosphorylation, amidation, derivatization by known protecting/blocking groups, proteolytic cleavage, chemical cleavage, formulation, metabolic synthesis of tunicamycin, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the viral peptide or protein. Further, a derivative of a viral peptide or protein or a fragment of a viral peptide or protein may contain one or more non-classical amino acids. In specific embodiments, a derivative is a functional derivative of the native or unmodified peptide or polypeptide from which it was derived.

The term “functional derivative” refers to a derivative that retains one or more functions or activities of the naturally occurring or starting peptide or polypeptide from which it was derived. For example, a functional derivative of a coronavirus S protein may retain the ability to bind one or more of its receptors on a host cell. For example, a functional derivative of a coronavirus N protein may retain the ability to bind RNA or the package viral genome.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, fragments refers to polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the amino acid sequence of a polypeptide. In a specific embodiment, a fragment of a polypeptide retains at least 1, at least 2, at least 3, or more functions of the polypeptide.

The term “immunogenic fragment” as used herein in the context of a peptide or polypeptide (e.g., a protein), refers to a fragment of a peptide or polypeptide that retains the ability of the peptide or polypeptide in eliciting an immune response upon contacting the immune system of a mammal, including innate immune responses and/or adaptive immune responses. In one embodiment, an immunogenic fragment of a peptide or polypeptide can be an epitope.

The term “antigen” refers to a substance that can be recognized by the immune system of a subject (including by the adaptive immune system), and is capable of triggering an immune response after the subject is contacted with the antigen (including an antigen-specific immune response). In certain embodiments, the antigen is a protein associated with a diseased cell, such as a cell infected by a pathogen or a neoplastic cell (e.g., tumor associated antigen (TAA)).

An “epitope” is the site on the surface of an antigen molecule to which a single antibody molecule binds, such as a localized region on the surface of an antigen that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. In certain embodiments, an epitope is a three-dimensional surface feature of a polypeptide. In other embodiments, an epitope is linear feature of a polypeptide. Generally an antigen has several or many different epitopes and may react with many different antibodies.

The term “genetic vaccine” as used herein refers to a therapeutic or prophylactic composition comprising at least one nucleic acid molecule encoding an antigen associated with a target disease (e.g., an infectious disease or a neoplastic disease). Administration of the vaccine to a subject (“vaccination”) allows for the production of the encoded peptide or protein, thereby eliciting an immune response against the target disease in the subject. In certain embodiments, the immune response comprises adaptive immune response, such as the production of antibodies against the encoded antigen, and/or activation and proliferations of immune cells capable of specifically eliminating diseased cells expressing the antigen. In certain embodiments, the immune response further comprises innate immune response. According to the present disclosure, a vaccine can be administered to a subject either before or after the onset of clinical symptoms of the target disease. In one embodiment, vaccination of a healthy or asymptomatic subject renders the vaccinated subject immune or less susceptible to the development of the target disease. In one embodiment, vaccination of a subject showing symptoms of the disease improves the condition of, or treats, the disease in the vaccinated subject.

The terms “innate immune response” and “innate immunity” are recognized in the art, and refer to non-specific defense mechanism a body's immune system initiates upon recognition of pathogen-associated molecular patterns, which involves different forms of cellular activities, including cytokine production and cell death through various pathways. As used herein, innate immune responses include, without limitation, increased production of inflammation cytokines (e.g., type I interferon or IL-10 production), activation of the NFκB pathway, increased proliferation, maturation, differentiation and/or survival of immune cells, and in some cases, induction of cell apoptosis. Activation of the innate immunity can be detected using methods known in the art, such as measuring the (NF)-κB activation.

The terms “adaptive immune response” and “adaptive immunity” are recognized in the art, and refer to antigen-specific defense mechanism a body's immune system initiates upon recognition of a specific antigen, which include both humoral response and cell-mediated responses. As used herein, adaptive immune responses include cellular responses that is triggered and/or augmented by a vaccine composition, such as a genetic composition described herein. In one embodiment, the vaccine composition comprises an antigen that is the target of the antigen-specific adaptive immune response. In other embodiments, the vaccine composition, upon administration, allows the production in an immunized subject of an antigen that is the target of the antigen-specific adaptive immune response. Activation of an adaptive immune response can be detected using methods known in the art, such as measuring the antigen-specific antibody production, or the level of antigen-specific cell-mediated cytotoxicity.

The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a polypeptide, a fragment or an epitope thereof. Antibodies also include, but are not limited to, synthetic antibodies, recombinantly produced antibodies, camelized antibodies, intrabodies, anti-idiotypic (anti-Id) antibodies, and functional fragments of any of the above, which refers to a portion of an antibody heavy or light chain polypeptide that retains some or all of the binding activity of the antibody from which the fragment was derived. Non-limiting examples of functional fragments include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “administer” or “administration” refers to the act of injecting or otherwise physically delivering a substance as it exists outside the body (e.g., a lipid nanoparticle composition as described herein) into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

“Chronic” administration refers to administration of the agent(s) in a continuous mode (e.g., for a period of time such as days, weeks, months, or years) as opposed to an acute mode, so as to maintain the initial therapeutic effect (activity) for an extended period of time. “Intermittent” administration is treatment that is not consecutively done without interruption, but rather is cyclic in nature.

The term “targeted delivery” or the verb form “target” as used herein refers to the process that promotes the arrival of a delivered agent (such as a therapeutic payload molecule in a lipid nanoparticle composition as described herein) at a specific organ, tissue, cell and/or intracellular compartment (referred to as the targeted location) more than any other organ, tissue, cell or intracellular compartment (referred to as the non-target location). Targeted delivery can be detected using methods known in the art, for example, by comparing the concentration of the delivered agent in a targeted cell population with the concentration of the delivered agent at a non-target cell population after systemic administration. In certain embodiments, targeted delivery results in at least 2 fold higher concentration at a targeted location as compared to a non-target location.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition, including, for example, infection and neoplasia. In one embodiment, the effective amount is a therapeutically effective amount or a prophylactically effective amount.

The term “therapeutically effective amount” as used herein refers to the amount of an agent (e.g., a vaccine composition) that is sufficient to reduce and/or ameliorate the severity and/or duration of a given disease, disorder, or condition, and/or a symptom related thereto (e.g., an infectious disease such as caused by viral infection, or a neoplastic disease such as cancer). A “therapeutically effective amount” of a substance/molecule/agent of the present disclosure (e.g., the lipid nanoparticle composition as described herein) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a lipid nanoparticle composition as described herein or a therapeutic or prophylactic agent contained therein (e.g., a therapeutic mRNA) effective to “treat” a disease, disorder, or condition, in a subject or mammal.

A “prophylactically effective amount” is an amount of a pharmaceutical composition that, when administered to a subject, will have the intended prophylactic effect, e.g., preventing, delaying, or reducing the likelihood of the onset (or reoccurrence) of a disease, disorder, condition, or associated symptom(s) (e.g., an infectious disease such as caused by viral infection, or a neoplastic disease such as cancer). Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose, and may occur only after administration of a series of doses. Thus, a therapeutically or prophylactically effective amount may be administered in one or more administrations.

As used herein and unless otherwise specified, the terms “treat”, “treating”, and “treatment” refer to an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise specified, the terms “prevent,” “preventing,” and “prevention” refer to reducing the likelihood of the onset (or recurrence) of a disease, disorder, condition, or associated symptom(s) (e.g., an infectious disease such as caused by viral infection, or a neoplastic disease such as cancer).

As used herein and unless otherwise specified, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as a lipid nanoparticle composition as described herein) to “manage” an infectious or neoplastic disease, one or more symptoms thereof, so as to prevent the progression or worsening of the disease.

The term “prophylactic agent” refers to any agent that can totally or partially inhibit the development, recurrence, onset, or spread of disease and/or symptom related thereto in a subject.

The term “therapeutic agent” refers to any agent that can be used in treating, preventing, or alleviating a disease, disorder, or condition, including in the treatment, prevention, or alleviation of one or more symptoms of a disease, disorder, or condition and/or a symptom related thereto.

The term “therapy” refers to any protocol, method, and/or agent that can be used in the prevention, management, treatment, and/or amelioration of a disease, disorder, or condition. In certain embodiments, the terms “therapies” and “therapy” refer to a biological therapy, supportive therapy, and/or other therapies useful in the prevention, management, treatment, and/or amelioration of a disease, disorder, or condition, known to one of skill in the art such as medical personnel.

As used herein, a “prophylactically effective serum titer” is the serum titer of an antibody in a subject (e.g., a human), that totally or partially inhibits the development, recurrence, onset, or spread of a disease, disorder, or condition, and/or symptom related thereto in the subject.

The term “side effects” encompasses unwanted and/or adverse effects of a therapy (e.g., a prophylactic or therapeutic agent). Unwanted effects are not necessarily adverse. An adverse effect from a therapy (e.g., a prophylactic or therapeutic agent) might be harmful, uncomfortable, or risky. Examples of side effects include, diarrhea, cough, gastroenteritis, wheezing, nausea, vomiting, anorexia, abdominal cramping, fever, pain, loss of body weight, dehydration, alopecia, dyspenea, insomnia, dizziness, mucositis, nerve and muscle effects, fatigue, dry mouth, loss of appetite, rashes or swellings at the site of administration, flu-like symptoms such as fever, chills, and fatigue, digestive tract problems, and allergic reactions. Additional undesired effects experienced by patients are numerous and known in the art. Many are described in Physician's Desk Reference (68th ed. 2014).

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal (e.g., a human) having an infectious disease or neoplastic disease. In another embodiment, the subject is a mammal (e.g., a human) at risk of developing an infectious disease or neoplastic disease.

“Substantially all” refers to at least about 60%, at least about 65%, at least about 70%, at least about 75%, at least about 80%, at least about 85%, at least about 90%, at least about 95%, at least about 98%, at least about 99%, or about 100%.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, 0.05%, or less of a given value or range.

The singular terms “a,” “an,” and “the” as used herein include the plural reference unless the context clearly indicates otherwise.

All publications, patent applications, accession numbers, and other references cited in this specification are herein incorporated by reference in their entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which may need to be independently confirmed.

A number of embodiments of the invention have been described. Nevertheless, it will be understood that various modifications may be made without departing from the spirit and scope of the invention. Accordingly, the descriptions in the experimental section and examples are intended to illustrate but not limit the scope of invention described in the claims.

### 6.3 Lipid Nanoparticle (LNP) Compositions

The phospholipid, generally recognized as a helper lipid or structure lipid in LNP, plays an important role in conventional LNP formulation. It is believed that the phospholipid confers structurally stability and promotes gene delivery efficiency in vivo by mediating membrane fusion. The three commercial LNP based products (Onpattro®, Comirnaty®, and mRNA-1273) all contain DSPC as a fundamental component (Eygeris et al. Chemistry of Lipid Nanoparticles for RNA Delivery. Acc. Chem. Res. 2022, 55, 2-12). However, as provided herein, it is shown that the phospholipid is not as essential as generally believed in LNP formulation.

In one embodiment, provided herein is a lipid nanoparticle that comprises a reduced amount (e.g., as compared to the conventional four-lipid-component lipid nanoparticle) of a phospholipid component. In one embodiment, the LNP comprises no more than 0.5 mole percent of phospholipid.

In one embodiment, provided herein is a lipid nanoparticle comprising:
(a) a cationic lipid at the amount of from about 40 mol % to about 75 mol % of the total lipid present in the nanoparticle, wherein the cationic lipid comprises (i) at least one tertiary amine or quaternary ammonium moiety, and (ii) at least one C₁₄-C₄₂ alkyl ester moiety;
(b) a steroid at the amount of from about 22 mol % to about 59.5 mol % of the total lipid present in the nanoparticle;
(c) a polymer conjugated lipid at the amount of from about 0.5 mol % to about 2.5 mol % of the total lipid present in the nanoparticle; and
(d) a nucleic acid;
   provided that the lipid nanoparticle comprises no more than 0.5 mole percent phospholipid.

In one embodiment, the amount of the cationic lipid is from about 40 mol % to about 75 mol % of the total lipid present in the nanoparticle. In one embodiment, the amount of the cationic lipid is from about 40 mol % to about 55 mol %. In one embodiment, the amount of the cationic lipid is from about 45 mol % to about 60 mol %. In one embodiment, the amount of the cationic lipid is from about 50 mol % to about 65 mol %. In one embodiment, the amount of the cationic lipid is from about 55 mol % to about 70 mol %. In one embodiment, the amount of the cationic lipid is from about 60 mol % to about 75 mol %. In one embodiment, the amount of the cationic lipid is from about 40 mol % to about 50 mol %. In one embodiment, the amount of the cationic lipid is from about 45 mol % to about 55 mol %. In one embodiment, the amount of the cationic lipid is from about 50 mol % to about 60 mol %. In one embodiment, the amount of the cationic lipid is from about 55 mol % to about 65 mol %. In one embodiment, the amount of the cationic lipid is from about 60 mol % to about 70 mol %. In one embodiment, the amount of the cationic lipid is from about 65 mol % to about 75 mol %. In one embodiment, the amount of the cationic lipid is from about 40 mol % to about 45 mol %. In one embodiment, the amount of the cationic lipid is from about 45 mol % to about 50 mol %. In one embodiment, the amount of the cationic lipid is from about 50 mol % to about 55 mol %. In one embodiment, the amount of the cationic lipid is from about 55 mol % to about 60 mol %. In one embodiment, the amount of the cationic lipid is from about 60 mol % to about 65 mol %. In one embodiment, the amount of the cationic lipid is from about 65 mol % to about 70 mol %. In one embodiment, the amount of the cationic lipid is from about 70 mol % to about 75 mol %.

In one embodiment, the amount of the cationic lipid is about 40 mol %, about 42.5 mol %, about 45 mol %, about 47.5 mol %, about 50 mol %, about 51 mol %, about 52 mol %, about 53 mol %, about 54 mol %, about 55 mol %, about 55.5 mol %, about 56 mol %, about 56.5 mol %, about 57 mol %, about 57.5 mol %, about 58 mol %, about 58.5 mol %, about 59 mol %, about 59.5 mol %, about 60 mol %, about 60.5 mol %, about 61 mol %, about 61.5 mol %, about 62 mol %, about 62.5 mol %, about 63 mol %, about 63.5 mol %, about 64 mol %, about 64.5 mol %, about 65 mol %, about 65.5 mol %, about 66 mol %, about 66.5 mol %, about 67 mol %, about 67.5 mol %, about 68 mol %, about 68.5 mol %, about 69 mol %, about 70 mol %, about 72.5 mol %, or about 75 mol % of the total lipid present in the nanoparticle.

In one embodiment, the amount of the cationic lipid is about 42.5 mol % of the total lipid present in the nanoparticle. In one embodiment, the amount of the cationic lipid is about 45 mol %. In one embodiment, the amount of the cationic lipid is about 47.5 mol %. In one embodiment, the amount of the cationic lipid is about 50 mol %. In one embodiment, the amount of the cationic lipid is about 52.5 mol %. In one embodiment, the amount of the cationic lipid is about 55 mol %. In one embodiment, the amount of the cationic lipid is about 57.5 mol %. In one embodiment, the amount of the cationic lipid is about 60 mol %. In one embodiment, the amount of the cationic lipid is about 62.5 mol %. In one embodiment, the amount of the cationic lipid is about 65 mol %. In one embodiment, the amount of the cationic lipid is about 67.5 mol %. In one embodiment, the amount of the cationic lipid is about 70 mol %.

In one embodiment, the amount of the polymer conjugated lipid is from about 0.5 mol % to about 2.5 mol % of the total lipid present in the nanoparticle. In one embodiment, the amount of the polymer conjugated lipid is from about 0.5 mol % to about 0.75 mol %, about 0.75 mol % to about 1 mol %, about 0.5 mol % to about 1 mol %, about 1 mol % to about 1.25 mol %, about 1.25 mol % to about 1.5 mol %, about 1 mol % to about 1.5 mol %, about 1.5 mol % to about 1.75 mol %, about 1.75 mol % to about 2 mol %, about 1 mol % to about 2 mol %, about 2 mol % to about 2.25 mol %, about 2.25 mol % to about 2.5 mol %, or about 2 mol % to about 2.5 mol % of the total lipid present in the nanoparticle. In one embodiment, the amount of the polymer conjugated lipid is from about 0.5 mol % to about 2 mol %. In one embodiment, the amount of the polymer conjugated lipid is from about 1 mol % to about 2.5 mol %. In one embodiment, the amount of the polymer conjugated lipid is from about 0.5 mol % to about 1.5 mol %. In one embodiment, the amount of the polymer conjugated lipid is from about 1 mol % to about 2 mol %. In one embodiment, the amount of the polymer conjugated lipid is from about 1.5 mol % to about 2.5 mol %. In one embodiment, the amount of the polymer conjugated lipid is from about 0.5 mol % to about 1 mol %. In one embodiment, the amount of the polymer conjugated lipid is from about 1 mol % to about 1.5 mol %. In one embodiment, the amount of the polymer conjugated lipid is from about 1.5 mol % to about 2 mol %. In one embodiment, the amount of the polymer conjugated lipid is from about 2 mol % to about 2.5 mol %.

In one embodiment, the amount of the polymer conjugated lipid is about 0.5 mol %, about 0.6 mol %, about 0.7 mol %, about 0.8 mol %, about 0.9 mol %, about 1 mol %, about 1.1 mol %, about 1.2 mol %, about 1.25 mol %, about 1.3 mol %, about 1.35 mol %, about 1.4 mol %, about 1.45 mol %, about 1.5 mol %, about 1.55 mol %, about 1.6 mol %, about 1.65 mol %, about 1.7 mol %, about 1.75 mol %, about 1.8 mol %, about 1.85 mol %, about 1.9 mol %, about 1.95 mol %, about 2 mol %, about 2.1 mol %, about 2.2 mol %, about 2.3 mol %, about 2.4 mol %, or about 2.5 mol % of the total lipid present in the nanoparticle.

In one embodiment, the amount of the polymer conjugated lipid is about 1 mol % of the total lipid present in the nanoparticle. In one embodiment, the amount of the polymer conjugated lipid is about 1.25 mol %. In one embodiment, the amount of the polymer conjugated lipid is about 1.5 mol %. In one embodiment, the amount of the polymer conjugated lipid is about 1.75 mol %. In one embodiment, the amount of the polymer conjugated lipid is about 2 mol %. In one embodiment, the amount of the polymer conjugated lipid is about 2.25 mol %. In one embodiment, the amount of the polymer conjugated lipid is about 2.5 mol %.

In one embodiment, the amount of the steroid is from about 22 mol % to about 59.5 mol % of total lipid present in the nanoparticle. In one embodiment, the amount of the steroid is from about 27 mol % to about 54.5 mol %. In one embodiment, the amount of the steroid is from about 32 mol % to about 49.5 mol %. In one embodiment, the amount of the steroid is from about 30 mol % to about 50 mol %. In one embodiment, the amount of the steroid is from about 33 mol % to about 44 mol %. In one embodiment, the amount of the steroid is from about 33 mol % to about 38 mol %. In one embodiment, the amount of the steroid is from about 38 mol % to about 48 mol %. In one embodiment, the amount of the steroid is from about 38 mol % to about 44 mol %. In one embodiment, the amount of the steroid is from about 44 mol % to about 49 mol %. In one embodiment, the amount of the steroid is the balance of total lipid present in the nanoparticle (other than the cationic lipid and the polymer conjugated lipid).

In one embodiment, the amount of the steroid is from about 22 mol % to about 23.5 mol %, about 23.5 mol % to about 29 mol %, about 29 mol % to about 33 mol %, about 33 mol % to about 36.5 mol %, about 36.5 mol % to about 38 mol %, about 38 mol % to about 42 mol %, about 42 mol % to about 44 mol %, about 33 mol % to about 44 mol %, about 44 mol % to about 49 mol %, about 49 mol % to about 54 mol % or about 54 mol % to about 59.5 mol % of the total lipid present in the nanoparticle.

In one embodiment, the amount of the steroid is about 33 mol % of the total lipid present in the nanoparticle. In one embodiment, the amount of the steroid is about 38 mol %. In one embodiment, the amount of the steroid is about 38.5 mol %. In one embodiment, the amount of the steroid is about 39 mol %. In one embodiment, the amount of the steroid is about 44 mol %. In one embodiment, the amount of the steroid is about 48 mol %. In one embodiment, the amount of the steroid is about 48.5 mol %. In one embodiment, the amount of the steroid is about 49 mol %.

In one embodiment, the amount of cationic lipid is from about 45 mol % to about 70 mol % of the total lipid present in the nanoparticle, and the amount of the polymer conjugated lipid is from about 1 mol % to about 2.5 mol % of the total lipid present in the nanoparticle.

In one embodiment, the amount of cationic lipid is from about 45 mol % to about 70 mol % of the total lipid present in the nanoparticle, and the amount of the polymer conjugated lipid is from about 1 mol % to about 2 mol % of the total lipid present in the nanoparticle.

In one embodiment, the amount of cationic lipid is from about 50 mol % to about 65 mol % of the total lipid present in the nanoparticle, and the amount of the polymer conjugated lipid is from about 1.5 mol % to about 2.5 mol % of the total lipid present in the nanoparticle.

In one embodiment, the amount of the cationic lipid is from about 45 mol % to about 65 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 45 mol % to about 65 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 50 mol % to about 65 mol % of the total lipid present in the nanoparticle, and the amount of the polymer conjugated lipid is from about 0.5 mol % to about 2.5 mol % of the total lipid present in the nanoparticle.

In one embodiment, the amount of the cationic lipid is from about 50 mol % to about 65 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 50 mol % to about 65 mol % of the total lipid present in the nanoparticle, and the amount of the polymer conjugated lipid is from about 1 mol % to about 1.5 mol % of the total lipid present in the nanoparticle.

In one embodiment, the amount of the cationic lipid is from about 50 mol % to about 65 mol % of the total lipid present in the nanoparticle, and the amount of the polymer conjugated lipid is from about 1.5 mol % to about 2 mol % of the total lipid present in the nanoparticle.

In one embodiment, the amount of the cationic lipid is from about 50 mol % to about 65 mol % of the total lipid present in the nanoparticle, and the amount of the polymer conjugated lipid is from about 2 mol % to about 2.5 mol % of the total lipid present in the nanoparticle.

In one embodiment, the amount of the cationic lipid is from about 50 mol % to about 60 mol % of the total lipid present in the nanoparticle, and the amount of the polymer conjugated lipid is from about 0.8 mol % to about 2 mol % of the total lipid present in the nanoparticle.

In one embodiment, the amount of the cationic lipid is from about 50 mol % to about 60 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 50 mol % to about 60 mol % of the total lipid present in the nanoparticle, and the amount of the polymer conjugated lipid is from about 1.5 mol % to about 2.5 mol % of the total lipid present in the nanoparticle.

In one embodiment, the amount of the cationic lipid is from about 50 mol % to about 60 mol % of the total lipid present in the nanoparticle, and the amount of the polymer conjugated lipid is from about 2 mol % to about 2.5 mol % of the total lipid present in the nanoparticle.

In one embodiment, the amount of the cationic lipid is from about 55 mol % to about 65 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 55 mol % to about 65 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 55 mol % to about 65 mol % of the total lipid present in the nanoparticle, and the amount of the polymer conjugated lipid is from about 0.5 mol % to about 2.5 mol % of the total lipid present in the nanoparticle.

In one embodiment, the amount of the cationic lipid is from about 55 mol % to about 65 mol % of the total lipid present in the nanoparticle, and the amount of the polymer conjugated lipid is from about 0.8 mol % to about 2 mol % of the total lipid present in the nanoparticle.

In one embodiment, the amount of the cationic lipid is from about 60 mol % to about 65 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 60 mol % to about 65 mol % of the total lipid present in the nanoparticle, and the amount of the polymer conjugated lipid is from about 1.5 mol % to about 2.5 mol % of the total lipid present in the nanoparticle.

In one embodiment, the amount of the cationic lipid is from about 45 mol % to about 60 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 45 mol % to about 60 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 45 mol % to about 55 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 45 mol % to about 50 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 50 mol % to about 55 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 50 mol % to about 55 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 50 mol % to about 55 mol % of the total lipid present in the nanoparticle, and the amount of the polymer conjugated lipid is from about 1.5 mol % to about 2.5 mol % of the total lipid present in the nanoparticle.

In one embodiment, the amount of the cationic lipid is from about 55 mol % to about 60 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 55 mol % to about 60 mol % of the total lipid present in the nanoparticle, and the amount of the polymer conjugated lipid is from about 1 mol % to about 2 mol % of the total lipid present in the nanoparticle.

In one embodiment, the amount of the cationic lipid is from about 55 mol % to about 60 mol % of the total lipid present in the nanoparticle, and the amount of the polymer conjugated lipid is from about 0.5 mol % to about 2.5 mol % of the total lipid present in the nanoparticle.

In one embodiment, the amount of the cationic lipid is from about 55 mol % to about 60 mol % of the total lipid present in the nanoparticle, and the amount of the polymer conjugated lipid is from about 0.8 mol % to about 2 mol % of the total lipid present in the nanoparticle.

In one embodiment, the amount of the cationic lipid is from about 60 mol % to about 70 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 40 mol % to about 55 mol % of the total lipid present in the nanoparticle, and the amount of the polymer conjugated lipid is from about 1 mol % to about 2.5 mol % of the total lipid present in the nanoparticle.

In one embodiment, the amount of the cationic lipid is from about 40 mol % to about 55 mol % of the total lipid present in the nanoparticle, and the amount of the polymer conjugated lipid is from about 1 mol % to about 2 mol % of the total lipid present in the nanoparticle.

In one embodiment, the amount of the cationic lipid is about 67.5 mol % of the total lipid present in the nanoparticle, and the amount of the polymer conjugated lipid is about 2 mol % of the total lipid present in the nanoparticle.

In one embodiment, the amount of the cationic lipid is about 65 mol % of the total lipid present in the nanoparticle, and the amount of the polymer conjugated lipid is about 1 mol % of the total lipid present in the nanoparticle.

In one embodiment, the amount of the cationic lipid is about 65 mol % of the total lipid present in the nanoparticle, and the amount of the polymer conjugated lipid is about 1.5 mol % of the total lipid present in the nanoparticle.

In one embodiment, the amount of the cationic lipid is about 65 mol % of the total lipid present in the nanoparticle, and the amount of the polymer conjugated lipid is about 2 mol % of the total lipid present in the nanoparticle.

In one embodiment, the amount of the cationic lipid is about 65 mol % of the total lipid present in the nanoparticle, and the amount of the polymer conjugated lipid is about 2.5 mol % of the total lipid present in the nanoparticle.

In one embodiment, the amount of the cationic lipid is about 60 mol % of the total lipid present in the nanoparticle, and the amount of the polymer conjugated lipid is about 1 mol % of the total lipid present in the nanoparticle.

In one embodiment, the amount of the cationic lipid is about 60 mol % of the total lipid present in the nanoparticle, and the amount of the polymer conjugated lipid is about 1.5 mol % of the total lipid present in the nanoparticle.

In one embodiment, the amount of the cationic lipid is about 60 mol % of the total lipid present in the nanoparticle, and the amount of the polymer conjugated lipid is about 2 mol % of the total lipid present in the nanoparticle.

In one embodiment, the amount of the cationic lipid is about 60 mol % of the total lipid present in the nanoparticle, and the amount of the polymer conjugated lipid is about 2.5 mol % of the total lipid present in the nanoparticle.

In one embodiment, the amount of the cationic lipid is about 55 mol % of the total lipid present in the nanoparticle, and the amount of the polymer conjugated lipid is about 1 mol % of the total lipid present in the nanoparticle.

In one embodiment, the amount of the cationic lipid is about 55 mol % of the total lipid present in the nanoparticle, and the amount of the polymer conjugated lipid is about 1.5 mol % of the total lipid present in the nanoparticle.

In one embodiment, the amount of the cationic lipid is about 55 mol % of the total lipid present in the nanoparticle, and the amount of the polymer conjugated lipid is about 2 mol % of the total lipid present in the nanoparticle.

In one embodiment, the amount of the cationic lipid is about 55 mol % of the total lipid present in the nanoparticle, and the amount of the polymer conjugated lipid is about 2.5 mol % of the total lipid present in the nanoparticle.

In one embodiment, the amount of the cationic lipid is about 50 mol % of the total lipid present in the nanoparticle, and the amount of the polymer conjugated lipid is about 1 mol % of the total lipid present in the nanoparticle.

In one embodiment, the amount of the cationic lipid is about 50 mol % of the total lipid present in the nanoparticle, and the amount of the polymer conjugated lipid is about 1.5 mol % of the total lipid present in the nanoparticle.

In one embodiment, the amount of the cationic lipid is about 50 mol % of the total lipid present in the nanoparticle, and the amount of the polymer conjugated lipid is about 2 mol % of the total lipid present in the nanoparticle.

In one embodiment, a lipid nanoparticle with the amount of the cationic lipid and the amount of the polymer conjugated lipid provided herein has an increased nanoparticle size as described herein or elsewhere (e.g., from about 100 nm to about 140 nm, or from about 110 nm to about 130 nm) or as illustrated in the Examples.

In one embodiment, the lipid component of the nanoparticle composition includes one or more polymer conjugated lipids, such as PEGylated lipids (PEG lipids). Without being bound by the theory, it is contemplated that a polymer conjugated lipid component in a nanoparticle composition can improve of colloidal stability, reduce protein absorption of the nanoparticles and/or increase blood circulation time of the nanoparticles.

In one embodiment, the polymer conjugated lipid is a pegylated lipid, for example, a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as o-methoxy(polyethoxy)ethyl-N-(2,3-di(tetradecanoxy)propyl)carbamate or 2,3-di(tetradecanoxy)propyl-N-(ω-methoxy(polyethoxy)ethyl)carbamate.

In one embodiment, the polymer conjugated lipid is a polyethylene glycol (PEG)-conjugated lipid. In one embodiment, the polymer conjugated lipid is a PEG-diacylglycerol (PEG-DAG) conjugate, a PEG-dialkyloxypropyl (PEG-DAA) conjugate, a PEG-dimyristyloxypropyl (PEG-DMA) conjugate, a PEG-distearyloxypropyl (PEG-DSA) conjugate, or a mixture thereof. In one embodiment, the polymer conjugated lipid is PEG-DAG. In one embodiment, the polymer conjugated lipid is PEG-DAA. In one embodiment, the polymer conjugated lipid is PEG-DMA. In one embodiment, the polymer conjugated lipid is PEG-DSA. In one embodiment, the polymer conjugated lipid is PEG-DMG. In one embodiment, the polymer conjugated lipid is PEG-DMPE. In one embodiment, the polymer conjugated lipid is DMG-PEG2000. In one embodiment, the polymer conjugated lipid is DMPE-PEG2000.

In one embodiment, the polymer conjugated lipid is a polyethylene glycol (PEG)-conjugated lipid, wherein the molecular weight of PEG is about 500 g/mol, about 750 g/mol, about 1000 g/mol, about 1500 g/mol, about 2000 g/mol, about 2500 g/mol, about 3000 g/mol, about 3500 g/mol, about 4000 g/mol, about 4500 g/mol, about 5000 g/mol, or about 10000 g/mol.

In one embodiment, the polymer conjugated lipid is selected from the group consisting of DMG-PEG500, DMG-PEG1000, DMG-PEG1500, DMG-PEG2000, DMG-PEG2500, DMG-PEG3000, DMG-PEG5000 and DMG-PEG10000. In one embodiment, the polymer conjugated lipid is selected from the group consisting of DMPE-PEG500, DMPE-PEG1000, DMPE-PEG1500, DMPE-PEG2000, DMPE-PEG2500, DMPE-PEG3000, DMPE-PEG5000 and DMPE-PEG10000.

In one embodiment, the steroid is glucocorticoid, mineralocorticoid, clobetasol, cholesterol or a cholesterol derivative. In one embodiment, the steroid is cholesterol. In one embodiment, the steroid is cholesterol derivative. In one embodiment, the steroid is glucocorticoid. In one embodiment, the steroid is mineralocorticoid. In one embodiment, the steroid is clobetasol. In one embodiment, the steroid is a mixture of glucocorticoid, mineralocorticoid, clobetasol, cholesterol or a cholesterol derivative.

In one embodiment, the polymer conjugated lipid is a PEG-conjugated lipid, and the steroid is cholesterol or a cholesterol derivative. In one embodiment, the polymer conjugated lipid is DMG-PEG2000, and the steroid is cholesterol or a cholesterol derivative. In one embodiment, the polymer conjugated lipid is DMPE-PEG2000, and the steroid is cholesterol or a cholesterol derivative.

In one embodiment, the cationic lipid has a pKa of at least 6. In one embodiment, the cationic lipid has a pKa of at least 7. In one embodiment, the cationic lipid has a pKa of at least 8. In one embodiment, the cationic lipid has a pKa of at least 9. In one embodiment, the cationic lipid has a pKa of at least 9.5. In one embodiment, the cationic lipid has a pKa of at least 10. In one embodiment, the cationic lipid has a pKa of at least 11. In one embodiment, the cationic lipid has a pKa of at least 12.

In one embodiment, the cationic lipid comprises only one ester moiety that is the C₁₄-C₄₂ alkyl ester moiety. In one embodiment, the ester moiety is a C₁₄-C₃₇ alkyl ester moiety. In one embodiment, the ester moiety is a C₁₄-C₃₂ alkyl ester moiety. In one embodiment, the ester moiety is a C₁₄-C₂₇ alkyl ester moiety. In one embodiment, the ester moiety is a C₁₄-C₂₂ alkyl ester moiety. In one embodiment, the ester moiety is a C₁₄-C₁₇ alkyl ester moiety. In one embodiment, the ester moiety is a C₁₅ alkyl ester moiety. In one embodiment, the ester moiety is a C₁₆ alkyl ester moiety. In one embodiment, the ester moiety is a C₁₇ alkyl ester moiety.

In one embodiment, the cationic lipid comprises the C₁₄-C₄₂ alkyl ester moiety and at least a second ester moiety. In one embodiment, the cationic lipid comprises the C₁₄-C₄₂ alkyl ester moiety and a second alkyl ester moiety. In one embodiment, the cationic lipid comprises two C₁₄-C₄₂ alkyl ester moieties. In one embodiment, the two C₁₄-C₄₂ alkyl ester moieties are identical. In one embodiment, the two C₁₄-C₄₂ alkyl ester moieties are different. In one embodiment, the cationic lipid comprises two C₁₅ alkyl ester moieties. In one embodiment, the cationic lipid comprises two C₁₆ alkyl ester moieties. In one embodiment, the cationic lipid comprises a C₁₀ alkyl ester moiety and a C₁₇ alkyl ester moiety.

In one embodiment, at least one ester moiety of the cationic lipid is —C(═O)OR¹ or —OC(═O)R¹, wherein R¹ is a branched C₁₄-C₄₂ alkyl. In one embodiment, R¹ is a branched C₁₄-C₁₉ alkyl. In one embodiment, R¹ is a branched C₂₀-C₂₅ alkyl. In one embodiment, R¹ is a branched C₂₆-C₃₁ alkyl. In one embodiment, R¹ is a branched C₃₂-C₃₇ alkyl. In one embodiment, R¹ is a branched C₃₈-C₄₂ alkyl. In one embodiment, R¹ is a branched C₁₅ alkyl ester moiety. In one embodiment, R¹ is a branched C₁₆ alkyl ester moiety. In one embodiment, R¹ is a branched C₁₇ alkyl ester moiety.

In one embodiment, the second ester moiety of the cationic lipid is —C(═O)OR² or —OC(═O)R², wherein R² is a straight or branched C₆-C₄₂ alkyl. In one embodiment, R² is a straight or branched C₆-C₁₁ alkyl. In one embodiment, R² is a straight or branched C₁₂-C₁₇ alkyl. In one embodiment, R² is a straight or branched C₁₈-C₂₃ alkyl. In one embodiment, R² is a straight or branched C₂₄-C₂₉ alkyl. In one embodiment, R² is a straight or branched C₃₀-C₃₅ alkyl. In one embodiment, R² is a straight or branched C₃₆-C₄₂ alkyl.

In one embodiment, the second ester moiety of the cationic lipid is —C(═O)OR² or —OC(═O)R², wherein R² is a straight C₆-C₄₂ alkyl. In one embodiment, R² is a straight C₆-C₁₂ alkyl. In one embodiment, R² is a straight C₁₃-C₁₈ alkyl. In one embodiment, R² is a straight C₁₉-C₂₅ alkyl. In one embodiment, R² is a straight C₂₆-C₃₂ alkyl. In one embodiment, R² is a straight C₃₂-C₄₂ alkyl. In one embodiment, R² is a straight C₉ alkyl. In one embodiment, R² is a straight C₁₀ alkyl. In one embodiment, R² is a straight C₁₁ alkyl.

In one embodiment, the second ester moiety of the cationic lipid is —C(═O)OR² or —OC(═O)R², wherein R² is a branched C₆-C₁₂ alkyl. In one embodiment, R² is a branched C₁₃-C₂₄ alkyl. In one embodiment, R² is a branched C₂₅-C₄₂ alkyl. In one embodiment, R² is a branched C₁₅ alkyl. In one embodiment, R² is a branched C₁₆ alkyl. In one embodiment, R² is a branched C₁₇ alkyl.

In one embodiment, the cationic lipid comprises the C₁₄-C₄₂ alkyl ester moiety and at least a second ester moiety and a third ester moiety. In one embodiment, the cationic lipid comprises the C₁₄-C₄₂ alkyl ester moiety, a second alkyl ester moiety and a third ester moiety. In one embodiment, the second alkyl ester moiety and the third alkyl ester moiety are independently —C(═O)OR² or —OC(═O)R², wherein each R² is independently a straight C₇-C₁₁ alkyl. In one embodiment, the second alkyl ester moiety and the third alkyl ester moiety are part of a

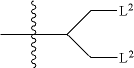

moiety, wherein each L² is independently —OC(═O)R², and each R² is independently straight C₇-C₁₁ alkyl. In one embodiment, the second alkyl ester moiety and the third alkyl ester moiety are part of a

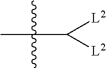

moiety, wherein each L² is independently —C(═O)OR², and each R² is independently straight C₇-C₁₁ alkyl.

In one embodiment, the cationic lipid comprises only one tertiary amine. In one embodiment, the cationic lipid comprises two tertiary amines. In one embodiment, the tertiary amine is protonated. In one embodiment, the tertiary amine is positively charged. In one embodiment, the tertiary amine exists in the form of quaternary ammonium salt. In one embodiment, the tertiary amine is protonated when the pH is from about 5 to about 9. In one embodiment, the tertiary amine is protonated at pH 5, pH 5.5, pH 6, pH 6.5, pH 7, pH 7.5, pH 8, pH 8.5 or pH 9.

In one embodiment, the cationic lipid comprises at least one tertiary amine and at least one C₁₄-C₄₂ alkyl ester moiety. In one embodiment, the cationic lipid comprises one tertiary amine and two C₁₄-C₄₂ alkyl ester moieties. In one embodiment, the cationic lipid comprises two tertiary amines and two C₁₄-C₄₂ alkyl ester moieties. In one embodiment, the cationic lipid comprises one tertiary amine, one branched C₁₄-C₄₂ alkyl ester moiety and one straight C₆-C₄₂ alkyl ester moiety. In one embodiment, the cationic lipid comprises two tertiary amines, one C₁₄-C₄₂ alkyl ester moiety, a straight C₇-C₁₁ alkyl second ester moiety and a straight C₇-C₁₁ alkyl third ester moiety.

In one embodiment, the cationic lipid comprises a moiety of

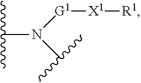

wherein G¹ is C₂-C₁₂ alkylene; X¹ is —C(═O)O— or —OC(═O)—; and R¹ is a branched C₁₄-C₄₂ alkyl. In one embodiment, G¹ is C₂-C₅ alkylene. In one embodiment, G¹ is C₆-C₉ alkylene. In one embodiment, G¹ is C₁₀-C₁₂ alkylene.

In one embodiment, G¹ is C₂-C₁₂ alkylene, X¹ is —C(═O)O— or —OC(═O)—; and R¹ is a branched C₁₄-C₁₉ alkyl. In one embodiment, R¹ is a branched C₂₀-C₂₅ alkyl. In one embodiment, R¹ is a branched C₂₆-C₃₁ alkyl. In one embodiment, R¹ is a branched C₃₂-C₃₇ alkyl. In one embodiment, R¹ is a branched C₃₈-C₄₂ alkyl.

In one embodiment, the cationic lipid is of Formula (I):

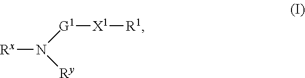

or a pharmaceutically acceptable salt, prodrug or stereoisomer thereof, wherein:
G¹ is C₂-C₁₂ alkylene;
X¹ is —C(═O)O— or —OC(═O)—;
R¹ is a branched C₁₄-C₄₂ alkyl;
R^{y} is C₁-C₆ alkyl or -G²-X²—R².
G² is C₂-C₁₂ alkylene;
X² is —C(═O)O— or —OC(═O)—;
R² is a straight or branched C₆-C₄₂ alkyl;
R^{x} is C₁-C₆ alkyl or -G³-R³;
G³ is C₂-C₁₂ alkylene;
R³ is —N(R⁴)R⁵ or —OR⁶;
R⁴ is C₁-C₁₂ alkyl, C₃-C₈ cycloalkyl, C₃-C₈ cycloalkenyl, or C₆-C₁₀ aryl;
R⁵ is C₁-C₁₂ alkyl, C₃-C₈ cycloalkyl, C₃-C₈ cycloalkenyl, or C₆-C₁₀ aryl;
or R⁴ and R⁵ together with the nitrogen to which they are attached form a cyclic moiety;
R⁶ is hydrogen, C₁-C₁₂ alkyl, C₃-C₈ cycloalkyl, C₃-C₈ cycloalkenyl, or C₆-C₁₀ aryl; and
wherein each alkyl, alkenyl, cycloalkyl, cycloalkenyl, aryl, alkylene, and cyclic moiety is independently optionally substituted.

In one embodiment, the cationic lipid is of Formula (II):

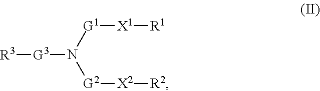

or a pharmaceutically acceptable salt, prodrug or stereoisomer thereof.

In one embodiment, the cationic lipid is of Formula (II-A):

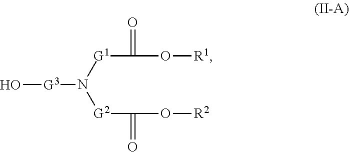

or a pharmaceutically acceptable salt, prodrug or stereoisomer thereof, wherein:
G¹ is C₅-C₇ alkylene;
R¹ is a branched C₁₄-C₁₉ alkyl;
G² is C₅-C₇ alkylene;
R² is a straight C₇-C₁₃ alkyl;
G³ is C₂-C₄ alkylene.

In one embodiment, the cationic lipid is of Formula (II-A-1):

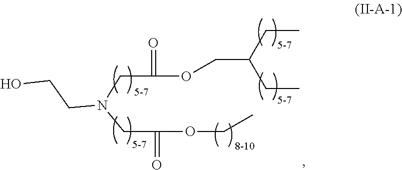

or a pharmaceutically acceptable salt, pro rug or stereoisomer thereof.

In one embodiment, the cationic lipid is of Formula (II-B):

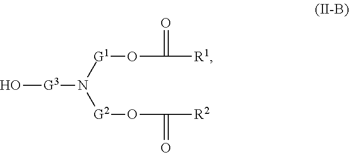

or a pharmaceutically acceptable salt, prodrug or stereoisomer thereof, wherein:
G¹ is C₅-C₇ alkylene;
R¹ is a branched C₁₄-C₁₉ alkyl;
G² is C₅-C₇ alkylene;
R² is a branched C₁₄-C₁₉ alkyl;
G³ is C₂-C₄ alkylene.

In one embodiment, the cationic lipid is of Formula (II-B-1):

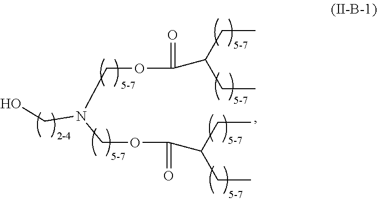

or a pharmaceutically acceptable salt, prodrug or stereoisomer thereof.

In one embodiment, the cationic lipid is of Formula (II-C):

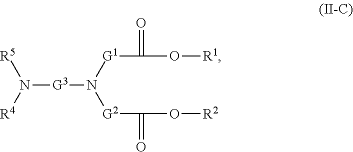

or a pharmaceutically acceptable salt, prodrug or stereoisomer thereof, wherein:
G¹ is C₅-C₇ alkylene;
R¹ is a branched C₁₄-C₁₉ alkyl;
G² is C₅-C₇ alkylene;
R² is a branched C₁₄-C₁₉ alkyl;
G³ is C₂-C₄ alkylene;
R⁴ is C₃-C₈ cycloalkyl; and
R⁵ is C₂-C₄ alkyl substituted with a hydroxy.

In one embodiment, the cationic lipid is of Formula (II-C-1):

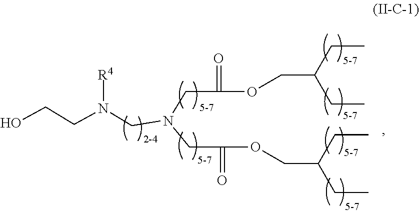

or a pharmaceutically acceptable salt, prodrug or stereoisomer thereof.

In one embodiment, the cationic lipid is of Formula (III):

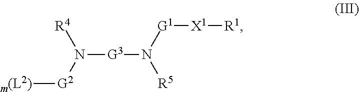

or a pharmaceutically acceptable salt, prodrug or stereoisomer thereof, wherein:
G¹ is C₂-C₁₂ alkylene;
Xⁱ is —C(═O)O— or —OC(═O)—;
R¹ is a branched C₁₄-C₄₂ alkyl;
G² is C₂-C₁₂ alkylene;
each L² is independently —X²—R²;
each X² is independently —C(═O)O— or —OC(═O)—;
each R² is independently a straight or branched C₆-C₄₂ alkyl;
m is 1 or 2;
G³ is C₂-C₁₂ alkylene;
R⁴ is C₁-C₁₂ alkyl, C₃-C₈ cycloalkyl, C₃-C₈ cycloalkenyl, or C₆-C₁₀ aryl;
R⁵ is C₁-C₁₂ alkyl, C₃-C₈ cycloalkyl, C₃-C₈ cycloalkenyl, or C₆-C₁₀ aryl; and
wherein each alkyl, alkenyl, cycloalkyl, cycloalkenyl, aryl, alkylene, and cyclic moiety is independently optionally substituted.

In one embodiment, m is 1. In one embodiment, m is 2.

In one embodiment, the cationic lipid is of Formula (IV):

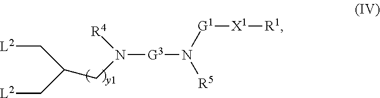

or a pharmaceutically acceptable salt, prodrug or stereoisomer thereof, wherein:
y1 is an integer from 0 to 9.

In one embodiment, y1 is an integer from 2 to 6. In one embodiment, y1 is 2. In one embodiment, y1 is 3. In one embodiment, y1 is 4. In one embodiment, y1 is 5. In one embodiment, y1 is 6.

In one embodiment, each L² is independently —OC(═O)R². In one embodiment, each L² is independently —OC(═O)R², and each R² is independently straight C₇-C₁₁ alkyl. In one embodiment, each L² is independently —C(═O)OR². In one embodiment, each L² is independently —C(═O)OR², and each R² is independently straight C₇-C₁₁ alkyl.

In one embodiment, the cationic lipid is of Formula (IV-A):

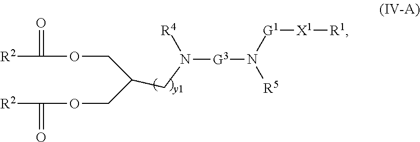

or a pharmaceutically acceptable salt, prodrug or stereoisomer thereof, wherein:
G¹ is C₅-C₇ alkylene;
R¹ is a branched C₁₄-C₁₉ alkyl;
each R² is independently a straight C₇-C₁₃ alkyl;
G³ is C₂-C₄ alkylene;
R⁴ is C₃-C₈ cycloalkyl;
R⁵ is C₂-C₄ alkyl substituted with a hydroxy; and
y1 is an integer from 2 to 6.

In one embodiment, the cationic lipid is of Formula (IV-A-1):

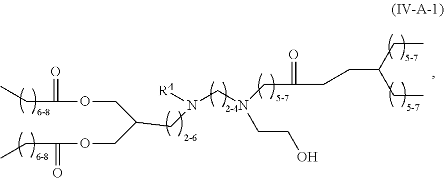

or a pharmaceutically acceptable salt, prodrug or stereoisomer thereof.

In one embodiment, G¹ is C₂-C₅ alkylene. In one embodiment, G¹ is C₅-C₇ alkylene. In one embodiment, G¹ is C₆-C₉ alkylene. In one embodiment, G¹ is C₁₀-C₁₂ alkylene. In one embodiment, G¹ is C₂ alkylene. In one embodiment, G¹ is C₃ alkylene. In one embodiment, G¹ is C₄ alkylene. In one embodiment, G¹ is C₅ alkylene. In one embodiment, G¹ is C₆ alkylene. In one embodiment, G¹ is C₇ alkylene. In one embodiment, G¹ is Cₛ alkylene. In one embodiment, G¹ is C₉ alkylene. In one embodiment, G¹ is C₁₀ alkylene. In one embodiment, G¹ is C₁₁ alkylene. In one embodiment, G¹ is C₁₂ alkylene.

In one embodiment, X¹ is —C(═O)O—. In one embodiment, X¹ is —OC(═O)—. Unless otherwise specified, the left side is toward G¹, and the right side is toward R¹.

In one embodiment, R¹ is a branched C₁₄-C₂₄ alkyl. In one embodiment, R¹ is a branched C₁₄-C₁₉ alkyl. In one embodiment, R¹ is a branched C₂₀-C₂₅ alkyl. In one embodiment, R¹ is a branched C₂₆-C₃₁ alkyl. In one embodiment, R¹ is a branched C₃₂-C₃₇ alkyl. In one embodiment, R¹ is a branched C₃₈-C₄₂ alkyl. In one embodiment, R¹ is —R⁷—CH(R⁸)(R⁹), wherein R⁷ is C₀-C₅ alkylene, and R⁸ and R⁹ are independently C₂-C₁₀ alkyl. In one embodiment, R¹ is —R⁷—CH(R⁸)(R⁹), wherein R⁷ is C₀-C₁ alkylene, and R⁸ and R⁹ are independently C₄-C₈ alkyl.

In one embodiment, R¹ is a branched C₁₄ alkyl. In one embodiment, R¹ is a branched C₁₅ alkyl. In one embodiment, R¹ is a branched C₁₆ alkyl. In one embodiment, R¹ is a branched C₁₇ alkyl. In one embodiment, R¹ is a branched Cis alkyl. In one embodiment, R¹ is a branched C₁₉ alkyl. In one embodiment, R¹ is a branched C₂₀ alkyl. In one embodiment, R¹ is a branched C₂₂ alkyl. In one embodiment, R¹ is a branched C₂₄ alkyl. In one embodiment, R¹ is a branched C₂₆ alkyl. In one embodiment, R¹ is a branched C₂₈ alkyl. In one embodiment, R¹ is a branched C₃₀ alkyl. In one embodiment, R¹ is a branched C₃₂ alkyl. In one embodiment, R¹ is a branched C₃₄ alkyl. In one embodiment, R¹ is a branched C₃₆ alkyl. In one embodiment, R¹ is a branched C₃₈ alkyl. In one embodiment, R¹ is a branched C₄₀ alkyl. In one embodiment, R¹ is a branched C₄₂ alkyl.

In one embodiment, R^{y} is C₁-C₆ alkyl. In one embodiment, R^{y} is methyl. In one embodiment, R^{y} is ethyl. In one embodiment, R^{y} is C₃ alkyl. In one embodiment, R^{y} is C₄ alkyl. In one embodiment, R^{y} is C₅ alkyl. In one embodiment, R^{y} is C₆ alkyl.

In one embodiment, R^{y} is -G²-X²—R².

In one embodiment, G² is C₂-C₁₂ alkylene. In one embodiment, G² is C₂-C₅ alkylene. In one embodiment, G² is C₅-C₇ alkylene. In one embodiment, G² is C₆-C₉ alkylene. In one embodiment, G² is C₁₀-C₁₂ alkylene. In one embodiment, G² is C₂ alkylene. In one embodiment, G² is C₃ alkylene. In one embodiment, G² is C₄ alkylene. In one embodiment, G² is C₅ alkylene. In one embodiment, G² is C₆ alkylene. In one embodiment, G² is C₇ alkylene. In one embodiment, G² is Cₛ alkylene. In one embodiment, G² is C₉ alkylene. In one embodiment, G² is C₁₀ alkylene. In one embodiment, G² is C₁₁ alkylene. In one embodiment, G² is C₁₂ alkylene.

In one embodiment, G² is

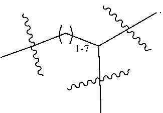

In one embodiment, G² is

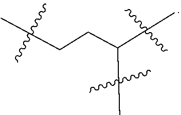 [SMILES: *CCC(*)*]

In one embodiment, G² is

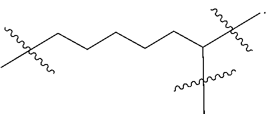 [SMILES: *CCCCCC(*)*]

In one embodiment, G² is

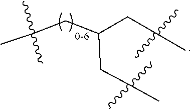

In one embodiment, G² is

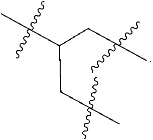 [SMILES: *CC(*)C*]

In one embodiment, G² is

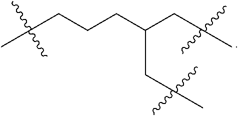 [SMILES: *CCCC(C*)C*]

In one embodiment, G² is

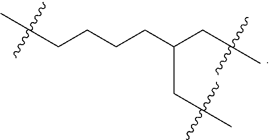 [SMILES: *CCCCC(C*)C*]

In one embodiment, X² is —C(═O)O—. In one embodiment, X² is —OC(═O)—. Unless otherwise specified, the left side is toward G², and the right side is toward R².

In one embodiment, R² is a straight or branched C₆-C₁₁ alkyl. In one embodiment, R² is a straight or branched C₁₂-C₁₇ alkyl. In one embodiment, R² is a straight or branched C₁₈-C₂₃ alkyl. In one embodiment, R² is a straight or branched C₂₄-C₂₉ alkyl. In one embodiment, R² is a straight or branched C₃₀-C₃₅ alkyl. In one embodiment, R² is a straight or branched C₃₆-C₄₂ alkyl.

In one embodiment, R² is a straight C₆-C₂₄ alkyl. In one embodiment, R² is straight C₇-C₁₃ alkyl. In one embodiment, R² is straight C₇-C₁₁ alkyl. In one embodiment, R² is a straight C₈ alkyl. In one embodiment, R² is a straight C₉ alkyl. In one embodiment, R² is a straight C₁₀ alkyl. In one embodiment, R² is a straight C₁₁ alkyl. In one embodiment, R² is a straight C₁₂ alkyl. In one embodiment, R² is a straight C₁₃ alkyl. In one embodiment, R² is a straight C₁₄ alkyl. In one embodiment, R² is a straight C₁₅ alkyl. In one embodiment, R² is a straight C₁₆ alkyl.

In one embodiment, R² is a branched C₁₄-C₂₄ alkyl. In one embodiment, R² is a branched C₁₄-C₁₉ alkyl. In one embodiment, R² is —R⁷—CH(R⁸)(R⁹), wherein R⁷ is C₀-C₅ alkylene, and R⁸ and R⁹ are independently C₂-C₁₀ alkyl. In one embodiment, R² is —R⁷—CH(R⁸)(R⁹), wherein R⁷ is C₀-C₁ alkylene, and R⁸ and R⁹ are independently C₄-C₈ alkyl.

In one embodiment, R² is a branched C₆ alkyl. In one embodiment, R² is a branched C₈ alkyl. In one embodiment, R² is a branched C₁₀ alkyl. In one embodiment, R² is a branched C₁₂ alkyl. In one embodiment, R² is a branched C₁₄ alkyl. In one embodiment, R² is a branched C₁₅ alkyl. In one embodiment, R² is a branched C₁₆ alkyl. In one embodiment, R² is a branched C₁₇ alkyl. In one embodiment, R² is a branched C₁₈ alkyl. In one embodiment, R² is a branched C₁₉ alkyl. In one embodiment, R² is a branched C₂₀ alkyl. In one embodiment, R² is a branched C₂₂ alkyl. In one embodiment, R² is a branched C₂₄ alkyl. In one embodiment, R² is a branched C₂₆ alkyl. In one embodiment, R² is a branched C₂₈ alkyl. In one embodiment, R² is a branched C₃₀ alkyl. In one embodiment, R² is a branched C₃₂ alkyl. In one embodiment, R² is a branched C₃₄ alkyl. In one embodiment, R² is a branched C₃₆ alkyl. In one embodiment, R² is a branched C₃₈ alkyl. In one embodiment, R² is a branched C₄₀ alkyl. In one embodiment, R² is a branched C₄₂ alkyl.

In one embodiment, R¹, R² are each independently one of the following structures:

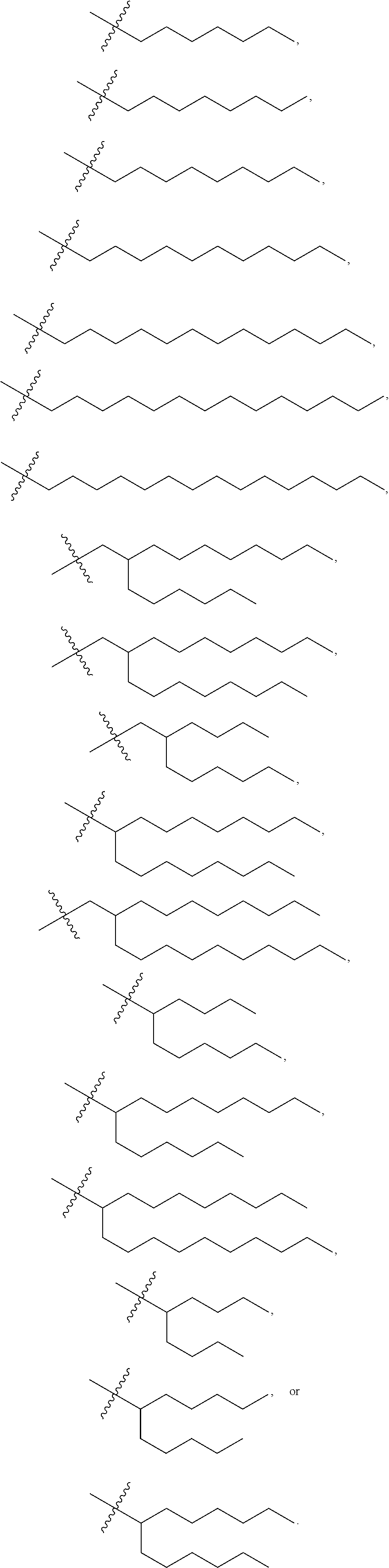

In one embodiment, R^{X} is C₁-C₆ alkyl. In one embodiment, R^{x} is methyl. In one embodiment, R^{x} is ethyl. In one embodiment, R^{x} is C₃ alkyl. In one embodiment, R^{x} is C₄ alkyl. In one embodiment, R^{x} is Cₛ alkyl. In one embodiment, R^{x} is C₆ alkyl.

In one embodiment, R^{x} is -G³-R³.

In one embodiment, G³ is C₂-C₁₂ alkylene. In one embodiment, G³ is C₂-C₄ alkylene. In one embodiment, G³ is C₂-C₅ alkylene. In one embodiment, G³ is C₆-C₉ alkylene. In one embodiment, G³ is C₁₀-C₁₂ alkylene. In one embodiment, G³ is C₂ alkylene. In one embodiment, G³ is C₃ alkylene. In one embodiment, G³ is C₄ alkylene. In one embodiment, G³ is C₅ alkylene. In one embodiment, G³ is C₆ alkylene. In one embodiment, G³ is C₇ alkylene. In one embodiment, G³ is Cₛ alkylene. In one embodiment, G³ is C₉ alkylene. In one embodiment, G³ is C₁₀ alkylene. In one embodiment, G³ is C₁₁ alkylene. In one embodiment, G³ is C₁₂ alkylene.

In one embodiment, R³ is —N(R⁴)R⁵.

In one embodiment, R⁴ is C₁-C₁₂ alkyl, C₃-C₈ cycloalkyl, C₃-C₈ cycloalkenyl, or C₆-C₁₀ aryl.

In one embodiment, R⁴ is C₁-C₁₂ alkyl. In one embodiment, R⁴ is C₁-C₈ alkyl. In one embodiment, R⁴ is C₁-C₆ alkyl. In one embodiment, R⁴ is C₁-C₄ alkyl. In one embodiment, R⁴ is methyl. In one embodiment, R⁴ is ethyl. In one embodiment, R⁴ is n-propyl. In one embodiment, R⁴ is iso-propyl. In one embodiment, R⁴ is n-butyl. In one embodiment, R⁴ is n-pentyl. In one embodiment, R⁴ is n-hexyl. In one embodiment, R⁴ is n-octyl. In one embodiment, R⁴ is n-nonyl.

In one embodiment, R⁴ is C₃-C₈ cycloalkyl. In one embodiment, R⁴ is cyclopropyl. In one embodiment, R⁴ is cyclobutyl. In one embodiment, R⁴ is cyclopentyl. In one embodiment, R⁴ is cyclohexyl. In one embodiment, R⁴ is cycloheptyl. In one embodiment, R⁴ is cyclooctyl.

In one embodiment, R⁴ is C₃-C₈ cycloalkenyl. In one embodiment, R⁴ is cyclopropenyl. In one embodiment, R⁴ is cyclobutenyl. In one embodiment, R⁴ is cyclopentenyl. In one embodiment, R⁴ is cyclohexenyl. In one embodiment, R⁴ is cycloheptenyl. In one embodiment, R⁴ is cyclooctenyl.

In one embodiment, R⁴ is C₆-C₁₀ aryl. In one embodiment, R⁴ is phenyl.

In one embodiment, R⁴ is unsubstituted.

In one embodiment, R⁴ is substituted with one or more substituents selected from the group consisting of oxo, —OR^{g}, —NR^{g}C(═O)R^{h}, —C(═O)NR^{g}R^{h}, —C(═O)R^{h}, —OC(═O)R^{h}, —C(═O)OR^{h} and —O—Rⁱ—OH, wherein:
R^{g} is at each occurrence independently H or C₁-C₆ alkyl;
R^{h} is at each occurrence independently C₁-C₆ alkyl; and
Rⁱ is at each occurrence independently C₁-C₆ alkylene.

In one embodiment, R⁴ is substituted with one or more hydroxyl. In one embodiment, R⁴ is substituted with one hydroxyl. In one embodiment, R⁴ is substituted with one or more hydroxyl and one or more oxo. In one embodiment, R⁴ is substituted with one hydroxyl and one oxo.

In one embodiment, R^{s} is C₁-C₁₂ alkyl, C₃-C₈ cycloalkyl, C₃-C₈ cycloalkenyl, or C₆-C₁₀ aryl.

In one embodiment, R⁵ is C₁-C₁₂ alkyl. In one embodiment, R⁵ is C₁-C₈ alkyl. In one embodiment, R⁵ is C₁-C₆ alkyl. In one embodiment, R⁵ is C₁-C₄ alkyl. In one embodiment, R⁵ is C₂-C₄ alkyl. In one embodiment, R⁵ is methyl. In one embodiment, R⁵ is ethyl. In one embodiment, R⁵ is n-propyl. In one embodiment, R⁵ is iso-propyl. In one embodiment, R⁵ is n-butyl. In one embodiment, R⁵ is n-pentyl. In one embodiment, R⁵ is n-hexyl. In one embodiment, R⁵ is n-octyl. In one embodiment, R⁵ is n-nonyl.

In one embodiment, R⁵ is C₃-C₈ cycloalkyl. In one embodiment, R⁵ is cyclopropyl. In one embodiment, R⁵ is cyclobutyl. In one embodiment, R⁵ is cyclopentyl. In one embodiment, R⁵ is cyclohexyl. In one embodiment, R⁵ is cycloheptyl. In one embodiment, R⁵ is cyclooctyl.

In one embodiment, R⁵ is C₃-C₈ cycloalkenyl. In one embodiment, R⁵ is cyclopropenyl. In one embodiment, R⁵ is cyclobutenyl. In one embodiment, R⁵ is cyclopentenyl. In one embodiment, R⁵ is cyclohexenyl. In one embodiment, R⁵ is cycloheptenyl. In one embodiment, R⁴ is cyclooctenyl.

In one embodiment, R⁵ is C₆-C₁₀ aryl. In one embodiment, R⁵ is phenyl.

In one embodiment, R⁴ and R⁵ together with the nitrogen to which they are attached form a cyclic moiety.

In one embodiment, the cyclic moiety (formed by R⁴ and R⁵ together with the nitrogen to which they are attached) is heterocyclyl. In one embodiment, the cyclic moiety is heterocycloalkyl. In one embodiment, the cyclic moiety is 4- to 8-membered heterocycloalkyl. In one embodiment, the cyclic moiety is 4-membered heterocycloalkyl. In one embodiment, the cyclic moiety is 5-membered heterocycloalkyl. In one embodiment, the cyclic moiety is 6-membered heterocycloalkyl. In one embodiment, the cyclic moiety is 7-membered heterocycloalkyl. In one embodiment, the cyclic moiety is 8-membered heterocycloalkyl.

In one embodiment, the cyclic moiety (formed by R⁴ and R⁵ together with the nitrogen to which they are attached) is azetidin-1-yl. In one embodiment, the cyclic moiety is pyrrolidin-1-yl. In one embodiment, the cyclic moiety is piperidin-1-yl. In one embodiment, the cyclic moiety is azepan-1-yl. In one embodiment, the cyclic moiety is azocan-1-yl. In one embodiment, the cyclic moiety is morpholinyl. In one embodiment, the cyclic moiety is piperazin-1-yl. The point of attachment in these groups is to G³.

As described herein and unless otherwise specified, the substitution patterns for R⁵ also applies to the cyclic moiety formed by R⁴ and R⁵ together with the nitrogen to which they are attached.

In one embodiment, R⁵ is unsubstituted.

In one embodiment, R⁵ is substituted with one or more substituents selected from the group consisting of oxo, —OR⁹, —NR^{g}C(═O)R^{h}, —C(═O)NR^{g}R^{h}, —C(═O)R^{h}, —OC(═O)R^{h}, —C(═O)OR^{h} and —O—Rⁱ—OH, wherein:
R^{g} is at each occurrence independently H or C₁-C₆ alkyl;
R^{h} is at each occurrence independently C₁-C₆ alkyl; and
Rⁱ is at each occurrence independently C₁-C₆ alkylene.

In one embodiment, R⁵ is substituted with one or more hydroxyl. In one embodiment, R⁵ is substituted with one hydroxyl.

In one embodiment, R⁵ is substituted with one or more hydroxyl and one or more oxo. In one embodiment, R⁵ is substituted with one hydroxyl and one oxo. In one embodiment, R⁵ is C₂-C₄ alkyl substituted with a hydroxy. In one embodiment, R⁵ is —CH₂CH₂OH.

In one embodiment, R³ is —OR⁶.

In one embodiment, R⁶ is hydrogen (i.e., R³ is —OH). In one embodiment, R⁶ is C₁-C₁₂ alkyl. In one embodiment, R⁶ is C₁-C₈ alkyl. In one embodiment, R⁶ is C₁-C₆ alkyl. In one embodiment, R⁶ is C₁-C₄ alkyl. In one embodiment, R⁶ is methyl. In one embodiment, R⁶ is ethyl. In one embodiment, R⁶ is C₃-C₈ cycloalkyl. In one embodiment, R⁶ is C₃-C₈ cycloalkenyl. In one embodiment, R⁶ is C₆-C₁₀ aryl. In one embodiment, R⁶ is phenyl. In one embodiment, R⁶ is unsubstituted. In one embodiment, R⁶ is substituted.

In one embodiment, the cationic lipid is

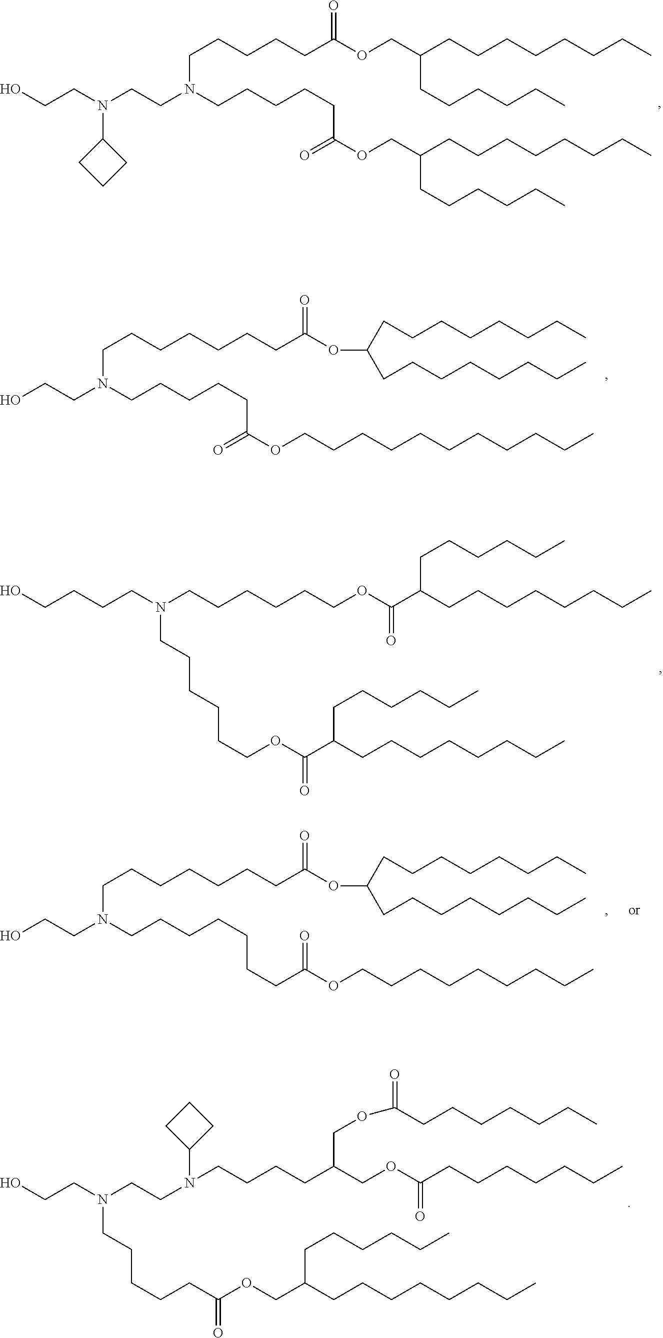

It is understood that any embodiment of the compounds provided herein, as set forth above, and any specific substituent and/or variable in the compound provided herein, as set forth above, may be independently combined with other embodiments and/or substituents and/or variables of the compounds to form embodiments not specifically set forth above. In addition, in the event that a list of substituents and/or variables is listed for any particular group or variable, it is understood that each individual substituent and/or variable may be deleted from the particular embodiment and/or claim and that the remaining list of substituents and/or variables will be considered to be within the scope of embodiments provided herein.

It is understood that in the present description, combinations of substituents and/or variables of the depicted formulae are permissible only if such contributions result in stable compounds.

In one embodiment, the phospholipid is 1,2-distearoyl-sn-glycero-3-phosphocholine (DSPC). In one embodiment, the phospholipid is 1,2-dioleoyl-sn-glycero-3-phosphoethanolamine (DOPE). In one embodiment, the phospholipid is 1,2-dilinoleoyl-sn-glycero-3-phosphocholine (DLPC). In one embodiment, the phospholipid is 1,2-dimyristoyl-sn-glycero-phosphocholine (DMPC). In one embodiment, the phospholipid is 1,2-dioleoyl-sn-glycero-3-phosphocholine (DOPC). In one embodiment, the phospholipid is 1,2-dipalmitoyl-sn-glycero-3-phosphocholine (DPPC). In one embodiment, the phospholipid is 1,2-diundecanoyl-sn-glycero-phosphocholine (DUPC). In one embodiment, the phospholipid is 1-palmitoyl-2-oleoyl-sn-glycero-3-phosphocholine (POPC).

In one embodiment, the lipid nanoparticle comprises no more than 0.5 mol percent phospholipid. In one embodiment, the lipid nanoparticle comprises no more than 0.01 mol percent phospholipid, no more than 0.05 mol percent phospholipid, no more than 0.1 mol percent phospholipid, no more than 0.15 mol percent phospholipid, no more than 0.2 mol percent phospholipid, no more than 0.25 mol percent phospholipid, no more than 0.3 mol percent phospholipid, no more than 0.35 mol percent phospholipid, no more than 0.4 mol percent phospholipid, no more than 0.45 mol percent phospholipid or no more than 0.5 mol percent phospholipid. In one embodiment, the lipid nanoparticle does not comprise phospholipid.

In one embodiment, the lipid nanoparticle has a lipid:nucleic acid mass ratio of from about 9:1 to about 20:1. In one embodiment, the lipid nanoparticle has a lipid:nucleic acid mass ratio of from about 9:1 to about 15:1. In one embodiment, the lipid nanoparticle has a lipid:nucleic acid mass ratio of from about 9:1 to about 12:1. In one embodiment, the lipid:nucleic acid mass ratio is about 9:1. In one embodiment, the lipid:nucleic acid mass ratio is about 10:1. In one embodiment, the lipid:nucleic acid mass ratio is about 11:1. In one embodiment, the lipid:nucleic acid mass ratio is about 12:1. In one embodiment, the lipid:nucleic acid mass ratio is about 13:1. In one embodiment, the lipid:nucleic acid mass ratio is about 14:1. In one embodiment, the lipid:nucleic acid mass ratio is about 15:1. In one embodiment, the lipid:nucleic acid mass ratio is about 16:1. In one embodiment, the lipid:nucleic acid mass ratio is about 17:1. In one embodiment, the lipid:nucleic acid mass ratio is about 18:1. In one embodiment, the lipid:nucleic acid mass ratio is about 19:1. In one embodiment, the lipid:nucleic acid mass ratio is about 20:1.

In one embodiment, the size and encapsulation efficiency of the LNPs provided herein are comparable to those of conventional LNP formulations. In one embodiment, the LNPs provided herein have improved delivery efficiency of mRNA in vitro and/or in vivo as evaluated by protein expression level as compared with the conventional LNP formulations.

In one embodiment, LNPs are vesicles including one or more lipid bilayers. In one embodiment, an LNP includes two or more concentric bilayers separated by aqueous compartments. Lipid bilayers may be functionalized and/or crosslinked to one another. Lipid bilayers may include one or more ligands, proteins, or channels.

In one embodiment, the nanoparticle comprises one or more nucleic acid molecules as the therapeutic payload.

In one embodiment, the nucleic acid is an RNA. In one embodiment, the nucleic acid is a messenger RNA (mRNA), small interfering RNA (siRNA), asymmetrical interfering RNA (aiRNA), microRNA (miRNA), dicer-substrate RNA (dsRNA), small hairpin RNA (shRNA), transfer RNA (tRNA), small guide RNA (sgRNA), Cas9 RNA, or a mixture thereof. In one embodiment, the nucleic acid is an mRNA. In one embodiment, the nucleic acid is a DNA. In one embodiment, the nucleic acid is a plasmid DNA. In one embodiment, the nucleic acid is a supercoiled DNA. In one embodiment, the nucleic acid is a linear DNA.

In one embodiment, the mRNA is an mRNA that encodes an antigen or a fragment or epitope thereof. In one embodiment, the mRNA is an mRNA that encodes a pathogenic antigen. In one embodiment, the mRNA is an mRNA that encodes a tumor associated antigen. In one embodiment, the mRNA is an mRNA that encodes a tumor specific antigen.

In one embodiment, the mRNA is monocistronic mRNA. In one embodiment, the mRNA is multicistronic mRNA. In one embodiment, the mRNA is a multicistronic mRNA comprising two or more open reading frames (ORFs). In one embodiment, the multicistronic mRNA encodes two identical peptides or proteins. In one embodiment, the multicistronic mRNA encodes two different peptides or proteins.

In one embodiment, each peptide or protein encoded by a multicistronic mRNA comprises at least one epitope of a selected antigen. In one embodiment, different peptide or protein encoded by a multicistronic mRNA each comprises at least one epitope of different antigens.

In one embodiment, the at least one epitope can be at least 2, at least 3, at least 4, at least 5, at least 6, at least 7, at least 8, at least 9, or at least 10 epitopes of an antigen.

In one embodiment, the nucleic acid is a small interfering RNA (siRNA).

In one embodiment, the siRNA selectively silences a gene associated with a particular disease, disorder, or condition upon administration to a subject in need thereof of a nanoparticle composition including the siRNA.

In one embodiment, the nucleic acid is an immunomodulatory siRNA.

In one embodiment, the siRNA comprises a sequence that is complementary to an mRNA sequence encoding a protein product of interest.

In one embodiment, the nucleic acid molecule contains only canonical nucleotides selected from A (adenosine), G (guanosine), C (cytosine), U (uridine), and T (thymidine). In one embodiment, the nucleic acid comprises at least one functional nucleotide analog. In one embodiment, a functional nucleotide analog contains a non-canonical nucleobase.

In one embodiment, the nucleic acid is chemically modified. In one embodiment, the nucleic acid comprises at least one chemical modification to the nucleobase. Exemplary modification to nucleobases includes but are not limited to one or more substitutions or modifications including but not limited to alkyl, aryl, halo, oxo, hydroxyl, alkyloxy, and/or thio substitutions; one or more fused or open rings, oxidation, and/or reduction. In one embodiment, the nucleic acid comprises at least one chemical modification to the sugar groups. In one embodiment, the nucleic acid comprises at least one chemical modification to internucleoside linkage.

In one aspect, nucleic acid molecules described herein are formulated for in vitro and in vivo gene delivery. Particularly, in one embodiment the nucleic acid molecule is formulated into a lipid nanoparticle. In one embodiment, the lipid nanoparticles encapsulate the nucleic acid molecule within the lipid shell. In one embodiment, the lipid shells protect the nucleic acid molecules from degradation. In one embodiment, the lipid nanoparticles also facilitate transportation of the enclosed nucleic acid molecules into intracellular compartments and/or machinery to exert an intended therapeutic or prophylactic function. In certain embodiments, nucleic acids, when present in the lipid nanoparticles, are resistant in aqueous solution against nuclease degradation. Lipid nanoparticles comprising nucleic acids and their method of preparation are known in the art, such as those disclosed in, e.g., U.S. Patent Publication No. 2004/0142025, U.S. Patent Publication No. 2007/0042031, PCT Publication No. WO 2017/004143, PCT Publication No. WO 2015/199952, PCT Publication No. WO 2013/016058, and PCT Publication No. WO 2013/086373, the full disclosures of each of which are herein incorporated by reference in their entirety for all purposes.

In one embodiment, the largest dimension of a LNP composition provided herein is 1 μm or shorter (e.g., ≤1 μm, ≤900 nm, ≤800 nm, ≤700 nm, ≤600 nm, ≤500 nm, ≤400 nm, ≤300 nm, ≤200 nm, ≤175 nm, ≤150 nm, ≤125 nm, ≤100 nm, ≤75 nm, ≤50 nm, or shorter), such as when measured by dynamic light scattering (DLS), transmission electron microscopy, scanning electron microscopy, or another method.

In one embodiment, provided herein is a lipid nanoparticle, wherein the size of the nanoparticle is from about 40 nm to about 150 nm. In one embodiment, the size of the nanoparticle is from about 50 nm to about 100 nm. In one embodiment, the size of the nanoparticle is from about 40 nm to about 120 nm. In one embodiment, the size of the nanoparticle is from about 40 nm to about 100 nm. In one embodiment, the size of the nanoparticle is from about 50 nm to about 150 nm. In one embodiment, the size of the nanoparticle is from about 60 nm to about 150 nm. In one embodiment, the size of the nanoparticle is from about 60 nm to about 120 nm. In one embodiment, the size of the nanoparticle is from about 70 nm to about 120 nm. In one embodiment, the size of the nanoparticle is from about 80 nm to about 120 nm.

As used herein and unless otherwise specified, the size of a nanoparticle refers to the mean size of a population or a plural of said nanoparticles. In one embodiment, the size of a three-lipid-component lipid nanoparticle provided herein (with no phospholipid component, or reduced/insignificant amount of phospholipid component) is increased as compared to a reference formulation; wherein the reference formulation differs from the lipid nanoparticle in that (i) the reference formulation comprises a phospholipid, and (ii) the molar ratio of the cationic lipid:the steroid:the phospholipid:the polymer conjugated lipid is about 50:38.5:10:1.5. In one embodiment, a three-lipid-component nanoparticle provided herein has an increase in size of at least about 30%, at least about 40%, at least about 50%, at least about 60%, at least about 70%, or at least about 80%. In one embodiment, the size of the nanoparticle is increased by at least about 50% as compared to the reference formulation. In one embodiment, a three-lipid-component nanoparticle provided herein has an increase in size of no more than about 90%, no more than about 100%, or no more than about 110%. In one embodiment, the size (of a three-lipid-component nanoparticle provided herein) is from about 90 nm to about 150 nm. In one embodiment, the size is from about 100 nm to about 140 nm. In one embodiment, the size is from about 110 nm to about 130 nm. In one embodiment, the size is from about 100 nm to about 110 nm. In one embodiment, the size is from about 110 nm to about 120 nm. In one embodiment, the size is from about 120 nm to about 130 nm.

In one embodiment, the size of the nanoparticle is about 40 nm. In one embodiment, the size of the nanoparticle is about 50 nm. In one embodiment, the size of the nanoparticle is about 55 nm. In one embodiment, the size of the nanoparticle is about 60 nm. In one embodiment, the size of the nanoparticle is about 65 nm. In one embodiment, the size of the nanoparticle is about 70 nm. In one embodiment, the size of the nanoparticle is about 75 nm. In one embodiment, the size of the nanoparticle is about 80 nm. In one embodiment, the size of the nanoparticle is about 85 nm. In one embodiment, the size of the nanoparticle is about 90 nm. In one embodiment, the size of the nanoparticle is about 95 nm. In one embodiment, the size of the nanoparticle is about 100 nm. In one embodiment, the size of the nanoparticle is about 105 nm. In one embodiment, the size of the nanoparticle is about 110 nm. In one embodiment, the size of the nanoparticle is about 115 nm. In one embodiment, the size of the nanoparticle is about 120 nm. In one embodiment, the size of the nanoparticle is about 125 nm. In one embodiment, the size of the nanoparticle is about 125 nm. In one embodiment, the size of the nanoparticle is about 130 nm. In one embodiment, the size of the nanoparticle is about 135 nm. In one embodiment, the size of the nanoparticle is about 140 nm. In one embodiment, the size of the nanoparticle is about 145 nm. In one embodiment, the size of the nanoparticle is about 150 nm. In one embodiment, the size of the nanoparticle is about 155 nm.

In one embodiment, a lipid nanoparticle provided herein has an encapsulation efficiency of the nucleic acid of at least about 80%. In one embodiment, a lipid nanoparticle provided herein has an encapsulation efficiency of mRNA at least about 80%. In one embodiment, a lipid nanoparticle provided herein has an encapsulation efficiency of DNA at least about 80%. In one embodiment, a lipid nanoparticle provided herein has an encapsulation efficiency of siRNA at least about 80%.

In one embodiment, a lipid nanoparticle provided herein has an encapsulation efficiency of the nucleic acid of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, or at least about 96%.

In one embodiment, a lipid nanoparticle provided herein has an encapsulation efficiency of mRNA of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, or at least about 96%.

In one embodiment, the nanoparticle has an encapsulation efficiency of the nucleic acid of at least about 90%. In one embodiment, the nanoparticle has an encapsulation efficiency of mRNA of at least about 90%. In one embodiment, the nanoparticle has an encapsulation efficiency of DNA of at least about 90%. In one embodiment, the nanoparticle has an encapsulation efficiency of siRNA of at least about 90%.

In one embodiment, the nucleic acid encodes a RNA or protein, wherein the amount of RNA or protein expressed from the nucleic acid in the nanoparticle in a mammalian cell or a tissue of a mammal is more than the amount of RNA or protein expressed from the nucleic acid in a reference formulation; wherein the reference formulation differs from the lipid nanoparticle in that (i) the reference formulation comprises a phospholipid, and (ii) the molar ratio of the cationic lipid:the steroid:the phospholipid:the polymer conjugated lipid is about 50:38.5:10:1.5.

In one embodiment, the amount of phospholipid in the reference formulation is more than 5 mol % of the total lipid present in the reference formulation. In one embodiment, the amount of phospholipid in the reference formulation is more than 10 mol % of the total lipid present in the reference formulation. In one embodiment, the amount of phospholipid in the reference formulation is more than 15 mol % of the total lipid present in the reference formulation. In one embodiment, the amount of phospholipid in the reference formulation is more than 20 mol % of the total lipid present in the reference formulation. In one embodiment, the amount of phospholipid in the reference formulation is more than 25 mol % of the total lipid present in the reference formulation. In one embodiment, the amount of phospholipid in the reference formulation is more than 30 mol % of the total lipid present in the reference formulation. In one embodiment, the amount of phospholipid in the reference formulation is more than 35 mol % of the total lipid present in the reference formulation. In one embodiment, the amount of phospholipid in the reference formulation is more than 40 mol % of the total lipid present in the reference formulation. In one embodiment, the amount of phospholipid in the reference formulation is more than 45 mol % of the total lipid present in the reference formulation. In one embodiment, the amount of phospholipid in the reference formulation is more than 50 mol % of the total lipid present in the reference formulation.

In one embodiment, the amount of phospholipid in the reference formulation is about 10 mol % of the total lipid present in the reference formulation.

In one embodiment, the reference formulation comprises DSPC. In one embodiment, the reference formulation comprises DOPE.

In one embodiment, the amount of RNA or protein expressed from the nucleic acid in a nanoparticle provided herein is at least about 50% more than the amount of RNA or protein expressed from the nucleic acid in a reference formulation.

In one embodiment, the amount of RNA or protein expressed from the nucleic acid in a nanoparticle provided herein is at least about 55%, at least about 60%, at least about 65%, at least about 70%, at least about 75%, at least about 80%, at least about 85%, at least about 90%, at least about 95% or at least about 100% more than the amount of RNA or protein expressed from the nucleic acid in a reference formulation.

In one embodiment, the amount of RNA or protein expressed from the nucleic acid in a nanoparticle provided herein is at least about 100% more than the amount of RNA or protein expressed from the nucleic acid in a reference formulation. In one embodiment, the amount of RNA or protein expressed from the nucleic acid in a nanoparticle provided herein is at least about 150% more than the amount of RNA or protein expressed from the nucleic acid in a reference formulation. In one embodiment, the amount of RNA or protein expressed from the nucleic acid in a nanoparticle provided herein is at least about 200% more than the amount of RNA or protein expressed from the nucleic acid in a reference formulation. In one embodiment, the amount of RNA or protein expressed from the nucleic acid in a nanoparticle provided herein is at least about 250% more than the amount of RNA or protein expressed from the nucleic acid in a reference formulation. In one embodiment, the amount of RNA or protein expressed from the nucleic acid in a nanoparticle provided herein is at least about 300% more than the amount of RNA or protein expressed from the nucleic acid in a reference formulation.

In one embodiment, the amount of RNA or protein expressed from the nucleic acid in a nanoparticle provided herein is at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340% or at least about 350% more than the amount of RNA or protein expressed from the nucleic acid in a reference formulation.

In one embodiment, provided herein is a pharmaceutical composition comprising a lipid nanoparticle provided herein, and a pharmaceutically acceptable excipient. In one embodiment, an excipient is approved for use in humans and for veterinary use.

In one embodiment, one or more excipients can make up greater than 50% of the total mass or volume of a pharmaceutical composition comprising the nanoparticle composition. For example, the one or more excipients can make up about 50%, 60%, 70%, 80%, 90%, or more of a pharmaceutical composition.

In one embodiment, a pharmaceutical composition can comprise between 0.1% and 100% (wt/wt) of one or more nanoparticle compositions.

In one embodiment, a pharmaceutical composition comprises 100% (wt/wt) of the nanoparticle compositions. In one embodiment, a pharmaceutical composition comprises about 75% (wt/wt) of the nanoparticle compositions. In one embodiment, a pharmaceutical composition comprises about 50% (wt/wt) of the nanoparticle compositions. In one embodiment, a pharmaceutical composition comprises about 25% (wt/wt) of the nanoparticle compositions. In one embodiment, a pharmaceutical composition comprises 1% (wt/wt) of the nanoparticle compositions. In one embodiment, a pharmaceutical composition comprises 0.1% (wt/wt) of the nanoparticle compositions.

In one embodiment, provided herein is a method of expressing an mRNA in a mammalian cell or a tissue of a mammal comprising delivering a lipid nanoparticle provided herein or a pharmaceutical composition provided herein, wherein the nucleic acid is an mRNA, to the mammalian cells or the tissue of the mammal; and wherein the delivered mRNA is expressed in the mammalian cell or in the tissue of the mammal.

In one embodiment, provided herein is a method for introducing an mRNA that encodes a protein into a cell, comprising contacting the cell with a lipid nanoparticle provided herein or a pharmaceutical composition provided herein.

In one embodiment, the cell is a prokaryotic cell. In one embodiment, the cell is a eukaryotic cell. In one embodiment, the cell is in a mammal. In one embodiment, the cell is a mammalian cell. In one embodiment, the cell is in a human.

In one embodiment, the cell is in liver. In one embodiment, the cell is in lung. In one embodiment, the cell is in spleen. In one embodiment, the cell is in colon. In one embodiment, the cell is in kidney.

In one embodiment, the cell is an immune cell. Examples of immune cells include but are not limited to T-cells, B-cells, natural killer cells, neutrophils, monocytes, and macrophages.

In one embodiment, the cell is a genetically engineered cell.

In one embodiment, the cell is a stem cell. Examples of stem cells include but are not limited to pluripotent stem cell, mesenchymal stem cell, neural stem cell and hematopoietic stem cell et al.

In one embodiment, the cell is a cancer cell.

In one embodiment, provided herein is a method for treating a disease or disorder in a human, comprising administering to the human a therapeutically effective amount of a lipid nanoparticle provided herein or a pharmaceutical composition provided herein.

In one embodiment, provided herein is a method for treating a disease or disorder caused by impaired expression of a protein in a human, comprising administering to the human a therapeutically effective amount of a lipid nanoparticle provided herein or a pharmaceutical composition provided herein, wherein the nucleic acid is an mRNA that encodes the protein. In one embodiment, the expression level of the protein is increased by at least 10%, at least 20%, at least 40%, at least 60%, at least 80%, at least 100%, at least 150%, at least 200%, at least 300%, at least 400%, at least 500%, at least 750% or at least 1000%.

In one embodiment, provided herein is a method for treating a disease or disorder caused by overexpression of a protein in a human, comprising administering to the human a therapeutically effective amount of a lipid nanoparticle provided herein or a pharmaceutical composition provided herein, wherein the nucleic acid is an siRNA that reduces the expression level of the protein. In one embodiment, the expression level of the protein is reduced by 10%, 20%, 30%, 40%, 50%, 60%, 70%, 80%, 90%, 95% or 99%.

In one embodiment, the diseases or disorder is a viral infection. In one embodiment, the diseases or disorder is a liver disease. In one embodiment, the diseases or disorder is a cancer.

In one embodiment, the administration is intranasal, intravenous, intraperitoneal, intramuscular, intraarticular, intralesional, intratracheal, subcutaneous, or intradermal administration. In one embodiment, the administration is intravenous administration. In one embodiment, the administration is intramuscular administration. In one embodiment, the administration is oral administration.

In one embodiment, provided herein is a method for preventing a disease in a subject by administering to the subject a vaccine comprising a therapeutically effective amount of a lipid nanoparticle provided herein or a pharmaceutical composition provided herein, wherein the nucleic acid encodes an antigen associated with the disease thereby triggering an immune response against the target disease in the subject. In one embodiment, the immune response is adaptive immune response or innate immune response or both.

In one embodiment, the vaccine is administered before the onset of clinical symptoms of the target disease, thereby renders the vaccinated human immune or less susceptible to the development of the said disease. In one embodiment, the vaccine is administered after the onset of clinical symptoms, thereby alleviate the symptoms and/or prevent reoccurrence.

In one embodiment, the antigen is virus. In one embodiment, the antigen is bacteria. In one embodiment, the antigen is fungi. In one embodiment, the antigen is parasite.

In one embodiment, the pathogen is a coronavirus (e.g., SARS, SARS-Cov-2, MERS), influenza, measles, human papillomavirus (HPV), rabies, meningitis, whooping cough, tetanus, plague, hepatitis, or tuberculosis.

In one embodiment, the therapeutic payload comprises a nucleic acid sequence (e.g., mRNA) encoding a pathogenic protein characteristic for the pathogen, or an antigenic fragment or epitope thereof. The vaccine, upon administration to a vaccinated subject, allows for expression of the encoded pathogenic protein (or the antigenic fragment or epitope thereof), thereby eliciting immunity in the subject against the pathogen.

In one embodiment, provided herein is a method for preventing infectious diseases in a subject by administering to the subject a vaccine comprising a therapeutically effective amount of a lipid nanoparticle provided herein or a pharmaceutical composition provided herein, wherein the nucleic acid encodes a polypeptide associated with the infectious disease thereby triggering an immune response against the target disease in human.

In one embodiment, the infectious disease is virus infection. In one embodiment, the infectious disease is bacterial infection.

In one embodiment, the nucleic acid is mRNA that encodes polypeptide present in the antigen.

### 6.3.1 Therapeutic Payload

Nanoparticle compositions as described herein can comprise one or more therapeutic and/or prophylactic agents. These therapeutic and/or prophylactic agents are sometimes referred to as a “therapeutic payload” or “payload” in the present disclosure. In some embodiments, the therapeutic payload can be administered in vivo or in vitro using the nanoparticles as a delivery vehicle.

In one embodiment, the therapeutic payload is the nucleic acid provided herein. In one embodiment, the nanoparticle composition comprises a therapeutic payment in addition to the nucleic acid provided herein. In one embodiment, the nanoparticle composition comprises a therapeutic payment in place of the nucleic acid provided herein.

In some embodiments, the nanoparticle composition comprises, as the therapeutic payload, a small molecule compound (e.g., a small molecule drug) such as antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cytosine arabinoside, anthracyclines, alkylating agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetalol), antihypertensive agents (e.g., clonidine and hydralazine), anti-depressants (e.g., imipramine, amitriptyline, and doxepin), anti-convulsants (e.g., phenytoin), antihistamines (e.g., diphenhydramine, chlorpheniramine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

In some embodiments, the therapeutic payload comprises a cytotoxin, a radioactive ion, a chemotherapeutic, a vaccine, a compound that elicits an immune response, and/or another therapeutic and/or prophylactic agent. A cytotoxin or cytotoxic agent includes any agent that may be detrimental to cells. Examples include, but are not limited to, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, e.g., maytansinol, rachelmycin (CC-1065), and analogs or homologs thereof. Radioactive ions include, but are not limited to iodine (e.g., iodine 125 or iodine 131), strontium 89, phosphorous, palladium, cesium, iridium, phosphate, cobalt, yttrium 90, samarium 153, and praseodymium.

In other embodiments, the therapeutic payload of the present nanoparticle composition can include, but is not limited to, therapeutic and/or prophylactic agents such as antimetabolites (e.g., methotrexate, 6-mercaptopurine, 6-thioguanine, cytarabine, 5-fluorouracil, dacarbazine), alkylating agents (e.g., mechlorethamine, thiotepa chlorambucil, rachelmycin (CC-1065), melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In some embodiments, the nanoparticle composition comprises, as the therapeutic payload, a biological molecule such as peptides and polypeptides. The biological molecules forming part of the present nanoparticle composition can be either of a natural source or synthetic. For example, in some embodiments, the therapeutic payload of the present nanoparticle composition can include, but is not limited to gentamycin, amikacin, insulin, erythropoietin (EPO), granulocyte-colony stimulating factor (G-CSF), granulocyte-macrophage colony stimulating factor (GM-CSF), Factor VIR, luteinizing hormone-releasing hormone (LHRH) analogs, interferons, heparin, Hepatitis B surface antigen, typhoid vaccine, cholera vaccine, and peptides and polypeptides.

### 6.3.1.1 Additional Description of Nucleic Acids

In some embodiments, the present nanoparticle composition comprises one or more nucleic acid molecules (e.g., DNA or RNA molecules) as the therapeutic payload. Exemplary forms of nucleic acid molecules that can be included in the present nanoparticle composition as therapeutic payload include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In certain embodiments, the therapeutic payload comprises an RNA. RNA molecules that can be included in the present nanoparticle composition as the therapeutic payload include, but are not limited to, shortmers, agomirs, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and other forms of RNA molecules known in the art. In particular embodiments, the RNA is an mRNA.

In one embodiment, the present nanoparticle composition comprises one or more nucleic acid molecules (e.g., DNA or RNA molecules) as the therapeutic payload. Exemplary forms of nucleic acid molecules that can be included in the present nanoparticle composition as therapeutic payload include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In certain embodiments, the therapeutic payload comprises an RNA. RNA molecules that can be included in the present nanoparticle composition as the therapeutic payload include, but are not limited to, shortmers, agomirs, antagomirs, antisense, ribozymes, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and other forms of RNA molecules known in the art. In particular embodiments, the RNA is an mRNA.

In other embodiments, the nanoparticle composition comprises a siRNA molecule as the therapeutic payload. Particularly, in one embodiment, the siRNA molecule is capable of selectively interfering with and downregulate the expression of a gene of interest. For example, in one embodiment, the siRNA payload selectively silence a gene associated with a particular disease, disorder, or condition upon administration to a subject in need thereof of a nanoparticle composition including the siRNA. In one embodiment, the siRNA molecule comprises a sequence that is complementary to an mRNA sequence encoding a protein product of interest. In one embodiment, the siRNA molecule is an immunomodulatory siRNA.

In one embodiment, the nanoparticle composition comprises a shRNA molecule or a vector encoding the shRNA molecule as the therapeutic payload. Particularly, in one embodiment, the therapeutic payload, upon administering to a target cell, produces shRNA inside the target cell. Constructs and mechanisms relating to shRNA are well known in the relevant arts.

In one embodiment, the nanoparticle composition comprises an mRNA molecule as the therapeutic payload. Particularly, in one embodiment, the mRNA molecule encodes a polypeptide of interest, including any naturally or non-naturally occurring or otherwise modified polypeptide. A polypeptide encoded by an mRNA may be of any size and may have any secondary structure or activity. In one embodiment, the polypeptide encoded by an mRNA payload can have a therapeutic effect when expressed in a cell.

In some embodiment, a nucleic acid molecule of the present disclosure comprises an mRNA molecule. In specific embodiments, the nucleic acid molecule comprises at least one coding region encoding a peptide or polypeptide of interest (e.g., an open reading frame (ORF)). In one embodiment, the nucleic acid molecule further comprises at least one untranslated region (UTR). In particular embodiments, the untranslated region (UTR) is located upstream (to the 5′-end) of the coding region and is referred to herein as the 5′-UTR. In particular embodiments, the untranslated region (UTR) is located downstream (to the 3′-end) of the coding region and is referred to herein as the 3′-UTR. In particular embodiments, the nucleic acid molecule comprises both a 5′-UTR and a 3′-UTR. In one embodiment, the 5′-UTR comprises a 5′-Cap structure. In one embodiment, the nucleic acid molecule comprises a Kozak sequence (e.g., in the 5′-UTR). In one embodiment, the nucleic acid molecule comprises a poly-A region (e.g., in the 3′-UTR). In one embodiment, the nucleic acid molecule comprises a polyadenylation signal (e.g., in the 3′-UTR). In one embodiment, the nucleic acid molecule comprises stabilizing region (e.g., in the 3′-UTR). In one embodiment, the nucleic acid molecule comprises a secondary structure. In one embodiment, the secondary structure is a stem-loop. In one embodiment, the nucleic acid molecule comprises a stem-loop sequence (e.g., in the 5′-UTR and/or the 3′-UTR). In one embodiment, the nucleic acid molecule comprises one or more intronic regions capable of being excised during splicing. In a specific embodiment, the nucleic acid molecule comprises one or more region selected from a 5′-UTR, and a coding region. In a specific embodiment, the nucleic acid molecule comprises one or more region selected from a coding region and a 3′-UTR. In a specific embodiment, the nucleic acid molecule comprises one or more region selected from a 5′-UTR, a coding region, and a 3′-UTR.

### Coding Region

In one embodiment, the nucleic acid molecule of the present disclosure comprises at least one coding region. In one embodiment, the coding region is an open reading frame (ORF) that encodes for a single peptide or protein. In one embodiment, the coding region comprises at least two ORFs, each encoding a peptide or protein. In those embodiments where the coding region comprises more than one ORFs, the encoded peptides and/or proteins can be the same as or different from each other. In one embodiment, the multiple ORFs in a coding region are separated by non-coding sequences. In specific embodiments, a non-coding sequence separating two ORFs comprises an internal ribosome entry sites (IRES).

Without being bound by the theory, it is contemplated that an internal ribosome entry sites (IRES) can act as the sole ribosome binding site, or serve as one of multiple ribosome binding sites of an mRNA. An mRNA molecule containing more than one functional ribosome binding site can encode several peptides or polypeptides that are translated independently by the ribosomes (e.g., multicistronic mRNA). Accordingly, in one embodiment, the nucleic acid molecule of the present disclosure (e.g., mRNA) comprises one or more internal ribosome entry sites (IRES). Examples of IRES sequences that can be used in connection with the present disclosure include, without limitation, those from picomaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In various embodiments, the nucleic acid molecule of the present disclose encodes for at least 1, 2, 3, 4, 5, 6, 7, 8, 9, 10 or more than 10 peptides or proteins. Peptides and proteins encoded by a nucleic acid molecule can be the same or different. In one embodiment, the nucleic acid molecule of the present disclosure encodes a dipeptide (e.g., camosine and anserine). In one embodiment, the nucleic acid molecule encodes a tripeptide. In one embodiment, the nucleic acid molecule encodes a tetrapeptide. In one embodiment, the nucleic acid molecule encodes a pentapeptide. In one embodiment, the nucleic acid molecule encodes a hexapeptide. In one embodiment, the nucleic acid molecule encodes a heptapeptide. In one embodiment, the nucleic acid molecule encodes an octapeptide. In one embodiment, the nucleic acid molecule encodes a nonapeptide. In one embodiment, the nucleic acid molecule encodes a decapeptide. In one embodiment, the nucleic acid molecule encodes a peptide or polypeptide that has at least about 15 amino acids. In one embodiment, the nucleic acid molecule encodes a peptide or polypeptide that has at least about 50 amino acids. In one embodiment, the nucleic acid molecule encodes a peptide or polypeptide that has at least about 100 amino acids. In one embodiment, the nucleic acid molecule encodes a peptide or polypeptide that has at least about 150 amino acids. In one embodiment, the nucleic acid molecule encodes a peptide or polypeptide that has at least about 300 amino acids. In one embodiment, the nucleic acid molecule encodes a peptide or polypeptide that has at least about 500 amino acids. In one embodiment, the nucleic acid molecule encodes a peptide or polypeptide that has at least about 1000 amino acids.

In one embodiment, the nucleic acid molecule of the present disclosure is at least about 30 nucleotides (nt) in length. In one embodiment, the nucleic acid molecule is at least about 35 nt in length. In one embodiment, the nucleic acid molecule is at least about 40 nt in length. In one embodiment, the nucleic acid molecule is at least about 45 nt in length. In one embodiment the nucleic acid molecule is at least about 50 nt in length. In one embodiment, the nucleic acid molecule is at least about 55 nt in length. In one embodiment, the nucleic acid molecule is at least about 60 nt in length. In one embodiment, the nucleic acid molecule is at least about 65 nt in length. In one embodiment, the nucleic acid molecule is at least about 70 nt in length. In one embodiment, the nucleic acid molecule is at least about 75 nt in length. In one embodiment, the nucleic acid molecule is at least about 80 nt in length. In one embodiment the nucleic acid molecule is at least about 85 nt in length. In one embodiment, the nucleic acid molecule is at least about 90 nt in length. In one embodiment, the nucleic acid molecule is at least about 95 nt in length. In one embodiment, the nucleic acid molecule is at least about 100 nt in length. In one embodiment, the nucleic acid molecule is at least about 120 nt in length. In one embodiment, the nucleic acid molecule is at least about 140 nt in length. In one embodiment, the nucleic acid molecule is at least about 160 nt in length. In one embodiment, the nucleic acid molecule is at least about 180 nt in length. In one embodiment, the nucleic acid molecule is at least about 200 nt in length. In one embodiment, the nucleic acid molecule is at least about 250 nt in length. In one embodiment, the nucleic acid molecule is at least about 300 nt in length. In one embodiment, the nucleic acid molecule is at least about 400 nt in length. In one embodiment, the nucleic acid molecule is at least about 500 nt in length. In one embodiment, the nucleic acid molecule is at least about 600 nt in length. In one embodiment, the nucleic acid molecule is at least about 700 nt in length. In one embodiment, the nucleic acid molecule is at least about 800 nt in length. In one embodiment, the nucleic acid molecule is at least about 900 nt in length. In one embodiment, the nucleic acid molecule is at least about 1000 nt in length. In one embodiment, the nucleic acid molecule is at least about 1100 nt in length. In one embodiment, the nucleic acid molecule is at least about 1200 nt in length. In one embodiment, the nucleic acid molecule is at least about 1300 nt in length. In one embodiment, the nucleic acid molecule is at least about 1400 nt in length. In one embodiment, the nucleic acid molecule is at least about 1500 nt in length. In one embodiment, the nucleic acid molecule is at least about 1600 nt in length. In one embodiment, the nucleic acid molecule is at least about 1700 nt in length. In one embodiment, the nucleic acid molecule is at least about 1800 nt in length. In one embodiment, the nucleic acid molecule is at least about 1900 nt in length. In one embodiment, the nucleic acid molecule is at least about 2000 nt in length. In one embodiment, the nucleic acid molecule is at least about 2500 nt in length. In one embodiment, the nucleic acid molecule is at least about 3000 nt in length. In one embodiment, the nucleic acid molecule is at least about 3500 nt in length. In one embodiment, the nucleic acid molecule is at least about 4000 nt in length. In one embodiment, the nucleic acid molecule is at least about 4500 nt in length. In one embodiment, the nucleic acid molecule is at least about 5000 nt in length.

In specific embodiments, the therapeutic payload comprises a vaccine composition (e.g., a genetic vaccine) as described herein. In one embodiment, the therapeutic payload comprises a compound capable of triggering immunity against one or more target conditions or disease. In one embodiment, the target condition is related to or caused by infection by a pathogen, such as a coronavirus (e.g. 2019-nCoV), influenza, measles, human papillomavirus (HPV), rabies, meningitis, whooping cough, tetanus, plague, hepatitis, and tuberculosis. In one embodiment, the therapeutic payload comprises a nucleic acid sequence (e.g., mRNA) encoding a pathogenic protein characteristic for the pathogen, or an antigenic fragment or epitope thereof. The vaccine, upon administration to a vaccinated subject, allows for expression of the encoded pathogenic protein (or the antigenic fragment or epitope thereof), thereby eliciting immunity in the subject against the pathogen.

In one embodiment, the target condition is related to or caused by neoplastic growth of cells, such as a cancer. In one embodiment, the therapeutic payload comprises a nucleic acid sequence (e.g., mRNA) encoding a tumor associated antigen (TAA) characteristic for the cancer, or an antigenic fragment or epitope thereof. The vaccine, upon administration to a vaccinated subject, allows for expression of the encoded TAA (or the antigenic fragment or epitope thereof), thereby eliciting immunity in the subject against the neoplastic cells expressing the TAA.

### 5′-Cap Structure

Without being bound by the theory, it is contemplated that, a 5′-cap structure of a polynucleotide is involved in nuclear export and increasing polynucleotide stability and binds the mRNA Cap Binding Protein (CBP), which is responsible for polynucleotide stability in the cell and translation competency through the association of CBP with poly-A binding protein to form the mature cyclic mRNA species. The 5′-cap structure further assists the removal of 5′-proximal introns removal during mRNA splicing. Accordingly, in one embodiment, the nucleic acid molecules of the present disclosure comprise a 5′-cap structure.

Nucleic acid molecules may be 5′-end capped by the endogenous transcription machinery of a cell to generate a 5′-ppp-5′-triphosphate linkage between a terminal guanosine cap residue and the 5′-terminal transcribed sense nucleotide of the polynucleotide. This 5′-guanylate cap may then be methylated to generate an N7-methyl-guanylate residue. The ribose sugars of the terminal and/or anteterminal transcribed nucleotides of the 5′ end of the polynucleotide may optionally also be 2′-O-methylated. 5′-decapping through hydrolysis and cleavage of the guanylate cap structure may target a nucleic acid molecule, such as an mRNA molecule, for degradation.

In one embodiment, the nucleic acid molecules of the present disclosure comprise one or more alterations to the natural 5′-cap structure generated by the endogenous process. Without being bound by the theory, a modification on the 5′-cap may increase the stability of polynucleotide, increase the half-life of the polynucleotide, and could increase the polynucleotide translational efficiency.

Exemplary alterations to the natural 5′-Cap structure include generation of a non-hydrolyzable cap structure preventing decapping and thus increasing polynucleotide half-life. In one embodiment, because cap structure hydrolysis requires cleavage of 5′-ppp-5′ phosphorodiester linkages, in one embodiment, modified nucleotides may be used during the capping reaction. For example, in one embodiment, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used, such as α-methyl-phosphonate and seleno-phosphate nucleotides.

Additional exemplary alterations to the natural 5′-Cap structure also include modification at the 2′- and/or 3′-position of a capped guanosine triphosphate (GTP), a replacement of the sugar ring oxygen (that produced the carbocyclic ring) with a methylene moiety (CH₂), a modification at the triphosphate bridge moiety of the cap structure, or a modification at the nucleobase (G) moiety.

Additional exemplary alterations to the natural 5′-cap structure include, but are not limited to, 2′-O-methylation of the ribose sugars of 5′-terminal and/or 5′-anteterminal nucleotides of the polynucleotide (as mentioned above) on the 2′-hydroxy group of the sugar. Multiple distinct 5′-cap structures can be used to generate the 5′-cap of a polynucleotide, such as an mRNA molecule. Additional exemplary 5′-Cap structures that can be used in connection with the present disclosure further include those described in International Patent Publications: WO2008127688, WO 2008016473, and WO 2011015347, the entire contents of which are incorporated herein by reference.

In various embodiments, 5′-terminal caps can include cap analogs. Cap analogs, which herein are also referred to as synthetic cap analogs, chemical caps, chemical cap analogs, or structural or functional cap analogs, differ from natural (i.e., endogenous, wild-type, or physiological) 5′-caps in their chemical structure, and retaining cap function. Cap analogs may be chemically (i.e., non-enzymatically) or enzymatically synthesized and/linked to a polynucleotide.

For example, the Anti-Reverse Cap Analog (ARCA) cap contains two guanosines linked by a 5′-5′-triphosphate group, wherein one guanosine contains an N7-methyl group as well as a 3′-O-methyl group (i.e., N7,3′-O-dimethyl-guanosine-5′-triphosphate-5′-guanosine, m⁷G-3′mppp-G, which may equivalently be designated 3′ O-Me-m7G(5′)ppp(5′)G). The 3′-O atom of the other, unaltered, guanosine becomes linked to the 5′-terminal nucleotide of the capped polynucleotide (e.g., an mRNA). The N7- and 3′-O-methlyated guanosine provides the terminal moiety of the capped polynucleotide (e.g., mRNA). Another exemplary cap structure is mCAP, which is similar to ARCA but has a 2′-O-methyl group on guanosine (i.e., N7,2′-O-dimethyl-guanosine-5′-triphosphate-5′-guanosine, m₇Gm-ppp-G).

In one embodiment, a cap analog can be a dinucleotide cap analog. As a non-limiting example, the dinucleotide cap analog may be modified at different phosphate positions with a boranophosphate group or a phophoroselenoate group such as the dinucleotide cap analogs described in U.S. Pat. No. 8,519,110, the entire content of which is herein incorporated by reference in its entirety.

In one embodiment, a cap analog can be a N7-(4-chlorophenoxyethyl) substituted dinucleotide cap analog known in the art and/or described herein. Non-limiting examples of N7-(4-chlorophenoxyethyl) substituted dinucleotide cap analogs include a N7-(4-chlorophenoxyethyl)-G(5′)ppp(5′)G and a N7-(4-chlorophenoxyethyl)-m3′-OG(5′)ppp(5′)G cap analog (see, e.g., the various cap analogs and the methods of synthesizing cap analogs described in Kore et al. Bioorganic & Medicinal Chemistry 2013 21:4570-4574; the entire content of which is herein incorporated by reference). In other embodiments, a cap analog useful in connection with the nucleic acid molecules of the present disclosure is a 4-chloro/bromophenoxyethyl analog.

In various embodiments, a cap analog can include a guanosine analog. Useful guanosine analogs include but are not limited to inosine, N1-methyl-guanosine, 2′-fluoro-guanosine, 7-deaza-guanosine, 8-oxo-guanosine, 2-amino-guanosine, LNA-guanosine, and 2-azido-guanosine.

Without being bound by the theory, it is contemplated that while cap analogs allow for the concomitant capping of a polynucleotide in an in vitro transcription reaction, up to 20% of transcripts remain uncapped. This, as well as the structural differences of a cap analog from the natural 5′-cap structures of polynucleotides produced by the endogenous transcription machinery of a cell, may lead to reduced translational competency and reduced cellular stability.

Accordingly, in one embodiment, a nucleic acid molecule of the present disclosure can also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function, and/or structure as compared to synthetic features or analogs of the prior art, or which outperforms the corresponding endogenous, wild-type, natural, or physiological feature in one or more respects. Non-limiting examples of more authentic 5′-cap structures useful in connection with the nucleic acid molecules of the present disclosure are those which, among other things, have enhanced binding of cap binding proteins, increased half-life, reduced susceptibility to 5′-endonucleases, and/or reduced 5′-decapping, as compared to synthetic 5′-cap structures known in the art (or to a wild-type, natural or physiological 5′-cap structure). For example, in one embodiment, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanosine cap nucleotide wherein the cap guanosine contains an N7-methylation and the 5′-terminal nucleotide of the polynucleotide contains a 2′-O-methyl. Such a structure is termed the Cap1 structure. This cap results in a higher translational-competency, cellular stability, and a reduced activation of cellular pro-inflammatory cytokines, as compared, e.g., to other 5′cap analog structures known in the art. Other exemplary cap structures include 7mG(5′)ppp(5′)N,pN2p (Cap 0), 7mG(5′)ppp(5′)NlmpNp (Cap 1), 7mG(5′)-ppp(5′)NlmpN2mp (Cap 2), and m(7)Gpppm(3)(6,6,2′)Apm(2′)Apm(2′)Cpm(2)(3,2′)Up (Cap 4).

Without being bound by the theory, it is contemplated that the nucleic acid molecules of the present disclosure can be capped post-transcriptionally, and because this process is more efficient, nearly 100% of the nucleic acid molecules may be capped.

### Untranslated Regions (UTRs)

In one embodiment, the nucleic acid molecules of the present disclosure comprise one or more untranslated regions (UTRs). In one embodiment, an UTR is positioned upstream to a coding region in the nucleic acid molecule, and is termed 5′-UTR. In one embodiment, an UTR is positioned downstream to a coding region in the nucleic acid molecule, and is termed 3′-UTR. The sequence of an UTR can be homologous or heterologous to the sequence of the coding region found in a nucleic acid molecule. Multiple UTRs can be included in a nucleic acid molecule and can be of the same or different sequences, and/or genetic origin. According to the present disclosure, any portion of UTRs in a nucleic acid molecule (including none) can be codon optimized and any may independently contain one or more different structural or chemical modification, before and/or after codon optimization.

In one embodiment, a nucleic acid molecule of the present disclosure (e.g., mRNA) comprises UTRs and coding regions that are homologous with respect to each other. In other embodiments, a nucleic acid molecule of the present disclosure (e.g., mRNA) comprises UTRs and coding regions that are heterogeneous with respect to each other. In one embodiment, to monitor the activity of a UTR sequence, a nucleic acid molecule comprising the UTR and a coding sequence of a detectable probe can be administered in vitro (e.g., cell or tissue culture) or in vivo (e.g., to a subject), and an effect of the UTR sequence (e.g., modulation on the expression level, cellular localization of the encoded product, or half-life of the encoded product) can be measured using methods known in the art.

In one embodiment, the UTR of a nucleic acid molecule of the present disclosure (e.g., mRNA) comprises at least one translation enhancer element (TEE) that functions to increase the amount of polypeptide or protein produced from the nucleic acid molecule. In one embodiment, the TEE is located in the 5′-UTR of the nucleic acid molecule. In other embodiments, the TEE is located at the 3′-UTR of the nucleic acid molecule. In yet other embodiments, at least two TEE are located at the 5′-UTR and 3′-UTR of the nucleic acid molecule respectively. In one embodiment, a nucleic acid molecule of the present disclosure (e.g., mRNA) can comprise one or more copies of a TEE sequence or comprise more than one different TEE sequences. In one embodiment, different TEE sequences that are present in a nucleic acid molecule of the present disclosure can be homologues or heterologous with respect to one another.

Various TEE sequences that are known in the art and can be used in connection with the present disclosure. For example, in one embodiment, the TEE can be an internal ribosome entry site (IRES), HCV-IRES or an IRES element. Chappell et al. Proc. Natl. Acad. Sci. USA 101:9590-9594, 2004; Zhou et al. Proc. Natl. Acad. Sci. 102:6273-6278, 2005. Additional internal ribosome entry site (IRES) that can be used in connection with the present disclosure include but are not limited to those described in U.S. Pat. No. 7,468,275, U.S. Patent Publication No. 2007/0048776 and U.S. Patent Publication No. 2011/0124100 and International Patent Publication No. WO2007/025008 and International Patent Publication No. WO2001/055369, the content of each of which is enclosed herein by reference in its entirety. In one embodiment, the TEE can be those described in Supplemental Table 1 and in Supplemental Table 2 of Wellensiek et al Genome-wide profiling of human cap-independent translation-enhancing elements, Nature Methods, 2013 August; 10(8): 747-750; the content of which is incorporated by reference in its entirety.

Additional exemplary TEEs that can be used in connection with the present disclosure include but are not limited to the TEE sequences disclosed in U.S. Pat. Nos. 6,310,197, 6,849,405, 7,456,273, 7,183,395, U.S. Patent Publication No. 2009/0226470, U.S. Patent Publication No. 2013/0177581, U.S. Patent Publication No. 2007/0048776, U.S. Patent Publication No. 2011/0124100, U.S. Patent Publication No. 2009/0093049, International Patent Publication No. WO2009/075886, International Patent Publication No. WO2012/009644, and International Patent Publication No. WO1999/024595, International Patent Publication No. WO2007/025008, International Patent Publication No. WO2001/055371, European Patent No. 2610341, European Patent No. 2610340, the content of each of which is enclosed herein by reference in its entirety.

In various embodiments, a nucleic acid molecule of the present disclosure (e.g., mRNA) comprises at least one UTR that comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In one embodiment, the TEE sequences in the UTR of a nucleic acid molecule are copies of the same TEE sequence. In other embodiments, at least two TEE sequences in the UTR of a nucleic acid molecule are of different TEE sequences. In one embodiment, multiple different TEE sequences are arranged in one or more repeating patterns in the UTR region of a nucleic acid molecule. For illustrating purpose only, a repeating pattern can be, for example, ABABAB, AABBAABBAABB, ABCABCABC, or the like, where in these exemplary patterns, each capitalized letter (A, B, or C) represents a different TEE sequence. In one embodiment, at least two TEE sequences are consecutive with one another (i.e., no spacer sequence in between) in a UTR of a nucleic acid molecule. In other embodiments, at least two TEE sequences are separated by a spacer sequence. In one embodiment, a UTR can comprise a TEE sequence-spacer sequence module that is repeated at least once, at least twice, at least 3 times, at least 4 times, at least 5 times, at least 6 times, at least 7 times, at least 8 times, at least 9 times, or more than 9 times in the UTR. In any of the embodiments described in this paragraph, the UTR can be a 5′-UTR, a 3′-UTR or both 5′-UTR and 3′-UTR of a nucleic acid molecule.

In one embodiment, the UTR of a nucleic acid molecule of the present disclosure (e.g., mRNA) comprises at least one translation suppressing element that functions to decrease the amount of polypeptide or protein produced from the nucleic acid molecule. In one embodiment, the UTR of the nucleic acid molecule comprises one or more miR sequences or fragment thereof (e.g., miR seed sequences) that are recognized by one or more microRNA. In one embodiment, the UTR of the nucleic acid molecule comprises one or more stem-loop structure that downregulates translational activity of the nucleic acid molecule. Other mechanisms for suppressing translational activities associated with a nucleic acid molecules are known in the art. In any of the embodiments described in this paragraph, the UTR can be a 5′-UTR, a 3′-UTR or both 5′-UTR and 3′-UTR of a nucleic acid molecule.

### The Polyadenylation (Poly-A) Regions

During natural RNA processing, a long chain of adenosine nucleotides (poly-A region) is normally added to messenger RNA (mRNA) molecules to increase the stability of the molecule. Immediately after transcription, the 3′-end of the transcript is cleaved to free a 3′-hydroxy. Then poly-A polymerase adds a chain of adenosine nucleotides to the RNA. The process, called polyadenylation, adds a poly-A region that is between 100 and 250 residues long. Without being bound by the theory, it is contemplated that a poly-A region can confer various advantages to the nucleic acid molecule of the present disclosure.

Accordingly, in one embodiment, a nucleic acid molecule of the present disclosure (e.g., an mRNA) comprises a polyadenylation signal. In one embodiment, a nucleic acid molecule of the present disclosure (e.g., an mRNA) comprises one or more polyadenylation (poly-A) regions. In one embodiment, a poly-A region is composed entirely of adenine nucleotides or functional analogs thereof. In one embodiment, the nucleic acid molecule comprises at least one poly-A region at its 3′-end. In one embodiment, the nucleic acid molecule comprises at least one poly-A region at its 5′-end. In one embodiment, the nucleic acid molecule comprises at least one poly-A region at its 5′-end and at least one poly-A region at its 3′-end.

According to the present disclosure, the poly-A region can have varied lengths in different embodiments. Particularly, in one embodiment, the poly-A region of a nucleic acid molecule of the present disclosure is at least 30 nucleotides in length. In one embodiment, the poly-A region of a nucleic acid molecule of the present disclosure is at least 35 nucleotides in length. In one embodiment, the poly-A region of a nucleic acid molecule of the present disclosure is at least 40 nucleotides in length. In one embodiment, the poly-A region of a nucleic acid molecule of the present disclosure is at least 45 nucleotides in length. In one embodiment, the poly-A region of a nucleic acid molecule of the present disclosure is at least 50 nucleotides in length. In one embodiment, the poly-A region of a nucleic acid molecule of the present disclosure is at least 55 nucleotides in length. In one embodiment, the poly-A region of a nucleic acid molecule of the present disclosure is at least 60 nucleotides in length. In one embodiment, the poly-A region of a nucleic acid molecule of the present disclosure is at least 65 nucleotides in length. In one embodiment, the poly-A region of a nucleic acid molecule of the present disclosure is at least 70 nucleotides in length. In one embodiment, the poly-A region of a nucleic acid molecule of the present disclosure is at least 75 nucleotides in length. In one embodiment, the poly-A region of a nucleic acid molecule of the present disclosure is at least 80 nucleotides in length. In one embodiment, the poly-A region of a nucleic acid molecule of the present disclosure is at least 85 nucleotides in length. In one embodiment, the poly-A region of a nucleic acid molecule of the present disclosure is at least 90 nucleotides in length. In one embodiment, the poly-A region of a nucleic acid molecule of the present disclosure is at least 95 nucleotides in length. In one embodiment, the poly-A region of a nucleic acid molecule of the present disclosure is at least 100 nucleotides in length. In one embodiment, the poly-A region of a nucleic acid molecule of the present disclosure is at least 110 nucleotides in length. In one embodiment, the poly-A region of a nucleic acid molecule of the present disclosure is at least 120 nucleotides in length. In one embodiment, the poly-A region of a nucleic acid molecule of the present disclosure is at least 130 nucleotides in length. In one embodiment, the poly-A region of a nucleic acid molecule of the present disclosure is at least 140 nucleotides in length. In one embodiment, the poly-A region of a nucleic acid molecule of the present disclosure is at least 150 nucleotides in length. In one embodiment, the poly-A region of a nucleic acid molecule of the present disclosure is at least 160 nucleotides in length. In one embodiment, the poly-A region of a nucleic acid molecule of the present disclosure is at least 170 nucleotides in length. In one embodiment, the poly-A region of a nucleic acid molecule of the present disclosure is at least 180 nucleotides in length. In one embodiment, the poly-A region of a nucleic acid molecule of the present disclosure is at least 190 nucleotides in length. In one embodiment, the poly-A region of a nucleic acid molecule of the present disclosure is at least 200 nucleotides in length. In one embodiment, the poly-A region of a nucleic acid molecule of the present disclosure is at least 225 nucleotides in length. In one embodiment, the poly-A region of a nucleic acid molecule of the present disclosure is at least 250 nucleotides in length. In one embodiment, the poly-A region of a nucleic acid molecule of the present disclosure is at least 275 nucleotides in length. In one embodiment, the poly-A region of a nucleic acid molecule of the present disclosure is at least 300 nucleotides in length. In one embodiment, the poly-A region of a nucleic acid molecule of the present disclosure is at least 350 nucleotides in length. In one embodiment, the poly-A region of a nucleic acid molecule of the present disclosure is at least 400 nucleotides in length. In one embodiment, the poly-A region of a nucleic acid molecule of the present disclosure is at least 450 nucleotides in length. In one embodiment, the poly-A region of a nucleic acid molecule of the present disclosure is at least 500 nucleotides in length. In one embodiment, the poly-A region of a nucleic acid molecule of the present disclosure is at least 600 nucleotides in length. In one embodiment, the poly-A region of a nucleic acid molecule of the present disclosure is at least 700 nucleotides in length. In one embodiment, the poly-A region of a nucleic acid molecule of the present disclosure is at least 800 nucleotides in length. In one embodiment, the poly-A region of a nucleic acid molecule of the present disclosure is at least 900 nucleotides in length. In one embodiment, the poly-A region of a nucleic acid molecule of the present disclosure is at least 1000 nucleotides in length. In one embodiment, the poly-A region of a nucleic acid molecule of the present disclosure is at least 1100 nucleotides in length. In one embodiment, the poly-A region of a nucleic acid molecule of the present disclosure is at least 1200 nucleotides in length. In one embodiment, the poly-A region of a nucleic acid molecule of the present disclosure is at least 1300 nucleotides in length. In one embodiment, the poly-A region of a nucleic acid molecule of the present disclosure is at least 1400 nucleotides in length. In one embodiment, the poly-A region of a nucleic acid molecule of the present disclosure is at least 1500 nucleotides in length. In one embodiment, the poly-A region of a nucleic acid molecule of the present disclosure is at least 1600 nucleotides in length. In one embodiment, the poly-A region of a nucleic acid molecule of the present disclosure is at least 1700 nucleotides in length. In one embodiment, the poly-A region of a nucleic acid molecule of the present disclosure is at least 1800 nucleotides in length. In one embodiment, the poly-A region of a nucleic acid molecule of the present disclosure is at least 1900 nucleotides in length. In one embodiment, the poly-A region of a nucleic acid molecule of the present disclosure is at least 2000 nucleotides in length. In one embodiment, the poly-A region of a nucleic acid molecule of the present disclosure is at least 2250 nucleotides in length. In one embodiment, the poly-A region of a nucleic acid molecule of the present disclosure is at least 2500 nucleotides in length. In one embodiment, the poly-A region of a nucleic acid molecule of the present disclosure is at least 2750 nucleotides in length. In one embodiment, the poly-A region of a nucleic acid molecule of the present disclosure is at least 3000 nucleotides in length.

In one embodiment, length of a poly-A region in a nucleic acid molecule can be selected based on the overall length of the nucleic acid molecule, or a portion thereof (such as the length of the coding region or the length of an open reading frame of the nucleic acid molecule, etc.). For example, in one embodiment, the poly-A region accounts for about 5%, 10%, 15%, 20%, 25%, 30%, 35%, 40%, 45%, 50%, 55%, 60%, 65%, 70%, 75%, 80%, 85%, 90%, 95% or more of the total length of nucleic acid molecule containing the poly-A region.

Without being bound by the theory, it is contemplated that certain RNA-binding proteins can bind to the poly-A region located at the 3′-end of an mRNA molecule. These poly-A binding proteins (PABP) can modulate mRNA expression, such as interacting with translation initiation machinery in a cell and/or protecting the 3′-poly-A tails from degradation. Accordingly, in one embodiment, the nucleic acid molecule of the present disclosure (e.g., mRNA) comprises at least one binding site for poly-A binding protein (PABP). In other embodiments, the nucleic acid molecule is conjugated or complex with a PABP before loaded into a delivery vehicle (e.g., lipid nanoparticles).

In one embodiment, the nucleic acid molecule of the present disclosure (e.g., mRNA) comprises a poly-A-G Quartet. The G-quartet is a cyclic hydrogen bonded array of four guanosine nucleotides that can be formed by G-rich sequences in both DNA and RNA. In this embodiment, the G-quartet is incorporated at the end of the poly-A region. The resultant polynucleotides (e.g., mRNA) may be assayed for stability, protein production and other parameters including half-life at various time points. It has been discovered that the polyA-G quartet structure results in protein production equivalent to at least 75% of that seen using a poly-A region of 120 nucleotides alone.

In one embodiment, the nucleic acid molecule of the present disclosure (e.g., mRNA) may include a poly-A region and may be stabilized by the addition of a 3′-stabilizing region. In one embodiment, the 3′-stabilizing region which may be used to stabilize a nucleic acid molecule (e.g., mRNA) including the poly-A or poly-A-G Quartet structures as described in International Patent Publication No. WO2013/103659, the content of which is incorporated herein by reference in its entirety.

In other embodiments, the 3′-stabilizing region which may be used in connection with the nucleic acid molecules of the present disclosure include a chain termination nucleoside such as but is not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein.

### Secondary Structure

Without being bound by the theory, it is contemplated that a stem-loop structure can direct RNA folding, protect structural stability of a nucleic acid molecule (e.g., mRNA), provide recognition sites for RNA binding proteins, and serve as a substrate for enzymatic reactions. For example, the incorporation of a miR sequence and/or a TEE sequence changes the shape of the stem loop region which may increase and/or decrease translation (Kedde et al. A Pumilio-induced RNA structure switch in p27-3′UTR controls miR-221 and miR-222 accessibility. Nat Cell Biol., 2010 October; 12(10):1014-20, the content of which is herein incorporated by reference in its entirety).

Accordingly, in one embodiment, the nucleic acid molecules as described herein (e.g., mRNA) or a portion thereof may assume a stem-loop structure, such as but is not limited to a histone stem loop. In one embodiment, the stem-loop structure is formed from a stem-loop sequence that is about 25 or about 26 nucleotides in length such as, but not limited to, those as described in International Patent Publication No. WO2013/103659, the content of which is incorporated herein by reference in its entirety. Additional examples of stem-loop sequences include those described in International Patent Publication No. WO2012/019780 and International Patent Publication No. WO201502667, the contents of which are incorporated herein by reference. In one embodiment, the step-loop sequence comprises a TEE as described herein. In one embodiment, the step-loop sequence comprises a miR sequence as described herein. In specific embodiments, the stem loop sequence may include a miR-122 seed sequence. In specific embodiments, the nucleic acid molecule comprises the two stem-loop sequence described in International Patent Publication No. WO2021204175, the entirety of which is incorporated herein by reference.

In one embodiment, the nucleic acid molecule of the present disclosure (e.g., mRNA) comprises a stem-loop sequence located upstream (to the 5′-end) of the coding region in a nucleic acid molecule. In one embodiment, the stem-loop sequence is located within the 5′-UTR of the nucleic acid molecule. In one embodiment, the nucleic acid molecule of the present disclosure (e.g., mRNA) comprises a stem-loop sequence located downstream (to the 3′-end) of the coding region in a nucleic acid molecule. In one embodiment, the stem-loop sequence is located within the 3′-UTR of the nucleic acid molecule. In some cases, a nucleic acid molecule can contain more than one stem-loop sequences. In some embodiment, the nucleic acid molecule comprises at least one stem-loop sequence in the 5′-UTR, and at least one stem-loop sequence in the 3′-UTR.

In one embodiment, a nucleic acid molecule comprising a stem-loop structure further comprises a stabilization region. In some embodiment, the stabilization region comprises at least one chain terminating nucleoside that functions to slow down degradation and thus increases the half-life of the nucleic acid molecule. Exemplary chain terminating nucleoside that can be used in connection with the present disclosure include but are not limited to 3′-deoxyadenosine (cordycepin), 3′-deoxyuridine, 3′-deoxycytosine, 3′-deoxyguanosine, 3′-deoxythymine, 2′,3′-dideoxynucleosides, such as 2′,3′-dideoxyadenosine, 2′,3′-dideoxyuridine, 2′,3′-dideoxycytosine, 2′,3′-dideoxyguanosine, 2′,3′-dideoxythymine, a 2′-deoxynucleoside, or an O-methylnucleoside, 3′-deoxynucleoside, 2′,3′-dideoxynucleoside 3′-O-methylnucleosides, 3′-O-ethylnucleosides, 3′-arabinosides, and other alternative nucleosides known in the art and/or described herein. In other embodiments, a stem-loop structure may be stabilized by an alteration to the 3′-region of the polynucleotide that can prevent and/or inhibit the addition of oligio(U) (International Patent Publication No. WO2013/103659, incorporated herein by reference in its entirety).

In one embodiment, a nucleic acid molecule of the present disclosure comprises at least one stem-loop sequence and a poly-A region or polyadenylation signal. Non-limiting examples of polynucleotide sequences comprising at least one stem-loop sequence and a poly-A region or a polyadenylation signal include those described in International Patent Publication No. WO2013/120497, International Patent Publication No. WO2013/120629, International Patent Publication No. WO2013/120500, International Patent Publication No. WO2013/120627, International Patent Publication No. WO2013/120498, International Patent Publication No. WO2013/120626, International Patent Publication No. WO2013/120499 and International Patent Publication No. WO2013/120628, the content of each of which is incorporated herein by reference in its entirety.

In one embodiment, the nucleic acid molecule comprising a stem-loop sequence and a poly-A region or a polyadenylation signal can encode for a pathogen antigen or fragment thereof such as the polynucleotide sequences described in International Patent Publication No. WO2013/120499 and International Patent Publication No. WO2013/120628, the content of each of which is incorporated herein by reference in its entirety.

In one embodiment, the nucleic acid molecule comprising a stem-loop sequence and a poly-A region or a polyadenylation signal can encode for a therapeutic protein such as the polynucleotide sequences described in International Patent Publication No. WO2013/120497 and International Patent Publication No. WO2013/120629, the content of each of which is incorporated herein by reference in its entirety.

In one embodiment, the nucleic acid molecule comprising a stem-loop sequence and a poly-A region or a polyadenylation signal can encode for a tumor antigen or fragment thereof such as the polynucleotide sequences described in International Patent Publication No. WO2013/120500 and International Patent Publication No. WO2013/120627, the content of each of which is incorporated herein by reference in its entirety.

In one embodiment, the nucleic acid molecule comprising a stem-loop sequence and a poly-A region or a polyadenylation signal can code for an allergenic antigen or an autoimmune self-antigen such as the polynucleotide sequences described in International Patent Publication No. WO2013/120498 and International Patent Publication No. WO2013/120626, the content of each of which is incorporated herein by reference in its entirety.

### Functional Nucleotide Analogs

In one embodiment, a payload nucleic acid molecule described herein contains only canonical nucleotides selected from A (adenosine), G (guanosine), C (cytosine), U (uridine), and T (thymidine). Without being bound by the theory, it is contemplated that certain functional nucleotide analogs can confer useful properties to a nucleic acid molecule. Examples of such as useful properties in the context of the present disclosure include but are not limited to increased stability of the nucleic acid molecule, reduced immunogenicity of the nucleic acid molecule in inducing innate immune responses, enhanced production of protein encoded by the nucleic acid molecule, increased intracellular delivery and/or retention of the nucleic acid molecule, and/or reduced cellular toxicity of the nucleic acid molecule, etc.

Accordingly, in one embodiment, a payload nucleic acid molecule comprises at least one functional nucleotide analog as described herein. In one embodiment, the functional nucleotide analog contains at least one chemical modification to the nucleobase, the sugar group and/or the phosphate group. Accordingly, a payload nucleic acid molecule comprising at least one functional nucleotide analog contains at least one chemical modification to the nucleobases, the sugar groups, and/or the internucleoside linkage. Exemplary chemical modifications to the nucleobases, sugar groups, or internucleoside linkages of a nucleic acid molecule are provided herein.

As described herein, ranging from 0% to 100% of all nucleotides in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of all nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In one embodiment, a single nucleic acid molecule can contain different sugar modifications, different nucleobase modifications, and/or different types internucleoside linkages (e.g., backbone structures).

As described herein, ranging from 0% to 100% of all nucleotides of a kind (e.g., all purine-containing nucleotides as a kind, or all pyrimidine-containing nucleotides as a kind, or all A, G, C, T or U as a kind) in a payload nucleic acid molecule can be functional nucleotide analogs as described herein. For example, in various embodiments, from about 1% to about 20%, from about 1% to about 25%, from about 1% to about 50%, from about 1% to about 60%, from about 1% to about 70%, from about 1% to about 80%, from about 1% to about 90%, from about 1% to about 95%, from about 10% to about 20%, from about 10% to about 25%, from about 10% to about 50%, from about 10% to about 60%, from about 10% to about 70%, from about 10% to about 80%, from about 10% to about 90%, from about 10% to about 95%, from about 10% to about 100%, from about 20% to about 25%, from about 20% to about 50%, from about 20% to about 60%, from about 20% to about 70%, from about 20% to about 80%, from about 20% to about 90%, from about 20% to about 95%, from about 20% to about 100%, from about 50% to about 60%, from about 50% to about 70%, from about 50% to about 80%, from about 50% to about 90%, from about 50% to about 95%, from about 50% to about 100%, from about 70% to about 80%, from about 70% to about 90%, from about 70% to about 95%, from about 70% to about 100%, from about 80% to about 90%, from about 80% to about 95%, from about 80% to about 100%, from about 90% to about 95%, from about 90% to about 100%, or from about 95% to about 100% of a kind of nucleotides in a nucleic acid molecule are functional nucleotide analogs described herein. In any of these embodiments, a functional nucleotide analog can be present at any position(s) of a nucleic acid molecule, including the 5′-terminus, 3′-terminus, and/or one or more internal positions. In one embodiment, a single nucleic acid molecule can contain different sugar modifications, different nucleobase modifications, and/or different types internucleoside linkages (e.g., backbone structures).

### Modification to Nucleobases

In one embodiment, a functional nucleotide analog contains a non-canonical nucleobase. In one embodiment, canonical nucleobases (e.g., adenine, guanine, uracil, thymine, and cytosine) in a nucleotide can be modified or replaced to provide one or more functional analogs of the nucleotide. Exemplary modification to nucleobases include but are not limited to one or more substitutions or modifications including but not limited to alkyl, aryl, halo, oxo, hydroxyl, alkyloxy, and/or thio substitutions; one or more fused or open rings, oxidation, and/or reduction.

In one embodiment, the non-canonical nucleobase is a modified uracil. Exemplary nucleobases and nucleosides having an modified uracil include pseudouridine (Y7), pyridin-4-one ribonucleoside, 5-aza-uracil, 6-aza-uracil, 2-thio-5-aza-uracil, 2-thio-uracil (s²U), 4-thio-uracil (s⁴U), 4-thio-pseudouridine, 2-thio-pseudouridine, 5-hydroxy-uracil (ho⁵U), 5-aminoallyl-uracil, 5-halo-uracil (e.g., 5-iodo-uracil or 5-bromo-uracil), 3-methyl-uracil (m³U), 5-methoxy-uracil (mo⁵U), uracil 5-oxyacetic acid (cmo⁵U), uracil 5-oxyacetic acid methyl ester (mcmo⁵U), 5-carboxymethyl-uracil (cm⁵U), 1-carboxymethyl-pseudouridine, 5-carboxyhydroxymethyl-uracil (chm⁵U), 5-carboxyhydroxymethyl-uracil methyl ester (mchm⁵U), 5-methoxycarbonylmethyl-uracil (mcm⁵U), 5-methoxycarbonylmethyl-2-thio-uracil (mcm⁵s²U), 5-aminomethyl-2-thio-uracil (nm⁵s²U), 5-methylaminomethyl-uracil (mnm⁵U), 5-methylaminomethyl-2-thio-uracil (mnm⁵s²U), 5-methylaminomethyl-2-seleno-uracil (mnm⁵se²U), 5-carbamoylmethyl-uracil (ncm⁵U), 5-carboxymethylaminomethyl-uracil (cmnm⁵U), 5-carboxymethylaminomethyl-2-thio-uracil (cmnm⁵s²U), 5-propynyl-uracil, 1-propynyl-pseudouracil, 5-taurinomethyl-uracil (Tm⁵U), 1-taurinomethyl-pseudouridine, 5-taurinomethyl-2-thio-uracil(τm⁵5s²U), 1-taurinomethyl-4-thio-pseudouridine, 5-methyl-uracil (m⁵U, i.e., having the nucleobase deoxythymine), 1-methyl-pseudouridine (m¹ψ), 1-ethyl-pseudouridine (Et¹ψ), 5-methyl-2-thio-uracil (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouracil (D), dihydropseudouridine, 5,6-dihydrouracil, 5-methyl-dihydrouracil (m⁵D), 2-thio-dihydrouracil, 2-thio-dihydropseudouridine, 2-methoxy-uracil, 2-methoxy-4-thio-uracil, 4-methoxy-pseudouridine, 4-methoxy-2-thio-pseudouridine, N1-methyl-pseudouridine, 3-(3-amino-3-carboxypropyl)uracil (acp³U), 1-methyl-3-(3-amino-3-carboxypropyl)pseudouridine (acp³ψ), 5-(isopentenylaminomethyl)uracil (m⁵U), 5-(isopentenylaminomethyl)-2-thio-uracil (M⁵s²U), 5,2′-O-dimethyl-uridine (m⁵Um), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uracil, deoxythymidine, 5-(2-carbomethoxyvinyl)-uracil, 5-(carbamoylhydroxymethyl)-uracil, 5-carbamoylmethyl-2-thio-uracil, 5-carboxymethyl-2-thio-uracil, 5-cyanomethyl-uracil, 5-methoxy-2-thio-uracil, and 5-[3-(1-E-propenylamino)]uracil.

In one embodiment, the non-canonical nucleobase is a modified cytosine. Exemplary nucleobases and nucleosides having a modified cytosine include 5-aza-cytosine, 6-aza-cytosine, pseudoisocytidine, 3-methyl-cytosine (m3C), N4-acetyl-cytosine (ac4C), 5-formyl-cytosine (f5C), N4-methyl-cytosine (m4C), 5-methyl-cytosine (m5C), 5-halo-cytosine (e.g., 5-iodo-cytosine), 5-hydroxymethyl-cytosine (hm5C), 1-methyl-pseudoisocytidine, pyrrolo-cytosine, pyrrolo-pseudoisocytidine, 2-thio-cytosine (s2C), 2-thio-5-methyl-cytosine, 4-thio-pseudoisocytidine, 4-thio-1-methyl-pseudoisocytidine, 4-thio-i-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytosine, 2-methoxy-5-methyl-cytosine, 4-methoxy-pseudoisocytidine, 4-methoxy-i-methyl-pseudoisocytidine, lysidine (k2C), 5,2′-O-dimethyl-cytidine (m5Cm), N4-acetyl-2′-O-methyl-cytidine (ac4Cm), N4,2′-O-dimethyl-cytidine (m4Cm), 5-formyl-2′-O-methyl-cytidine (fSCm), N4,N4,2′-O-trimethyl-cytidine (m42Cm), 1-thio-cytosine, 5-hydroxy-cytosine, 5-(3-azidopropyl)-cytosine, and 5-(2-azidoethyl)-cytosine.

In one embodiment, the non-canonical nucleobase is a modified adenine. Exemplary nucleobases and nucleosides having an alternative adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenine (m1A), 2-methyl-adenine (m2A), N6-methyl-adenine (m6A), 2-methylthio-N6-methyl-adenine (ms2m6A), N6-isopentenyl-adenine (i6A), 2-methylthio-N6-isopentenyl-adenine (ms2i6A), N6-(cis-hydroxyisopentenyl)adenine (io6A), 2-methylthio-N6-(cis-hydroxyisopentenyl)adenine (ms2io6A), N6-glycinylcarbamoyl-adenine (g6A), N6-threonylcarbamoyl-adenine (t6A), N6-methyl-N6-threonylcarbamoyl-adenine (m6t6A), 2-methylthio-N6-threonylcarbamoyl-adenine (ms2g6A), N6,N6-dimethyl-adenine (m62A), N6-hydroxynorvalylcarbamoyl-adenine (hn6A), 2-methylthio-N6-hydroxynorvalylcarbamoyl-adenine (ms2hn6A), N6-acetyl-adenine (ac6A), 7-methyl-adenine, 2-methylthio-adenine, 2-methoxy-adenine, N6,2′-0-dimethyl-adenosine (m6Am), N6,N6,2′-O-trimethyl-adenosine (m62Am), 1,2′-O-dimethyl-adenosine (m1Am), 2-amino-N6-methyl-purine, 1-thio-adenine, 8-azido-adenine, N6-(19-amino-pentaoxanonadecyl)-adenine, 2,8-dimethyl-adenine, N6-formyl-adenine, and N6-hydroxymethyl-adenine.

In one embodiment, the non-canonical nucleobase is a modified guanine. Exemplary nucleobases and nucleosides having a modified guanine include inosine (I), 1-methyl-inosine (m1I), wyosine (imG), methylwyosine (mimG), 4-demethyl-wyosine (imG-14), isowyosine (imG2), wybutosine (yW), peroxywybutosine (o2yW), hydroxywybutosine (OHyW), undermodified hydroxywybutosine (OHyW*), 7-deaza-guanine, queuosine (Q), epoxyqueuosine (oQ), galactosyl-queuosine (galQ), mannosyl-queuosine (manQ), 7-cyano-7-deaza-guanine (preQO), 7-aminomethyl-7-deaza-guanine (preQ1), archaeosine (G+), 7-deaza-8-aza-guanine, 6-thio-guanine, 6-thio-7-deaza-guanine, 6-thio-7-deaza-8-aza-guanine, 7-methyl-guanine (m7G), 6-thio-7-methyl-guanine, 7-methyl-inosine, 6-methoxy-guanine, 1-methyl-guanine (m1G), N2-methyl-guanine (m2G), N2,N2-dimethyl-guanine (m22G), N2,7-dimethyl-guanine (m2,7G), N2, N2,7-dimethyl-guanine (m2,2,7G), 8-oxo-guanine, 7-methyl-8-oxo-guanine, 1-methyl-6-thio-guanine, N2-methyl-6-thio-guanine, N2,N2-dimethyl-6-thio-guanine, N2-methyl-2′-O-methyl-guanosine (m2Gm), N2,N2-dimethyl-2′-O-methyl-guanosine (m22Gm), 1-methyl-2′-O-methyl-guanosine (m1Gm), N2,7-dimethyl-2′-O-methyl-guanosine (m2,7Gm), 2′-O-methyl-inosine (Im), 1,2′-O-dimethyl-inosine (m1Im), 1-thio-guanine, and O-6-methyl-guanine.

In one embodiment, the non-canonical nucleobase of a functional nucleotide analog can be independently a purine, a pyrimidine, a purine or pyrimidine analog. For example, in one embodiment, the non-canonical nucleobase can be modified adenine, cytosine, guanine, uracil, or hypoxanthine. In other embodiments, the non-canonical nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxy and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; or 1,3,5 triazine.

### Modification to the Sugar

In one embodiment, a functional nucleotide analog contains a non-canonical sugar group. In various embodiments, the non-canonical sugar group can be a 5-carbon or 6-carbon sugar (such as pentose, ribose, arabinose, xylose, glucose, galactose, or a deoxy derivative thereof) with one or more substitutions, such as a halo group, a hydroxy group, a thiol group, an alkyl group, an alkoxy group, an alkenyloxy group, an alkynyloxy group, an cycloalkyl group, an aminoalkoxy group, an alkoxyalkoxy group, an hydroxyalkoxy group, an amino group, an azido group, an aryl group, an aminoalkyl group, an aminoalkenyl group, an aminoalkynyl group, etc.

Generally, RNA molecules contains the ribose sugar group, which is a 5-membered ring having an oxygen. Exemplary, non-limiting alternative nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino (that also has a phosphoramidate backbone)); multicyclic forms (e.g., tricyclo and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone).

In one embodiment, the sugar group contains one or more carbons that possess the opposite stereochemical configuration of the corresponding carbon in ribose. Thus, a nucleic acid molecule can include nucleotides containing, e.g., arabinose or L-ribose, as the sugar. In one embodiment, the nucleic acid molecule includes at least one nucleoside wherein the sugar is L-ribose, 2′-O-methyl-ribose, 2′-fluoro-ribose, arabinose, hexitol, an LNA, or a PNA.

### Modifications to the Internucleoside Linkage

In one embodiment, the payload nucleic acid molecule of the present disclosure can contain one or more modified internucleoside linkage (e.g., phosphate backbone). Backbone phosphate groups can be altered by replacing one or more of the oxygen atoms with a different substituent.

In one embodiment, the functional nucleotide analogs can include the replacement of an unaltered phosphate moiety with another internucleoside linkage as described herein. Examples of alternative phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be altered by the replacement of a linking oxygen with nitrogen (bridged phosphoramidates), sulfur (bridged phosphorothioates), and carbon (bridged methylene-phosphonates).

The alternative nucleosides and nucleotides can include the replacement of one or more of the non-bridging oxygens with a borane moiety (BH₃), sulfur (thio), methyl, ethyl, and/or methoxy. As a non-limiting example, two non-bridging oxygens at the same position (e.g., the alpha (α), beta (β) or gamma (γ) position) can be replaced with a sulfur (thio) and a methoxy. The replacement of one or more of the oxygen atoms at the position of the phosphate moiety (e.g., α-thio phosphate) is provided to confer stability (such as against exonucleases and endonucleases) to RNA and DNA through the unnatural phosphorothioate backbone linkages. Phosphorothioate DNA and RNA have increased nuclease resistance and subsequently a longer half-life in a cellular environment.

Other internucleoside linkages that may be employed according to the present disclosure, including internucleoside linkages which do not contain a phosphorous atom, are described herein.

Additional examples of nucleic acid molecules (e.g., mRNA), compositions, formulations and/or methods associated therewith that can be used in connection with the present disclosure further include those described in WO2002/098443, WO2003/051401, WO2008/052770, WO2009127230, WO2006122828, WO2008/083949, WO2010088927, WO2010/037539, WO2004/004743, WO2005/016376, WO2006/024518, WO2007/095976, WO2008/014979, WO2008/077592, WO2009/030481, WO2009/095226, WO2011069586, WO2011026641, WO2011/144358, WO2012019780, WO2012013326, WO2012089338, WO2012113513, WO2012116811, WO2012116810, WO2013113502, WO2013113501, WO2013113736, WO2013143698, WO2013143699, WO2013143700, WO2013/120626, WO2013120627, WO2013120628, WO2013120629, WO2013174409, WO2014127917, WO2015/024669, WO2015/024668, WO2015/024667, WO2015/024665, WO2015/024666, WO2015/024664, WO2015101415, WO2015101414, WO2015024667, WO2015062738, WO2015101416, the content of each of which is incorporated herein in its entirety.

### 6.3.2 Formulation

The lipid nanoparticle compositions described herein can include at least one lipid component and one or more additional components, such as a therapeutic and/or prophylactic agent (e.g., the therapeutic nucleic acid described herein). A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

In one embodiment, the therapeutic and/or prophylactic agent encapsulated in the nanoparticles can be delivered to a host cell in vitro, for example, by contacting the host cell with the nanoparticle composition, or in vivo, for example, by administering the nanoparticle composition to a subject containing the host cell. In one embodiment, upon delivery, the therapeutic nucleic acid molecule encapsulated in the nanoparticle can be expressed via the host cell endogenous transcription and translation machinery.

In one embodiment, the nanoparticle composition comprises a non-lipid component comprising a therapeutic agent. In one embodiment, the therapeutic agent is a nucleic acid molecule. In one embodiment, the therapeutic agent is an mRNA molecule.

In one embodiment, upon delivery of the nucleic acid containing nanoparticle composition to a host cell, the nucleic acid is expressed to form RNA and/or protein via the host cell endogenous transcription and/or translation machinery. In one embodiment, the expression level of the nucleic acid formulated in LNP is enhanced as compared to the nucleic acid formulated in a reference LNP composition.

Accordingly, the nanoparticle composition described herein can be used in a method of expressing an mRNA in a host cell or tissue of a host subject, wherein the method comprises formulating the mRNA within a nanoparticle composition and delivering the nanoparticle composition to the host cells or the host subject; and wherein the delivered mRNA is expressed in the host cell or in the host subject. In one embodiment, the host cell is a mammalian cell (such as a cell originated from human or a non-human vertebrate). In one embodiment, the host subject is a mammal (such as human or non-human vertebrate). In one embodiment, delivering the nanoparticle composition can be performed by contacting the nanoparticle composition in vitro with the host cells. In one embodiment, delivering the nanoparticle composition can be performed by administering the nanoparticle composition in vivo to the host subject.

In some embodiment, the therapeutic agent to lipid ratio in the lipid nanoparticle composition (i.e., N/P, were N represents the moles of cationic lipid and P represents the moles of phosphate present as part of the nucleic acid backbone) range from 9:1 to 20:1, for example 10:1 to 15:1. Exemplary N/P ranges include about 9:1. about 10:1, about 11:1, about 12:1, about 13:1, about 14:1, about 15:1, about 16:1, about 17:1, about 18:1, about 19:1 and about 20:1.

In one embodiment, provided herein is a lipid nanoparticle for use in targeted delivery of therapeutical payloads. Nanoparticle compositions can be designed for one or more specific applications or targets. For example, a nanoparticle composition can be designed to deliver a therapeutic and/or prophylactic agent such as an RNA to a particular cell, tissue, organ, or system or group thereof in a mammal's body. Physiochemical properties of nanoparticle compositions can be altered in order to increase selectivity for particular bodily targets. For instance, particle sizes can be adjusted based on the fenestration sizes of different organs. The therapeutic and/or prophylactic agent included in a nanoparticle composition can also be selected based on the desired delivery target or targets. For example, a therapeutic and/or prophylactic agent can be selected for a particular indication, condition, disease, or disorder and/or for delivery to a particular cell, tissue, organ, or system or group thereof (e.g., localized or specific delivery). In certain embodiments, a nanoparticle composition can include an mRNA encoding a polypeptide of interest capable of being translated within a cell to produce the polypeptide of interest. Such a composition can be designed to be specifically delivered to a particular organ. In one embodiment, a composition can be designed to be specifically delivered to a mammalian liver. In one embodiment, a composition can be designed to be specifically delivered to a mammalian lung. In one embodiment, a composition can be designed to be specifically delivered to a site of interest, such as a site of inflammation, a site of cancer or a site of infection.

The amount of a therapeutic and/or prophylactic agent in a nanoparticle composition can depend on the size, composition, desired target and/or application, or other properties of the nanoparticle composition as well as on the properties of the therapeutic and/or prophylactic agent. For example, the amount of an RNA useful in a nanoparticle composition can depend on the size, sequence, and other characteristics of the RNA. The relative amounts of a therapeutic and/or prophylactic agent and other elements (e.g., lipids) in a nanoparticle composition can also vary. In one embodiment, the wt/wt ratio of the lipid component to a therapeutic and/or prophylactic agent in a nanoparticle composition can be from about 5:1 to about 60:1, such as about 5:1, 6:1, 7:1, 8:1, 9:1, 10:1, 11:1, 12:1, 13:1, 14:1, 15:1, 16:1, 17:1, 18:1, 19:1,20:1,22:1,25:1,30:1,35:1,40:1,45:1,50:1, and 60:1. For example, the wt/wt ratio of the lipid component to a therapeutic and/or prophylactic agent can be from about 10:1 to about 40:1. In certain embodiments, the wt/wt ratio is about 20:1. The amount of a therapeutic and/or prophylactic agent in a nanoparticle composition can, for example, be measured using absorption spectroscopy (e.g., ultraviolet-visible spectroscopy).

In one embodiment, a nanoparticle composition includes one or more RNAs, and the one or more RNAs, lipids, and amounts thereof can be selected to provide a specific N:P ratio.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

In various embodiments, the mean size of a nanoparticle composition can be between 10s of nm and 100s of nm. For example, the mean size can be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In one embodiment, the lipid nanoparticle composition comprises a plurality of nanoparticles, and the mean size of the plurality of nanoparticles is from about 40 nm to about 150 nm. In one embodiment, the mean size of the plurality of particles is from about 50 nm to about 100 nm. In one embodiment, the mean size of the plurality of particles is about 95 nm.

In one embodiment, the mean size of a three-lipid-component lipid nanoparticle composition provided herein (with no phospholipid component, or reduced/insignificant amount of phospholipid component) has an increased size, as compared to the mean size of the conventional four-lipid-component lipid nanoparticle composition, such as a four-lipid-component lipid nanoparticle wherein the molar ratio of the cationic lipid:the steroid:the phospholipid:the polymer conjugated lipid is about 50:38.5:10:1.5. In one embodiment, the mean size of a three-lipid-component nanoparticle composition provided herein has an increase in size of at least about 30%, at least about 40%, at least about 50%, at least about 60%, at least about 70%, or at least about 80%. In one embodiment, the mean size of a three-lipid-component nanoparticle composition provided herein has an increase in size of no more than about 90%, no more than about 100%, or no more than about 110%. In one embodiment, the mean size of a three-lipid-component nanoparticle composition provided herein is from about 90 nm to about 150 nm. In one embodiment, the mean size (of a three-lipid-component nanoparticle composition provided herein) is from about 100 nm to about 140 nm. In one embodiment, the mean size is from about 110 nm to about 130 nm. In one embodiment, the mean size is from about 100 nm to about 110 nm. In one embodiment, the mean size is from about 110 nm to about 120 nm. In one embodiment, the mean size is from about 120 nm to about 130 nm. In one embodiment, the mean size is about 90 nm. In one embodiment, the mean size is about 95 nm. In one embodiment, the mean size is about 100 nm. In one embodiment, the mean size is about 105 nm. In one embodiment, the mean size is about 110 nm. In one embodiment, the mean size is about 115 nm. In one embodiment, the mean size is about 120 nm. In one embodiment, the mean size is about 125 nm. In one embodiment, the mean size is about 130 nm. In one embodiment, the mean size is about 135 nm. In one embodiment, the mean size is about 140 nm. In one embodiment, the mean size is about 145 nm. In one embodiment, the mean size is about 150 nm. In one embodiment, the mean size is about 155 nm.

A nanoparticle composition can be relatively homogenous. A polydispersity index can be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In one embodiment, the polydispersity index of a nanoparticle composition can be from about 0.10 to about 0.20. In one embodiment, the lipid nanoparticle composition comprises a plurality of nanoparticles, and the polydispersity index (PDI) of the nanoparticle composition is from about 0 to about 0.25. In one embodiment, the PDI of the nanoparticle composition is less than 0.1.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In one embodiment, the zeta potential of a nanoparticle composition can be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic agent describes the amount of therapeutic and/or prophylactic agent that is encapsulated or otherwise associated with a nanoparticle composition after preparation, relative to the initial amount provided. The encapsulation efficiency is desirably high (e.g., close to 100%). The encapsulation efficiency can be measured, for example, by comparing the amount of therapeutic and/or prophylactic agent in a solution containing the nanoparticle composition before and after breaking up the nanoparticle composition with one or more organic solvents or detergents. Fluorescence can be used to measure the amount of free therapeutic and/or prophylactic agent (e.g., RNA) in a solution. For the nanoparticle compositions described herein, the encapsulation efficiency of a therapeutic and/or prophylactic agent can be at least 50%, for example 50%, 55%, 60%, 65%, 70%, 75%, 80%, 85%, 90%, 91%, 92%, 93%, 94%, 95%, 96%, 97%, 98%, 99%, or 100%.

A nanoparticle composition can optionally comprise one or more coatings. For example, a nanoparticle composition can be formulated in a capsule, film, or tablet having a coating. A capsule, film, or tablet including a composition described herein can have any useful size, tensile strength, hardness, or density.

### 6.3.3 Pharmaceutical Compositions

According to the present disclosure, nanoparticle compositions can be formulated in whole or in part as pharmaceutical compositions. Pharmaceutical compositions can include one or more nanoparticle compositions. For example, a pharmaceutical composition can include one or more nanoparticle compositions including one or more different therapeutic and/or prophylactic agents. Pharmaceutical compositions can further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients can be used in any pharmaceutical composition, except insofar as any conventional excipient or accessory ingredient can be incompatible with one or more components of a nanoparticle composition. An excipient or accessory ingredient can be incompatible with a component of a nanoparticle composition if its combination with the component can result in any undesirable biological effect or otherwise deleterious effect.

In one embodiment, one or more excipients or accessory ingredients can make up greater than 50% of the total mass or volume of a pharmaceutical composition including a nanoparticle composition. For example, the one or more excipients or accessory ingredients can make up 50%, 60%, 70%, 80%, 90%, or more of a pharmaceutical convention. In one embodiment, a pharmaceutically acceptable excipient is at least 95%, at least 96%, at least 97%, at least 98%, at least 99%, or 100% pure. In one embodiment, an excipient is approved for use in humans and for veterinary use. In one embodiment, an excipient is approved by United States Food and Drug Administration. In one embodiment, an excipient is pharmaceutical grade. In one embodiment, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition can comprise between 0.1% and 100% (wt/wt) of one or more nanoparticle compositions.

In certain embodiments, the nanoparticle compositions and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). In certain embodiments, the disclosure also relates to a method of maintaining or increasing stability of the lipid nanoparticle compositions and/or pharmaceutical compositions by storing the nanoparticle compositions and/or pharmaceutical compositions at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.). For example, the nanoparticle compositions and/or pharmaceutical compositions disclosed herein are stable for about at least 1 week, at least 2 weeks, at least 3 weeks, at least 4 weeks, at least 5 weeks, at least 6 weeks, at least 1 month, at least 2 months, at least 4 months, at least 6 months, at least 8 months, at least 10 months, at least 12 months, at least 14 months, at least 16 months, at least 18 months, at least 20 months, at least 22 months, or at least 24 months, e.g., at a temperature of 4° C. or lower (e.g., between about 4° C. and −20° C.). In one embodiment, the formulation is stabilized for at least 4 weeks at about 4° C. In certain embodiments, the pharmaceutical composition of the disclosure comprises a nanoparticle composition disclosed herein and a pharmaceutically acceptable carrier selected from one or more of Tris, an acetate (e.g., sodium acetate), an citrate (e.g., sodium citrate), saline, PBS, and sucrose. In certain embodiments, the pharmaceutical composition of the disclosure has a pH value between about 7 and 8 (e.g., 6.8, 6.9, 7.0, 7.1, 7.2, 7.3, 7.4, 7.5, 7.6, 7.7, 7.8, 7.9 or 8.0, or between 7.5 and 8 or between 7 and 7.8). For example, a pharmaceutical composition of the disclosure comprises a nanoparticle composition disclosed herein, Tris, saline and sucrose, and has a pH of about 7.5-8, which is suitable for storage and/or shipment at, for example, about −20° C. For example, a pharmaceutical composition of the disclosure comprises a nanoparticle composition disclosed herein and PBS and has a pH of about 7-7.8, suitable for storage and/or shipment at, for example, about 4° C. or lower. “Stability,” “stabilized,” and “stable” in the context of the present disclosure refers to the resistance of nanoparticle compositions and/or pharmaceutical compositions disclosed herein to chemical or physical changes (e.g., degradation, particle size change, aggregation, change in encapsulation, etc.) under given manufacturing, preparation, transportation, storage and/or in-use conditions, e.g., when stress is applied such as shear force, freeze/thaw stress, etc.

Nanoparticle compositions and/or pharmaceutical compositions including one or more nanoparticle compositions can be administered to any patient or subject, including those patients or subjects that can benefit from a therapeutic effect provided by the delivery of a therapeutic and/or prophylactic agent to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, horses, sheep, cats, dogs, mice, and/or rats.

A pharmaceutical composition including one or more nanoparticle compositions can be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure can be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions can be prepared in a variety of forms suitable for a variety of routes and methods of administration. For example, pharmaceutical compositions can be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms can comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutic and/or prophylactic agents, additional agents such as wetting agents, emulsifying and suspending agents, sweetening, flavoring, and/or perfuming agents. In certain embodiments for parenteral administration, compositions are mixed with solubilizing agents such as Cremophor™, alcohols, oils, modified oils, glycols, polysorbates, cyclodextrins, polymers, and/or combinations thereof.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

The disclosure features methods of delivering a therapeutic and/or prophylactic agent to a mammalian cell or organ, producing a polypeptide of interest in a mammalian cell, and treating a disease or disorder in a mammal in need thereof comprising administering to a mammal and/or contacting a mammalian cell with a nanoparticle composition including a therapeutic and/or prophylactic agent.

### 7. EXAMPLES

The examples in this section are offered by way of illustration, and not by way of limitation.

### Example 1: Preparation of Lipid Nanoparticle Formulations

The LNP formulations were prepared by mixing lipid and mRNA stock solution through a T-connector in 1:3 flow rate with total flow at 12 mL/min. The lipid stock solution was prepared in ethanol with total lipid concentration at 12.5 mM. The mRNA stock solution was prepared in 20 mM pH 4.0 citrate buffer with final concentration at 0.125 mg/mL. The size and distribution of all formulations were determined by Dynamic Light Scattering (DLS) measurement. The encapsulation efficiency (EE %) of each formulation were determined by Ribogreen assay following the vendor instructions from the kit. The concentration of formulation was determined using HPLC by measuring the UV absorption at 260 nm and comparing the standard concentration linear curve.

### Example 2: Physical Properties of Lipid Nanoparticle Formulations

Lipid nanoparticle compositions comprising mRNA and variable lipid compositions are listed in the following Tables 1 to 4 and 9. The physical characterization of LNP compositions were listed in the following Tables 5 to 8 and 10.

The structures and names of cationic lipids tested are listed below:

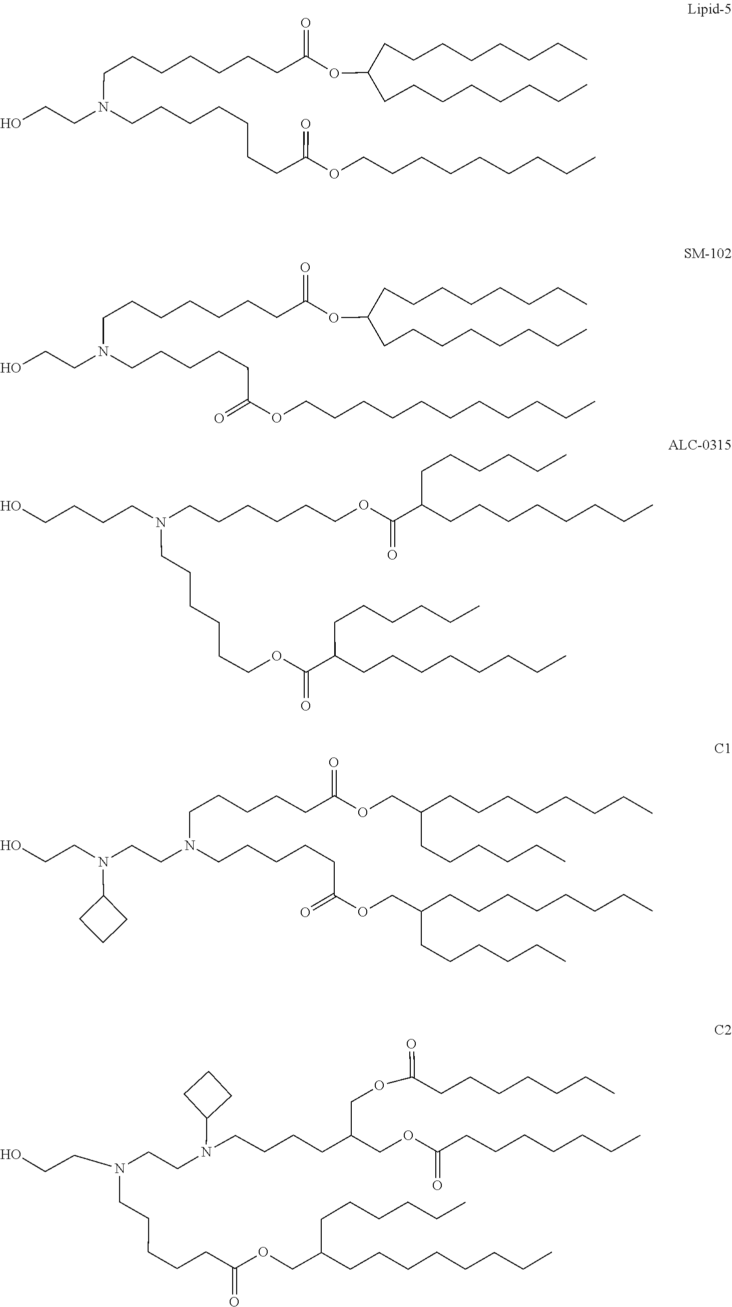

TABLE 1
LNPs comprising Lipid-5

| | Lipid (mol %) | | | |
|---|---|---|---|---|
| Formulation | Lipid-5/ | Cholesterol/ | DMG-PEG2000/ | Phospholipid/ |
| ID | % | % | % | % |
| L-0 | 50 | 38.5 | 1.5 | 10 |
| L-40/1.0 | 40 | 59.0 | 1.0 | 0 |
| L-40/1.5 | 40 | 58.5 | 1.5 | 0 |
| L-45/1.0 | 45 | 54.0 | 1.0 | 0 |
| L-45/1.5 | 45 | 53.5 | 1.5 | 0 |
| L-45/2.0 | 45 | 53.0 | 2.0 | 0 |
| L-47.5/1.0 | 47.5 | 51.5 | 1.0 | 0 |
| L-47.5/1.5 | 47.5 | 51.0 | 1.5 | 0 |
| L-47.5/2.0 | 47.5 | 50.5 | 2.0 | 0 |
| L-50/0.6 | 50 | 49.4 | 0.6 | 0 |
| L-50/0.8 | 50 | 49.2 | 0.8 | 0 |
| L-50/1.0 | 50 | 49.0 | 1.0 | 0 |
| L-50/1.5 | 50 | 48.5 | 1.5 | 0 |
| L-50/2.0 | 50 | 48.0 | 2.0 | 0 |
| L-50/2.5 | 50 | 47.5 | 2.5 | 0 |
| L-55/0.6 | 55 | 44.4 | 0.6 | 0 |
| L-55/0.8 | 55 | 44.2 | 0.8 | 0 |
| L-55/1.0 | 55 | 44.0 | 1.0 | 0 |
| L-55/1.5 | 55 | 43.5 | 1.5 | 0 |
| L-55/2.0 | 55 | 43.0 | 2.0 | 0 |
| L-55/2.5 | 55 | 42.5 | 2.5 | 0 |
| L-60/0.6 | 60 | 39.4 | 0.6 | 0 |
| L-60/0.8 | 60 | 39.2 | 0.8 | 0 |
| L-60/1.0 | 60 | 39.0 | 1.0 | 0 |
| L-60/1.5 | 60 | 38.5 | 1.5 | 0 |
| L-60/2.0 | 60 | 38.0 | 2.0 | 0 |
| L-60/2.5 | 60 | 37.5 | 2.5 | 0 |
| L-60/2.8 | 60 | 37.2 | 2.8 | 0 |
| L-60/3.0 | 60 | 37.0 | 3.0 | 0 |
| L-60/3.5 | 60 | 36.5 | 3.5 | 0 |
| L-65/1.0 | 65 | 34.0 | 1.0 | 0 |
| L-65/1.5 | 65 | 33.5 | 1.5 | 0 |
| L-65/2.0 | 65 | 33.0 | 2.0 | 0 |
| L-65/2.5 | 65 | 32.5 | 2.5 | 0 |
| L-67.5/1.5 | 67.5 | 31 | 1.5 | 0 |
| L-67.5/2.0 | 67.5 | 30.5 | 2.0 | 0 |
| L-70/1.5 | 70 | 28.5 | 1.5 | 0 |
| L-70/2.5 | 70 | 27.5 | 2.5 | 0 |

TABLE 2
LNPs comprising SM-102

| | Lipid (mol %) | | | |
|---|---|---|---|---|
| Formulation | SM-102/ | Cholesterol/ | DMG- | Phospholipid/ |
| ID | % | % | PEG2000/% | % |
| S-0 | 50 | 38.5 | 1.5 | 10 |
| S-40/1.5 | 40 | 58.5 | 1.5 | 0 |
| S-42.5/1.5 | 42.5 | 56.0 | 1.5 | 0 |
| S-45/1.0 | 45 | 54.0 | 1.0 | 0 |
| S-45/1.5 | 45 | 53.5 | 1.5 | 0 |
| S-45/2.0 | 45 | 53.0 | 2.0 | 0 |
| S-47.5/0.6 | 47.5 | 51.9 | 0.6 | 0 |
| S-47.5/0.8 | 47.5 | 51.7 | 0.8 | 0 |
| S-47.5/1.0 | 47.5 | 51.5 | 1.0 | 0 |
| S-47.5/2.0 | 47.5 | 50.5 | 2.0 | 0 |
| S-50/0.6 | 50 | 49.4 | 0.6 | 0 |
| S-50/0.8 | 50 | 49.2 | 0.8 | 0 |
| S-50/1.0 | 50 | 49.0 | 1.0 | 0 |
| S-50/1.5 | 50 | 48.5 | 1.5 | 0 |
| S-50/2.0 | 50 | 48.0 | 2.0 | 0 |
| S-50/2.5 | 50 | 47.5 | 2.5 | 0 |
| S-55/0.8 | 55 | 44.2 | 0.8 | 0 |
| S-55/1.0 | 55 | 44.0 | 1.0 | 0 |
| S-55/1.5 | 55 | 43.5 | 1.5 | 0 |
| S-55/2.0 | 55 | 43.0 | 2.0 | 0 |
| S-55/2.5 | 55 | 42.5 | 2.5 | 0 |
| S-55/2.7 | 55 | 42.3 | 2.7 | 0 |
| S-60/1.0 | 60 | 39.0 | 1.0 | 0 |
| S-60/1.5 | 60 | 38.5 | 1.5 | 0 |
| S-60/2.0 | 60 | 38.0 | 2.0 | 0 |
| S-60/2.5 | 60 | 37.5 | 2.5 | 0 |
| S-60/2.7 | 60 | 37.3 | 2.7 | 0 |
| S-60/3.0 | 60 | 37.0 | 3.0 | 0 |
| S-65/1.0 | 65 | 34.0 | 1.0 | 0 |
| S-65/1.5 | 65 | 33.5 | 1.5 | 0 |
| S-65/2.0 | 65 | 33.0 | 2.0 | 0 |
| S-65/2.5 | 65 | 32.5 | 2.5 | 0 |
| S-67.5/1.5 | 67.5 | 31 | 1.5 | 0 |
| S-67.5/2.0 | 67.5 | 30.5 | 2.0 | 0 |
| S-70/1.5 | 70 | 28.5 | 1.5 | 0 |

TABLE 3
LNPs comprising ALC-0315

| | Lipid (mol %) | | | |
|---|---|---|---|---|
| Formulation | ALC-0315/ | Cholesterol/ | DMG- | Phospholipid/ |
| ID | % | % | PEG2000/% | % |
| A-0 | 50 | 38.5 | 1.5 | 10 |
| A-55/1.5 | 55 | 43.5 | 1.5 | 0 |
| A-55/2.0 | 55 | 43.0 | 2.0 | 0 |
| A-55/2.5 | 55 | 42.5 | 2.5 | 0 |
| A-60/1.0 | 60 | 39 | 1.0 | 0 |
| A-60/1.5 | 60 | 38.5 | 1.5 | 0 |
| A-60/2.0 | 60 | 38.0 | 2.0 | 0 |
| A-60/2.5 | 60 | 37.5 | 2.5 | 0 |
| A-65/1.0 | 65 | 34.0 | 1.0 | 0 |
| A-65/1.5 | 65 | 33.5 | 1.5 | 0 |
| A-65/2.0 | 65 | 33.0 | 2.0 | 0 |

TABLE 4
LNPs comprising lipid C1

| | Lipid (mol %) | | | |
|---|---|---|---|---|
| Formulation | C1/ | Cholesterol/ | DMG-PEG2000/ | Phospholipid/ |
| ID | % | % | % | % |
| C1-0 | 50 | 38.5 | 1.5 | 10 |
| C1-60/1.0 | 60 | 39.0 | 1.0 | 0 |
| C1-60/1.5 | 60 | 38.5 | 1.5 | 0 |
| C1-65/1.0 | 65 | 34.0 | 1.0 | 0 |
| C1-65/1.5 | 65 | 33.5 | 1.5 | 0 |
| C1-65/2.0 | 65 | 33.0 | 2.0 | 0 |
| C1-65/2.5 | 65 | 32.5 | 2.5 | 0 |

TABLE 5
Physicochemical characterization of LNPs comprising Lipid-5

| Formulation ID | Size (nm) | PDI | EE % |
|---|---|---|---|
| L-0 | 69.1 | 0.040 | 92.1 |
| L-40/1.0 | 133.0 | 0.443 | 77.8 |
| L-40/1.5 | 124.2 | 0.252 | 79.9 |
| L-45/1.0 | 139.7 | 0.281 | 76.1 |
| L-45/1.5 | 111.4 | 0.232 | 89.9 |
| L-45/2.0 | 73.3 | 0.194 | 91.6 |
| L-47.5/1.0 | 144.3 | 0.258 | 81.2 |
| L-47.5/1.5 | 105.7 | 0.190 | 90.8 |
| L-47.5/2.0 | 79.6 | 0.161 | 92.1 |
| L-50/0.6 | 155.2 | 0.185 | 78.3 |
| L-50/0.8 | 150.2 | 0.244 | 76.9 |
| L-50/1.0 | 114.6 | 0.087 | 87.3 |
| L-50/1.5 | 86.7 | 0.118 | 91.6 |
| L-50/2.0 | 70.5 | 0.052 | 89.4 |
| L-50/2.5 | 58.7 | 0.051 | 92.1 |
| L-55/0.6 | 177.3 | 0.057 | 91.0 |
| L-55/0.8 | 141.2 | 0.052 | 90.6 |
| L-55/1.0 | 119.0 | 0.073 | 91.9 |
| L-55/1.5 | 89.8 | 0.042 | 91.6 |
| L-55/2.0 | 79.2 | 0.024 | 89.9 |
| L-55/2.5 | 64.7 | 0.042 | 91.9 |
| L-60/0.6 | 182.7 | 0.016 | 91.6 |
| L-60/0.8 | 154.3 | 0.007 | 91.3 |
| L-60/1.0 | 131.5 | 0.045 | 91.7 |
| L-60/1.5 | 107.2 | 0.040 | 89.1 |
| L-60/2.0 | 93.5 | 0.010 | 89.0 |
| L-60/2.5 | 86.1 | 0.050 | 90.1 |
| L-60/2.8 | 69.9 | 0.036 | 87.5 |
| L-60/3.0 | 67.5 | 0.034 | 91.6 |
| L-60/3.5 | 62.3 | 0.053 | 89.5 |
| L-65/1.0 | 150.8 | 0.022 | 89.6 |
| L-65/1.5 | 125.7 | 0.002 | 85.6 |
| L-65/2.0 | 102.4 | 0.015 | 85.0 |
| L-65/2.5 | 85.0 | 0.045 | 89.2 |
| L-67.5/1.5 | 122.0 | 0.002 | 88.6 |
| L-67.5/2.0 | 107.6 | 0.095 | 90.4 |
| L-70/1.5 | 132.5 | 0.017 | 71.2 |
| L-70/2.5 | 94.3 | 0.037 | 83.4 |

TABLE 6
Physicochemical characterization of LNPs comprising SM-102

| Formulation ID | Size (nm) | PDI | EE % |
|---|---|---|---|
| S-0 | 68.8 | 0.016 | 92.1 |
| S-42.5/1.5 | 81.0 | 0.101 | 92.0 |
| S-45/1.5 | 112.9 | 0.162 | 90.5 |
| S-45/2.0 | 72.1 | 0.054 | 91.6 |
| S-47.5/0.6 | 165.8 | 0.004 | 93.2 |
| S-47.5/0.8 | 149.0 | 0.019 | 92.5 |
| S-47.5/1.0 | 108.1 | 0.049 | 91.9 |
| S-47.5/2.0 | 77.4 | 0.082 | 91.2 |
| S-50/0.6 | 181.4 | 0.032 | 92.1 |
| S-50/0.8 | 124.3 | 0.063 | 92.8 |
| S-50/1.0 | 124.4 | 0.106 | 90.8 |
| S-50/1.5 | 93.5 | 0.035 | 91.6 |
| S-50/2.0 | 79.5 | 0.017 | 92.5 |
| S-50/2.5 | 70.9 | 0.019 | 89.6 |
| S-55/0.6 | 201.6 | 0.031 | 90.8 |
| S-55/0.8 | 167.6 | 0.002 | 91.3 |
| S-55/1.0 | 134.2 | 0.021 | 92.8 |
| S-55/1.5 | 105.3 | 0.002 | 92.1 |
| S-55/2.0 | 85.5 | 0.005 | 90.9 |
| S-55/2.5 | 68.4 | 0.032 | 91.7 |
| S-55/2.7 | 74.4 | 0.025 | 89.1 |
| S-60/1.0 | 142.8 | 0.008 | 90.8 |
| S-60/1.5 | 112.1 | 0.003 | 90.4 |
| S-60/2.0 | 90.5 | 0.031 | 91.7 |
| S-60/2.5 | 80.0 | 0.029 | 91.4 |
| S-60/2.7 | 83.6 | 0.033 | 85.0 |
| S-60/3.0 | 69.0 | 0.017 | 91.1 |
| S-65/1.0 | 153.8 | 0.022 | 89.2 |
| S-65/1.5 | 121.3 | 0.014 | 91.2 |
| S-65/2.0 | 107.7 | 0.024 | 90.3 |
| S-67.5/1.5 | 145.3 | 0.051 | 83.4 |
| S-67.5/2.0 | 110.4 | 0.052 | 77.6 |
| S-70/1.5 | 133.2 | 0.019 | 90.6 |

TABLE 7
Physicochemical characterization of LNPs comprising ALC-0315

| Formulation ID | Size (nm) | PDI | EE % |
|---|---|---|---|
| A-0 | 65.2 | 0.061 | 88.9 |
| A-55/1.5 | 87.81 | 0.091 | 85.90 |
| A-55/2.0 | 77.36 | 0.036 | 86.17 |
| A-55/2.5 | 75.68 | 0.006 | 87.78 |
| A-60/1.0 | 126.2 | 0.027 | 87.82 |
| A-60/1.5 | 103.7 | 0.008 | 86.50 |
| A-60/2.0 | 93.14 | 0.014 | 85.94 |
| A-60/2.5 | 84.05 | 0.022 | 83.99 |
| A-65/1.0 | 148.9 | 0.019 | 86.4 |
| A-65/1.5 | 129.1 | 0.019 | 81.8 |
| A-65/2.0 | 93.1 | 0.014 | 85.9 |

TABLE 8
Physicochemical characterization of LNPs comprising C1

| Formulation ID | Size (nm) | PDI | EE % |
|---|---|---|---|
| C1-0 | 68.9 | 0.041 | 92.39 |
| C1-60/1.0 | 131.1 | 0.02 | 91.39 |
| C1-60/1.5 | 101.9 | 0.05 | 89.44 |
| C1-65/1.0 | 149.2 | 0.026 | 91.95 |
| C1-65/1.5 | 135.9 | 0.021 | 87.26 |
| C1-65/2.0 | 115.3 | 0.034 | 87.13 |
| C1-65/2.5 | 98.9 | 0.003 | 88.03 |

TABLE 9
LNPs comprising lipid C2

| | Lipid (mol %) | | | |
|---|---|---|---|---|
| Formulation | C2/ | Cholesterol/ | DMG-PEG2000/ | Phospholipid/ |
| ID | % | % | % | % |
| C2-0 | 50 | 38.5 | 1.5 | 10 |
| C2-35/1.5 | 35 | 63.5 | 1.5 | 0 |
| C2-37.5/1.5 | 37.5 | 61 | 1.5 | 0 |
| C2-40/1.0 | 40 | 59 | 1 | 0 |
| C2-40/1.5 | 40 | 58.5 | 1.5 | 0 |
| C2-40/2.0 | 40 | 58 | 2 | 0 |
| C2-40/2.5 | 40 | 57.5 | 2.5 | 0 |
| C2-40/3.0 | 40 | 57 | 3 | 0 |
| C2-45/0.75 | 45 | 54.25 | 0.75 | 0 |
| C2-45/1.0 | 45 | 54 | 1 | 0 |
| C2-45/1.5 | 45 | 53.5 | 1.5 | 0 |
| C2-45/2.0 | 45 | 53 | 2 | 0 |
| C2-45/2.5 | 45 | 52.5 | 2.5 | 0 |
| C2-45/3.0 | 45 | 52 | 3 | 0 |
| C2-45/3.5 | 45 | 51.5 | 3.5 | 0 |
| C2-47.5/0.75 | 47.5 | 51.75 | 0.75 | 0 |
| C2-47.5/1.0 | 47.5 | 51.5 | 1 | 0 |
| C2-47.5/1.5 | 47.5 | 51 | 1.5 | 0 |
| C2-47.5/2.0 | 47.5 | 50.5 | 2 | 0 |
| C2-47.5/2.5 | 47.5 | 50 | 2.5 | 0 |
| C2-47.5/3.0 | 47.5 | 49.5 | 3 | 0 |
| C2-50/0.5 | 50 | 49.5 | 0.5 | 0 |
| C2-50/0.75 | 50 | 49.25 | 0.75 | 0 |
| C2-50/1.0 | 50 | 49 | 1 | 0 |
| C2-50/1.5 | 50 | 48.5 | 1.5 | 0 |
| C2-50/2.0 | 50 | 48 | 2 | 0 |
| C2-50/2.5 | 50 | 47.5 | 2.5 | 0 |
| C2-55/1.0 | 55 | 44 | 1 | 0 |
| C2-55/1.5 | 55 | 43.5 | 1.5 | 0 |
| C2-55/2.0 | 55 | 43 | 2 | 0 |
| C2-55/2.5 | 55 | 42.5 | 2.5 | 0 |
| C2-60/0.5 | 60 | 39.5 | 0.5 | 0 |
| C2-60/1.0 | 60 | 39 | 1 | 0 |
| C2-60/1.5 | 60 | 38.5 | 1.5 | 0 |
| C2-60/2.0 | 60 | 38 | 2 | 0 |
| C2-60/2.5 | 60 | 37.5 | 2.5 | 0 |
| C2-65/0.5 | 65 | 34.5 | 0.5 | 0 |
| C2-65/1.0 | 65 | 34 | 1 | 0 |
| C2-65/1.5 | 65 | 33.5 | 1.5 | 0 |
| C2-65/2.0 | 65 | 33 | 2 | 0 |
| C2-65/2.5 | 65 | 32.5 | 2.5 | 0 |

TABLE 10
Physicochemical characterization of LNPs comprising C2

| Formulation ID | Size (nm) | PDI | EE % |
|---|---|---|---|
| C2-0 | 80.7 | 0.03 | 96.4 |
| C2-35/1.5 | 148.5 | 0.27 | 77.9 |
| C2-37.5/1.5 | 130.2 | 0.27 | 85.3 |
| C2-40/1.0 | 124.2 | 0.01 | 91.5 |
| C2-40/1.5 | 95.0 | 0.06 | 91.9 |
| C2-40/2.0 | 78.2 | 0.09 | 94.3 |
| C2-40/2.5 | 71.6 | 0.06 | 92.4 |
| C2-40/3.0 | 60.6 | 0.07 | 92.8 |
| C2-45/0.75 | 160.2 | 0.11 | 77.5 |
| C2-45/1.0 | 130.4 | 0.02 | 93.4 |
| C2-45/1.5 | 103.5 | 0.06 | 92.5 |
| C2-45/2.0 | 88.8 | 0.04 | 91.7 |
| C2-45/2.5 | 79.6 | 0.04 | 91.6 |
| C2-45/3.0 | 68.1 | 0.06 | 92.3 |
| C2-45/3.5 | 61.2 | 0.07 | 89.3 |
| C2-47.5/0.75 | 164.9 | 0.09 | 82.0 |
| C2-47.5/1.0 | 135.7 | 0.05 | 90.5 |
| C2-47.5/1.5 | 110.9 | 0.11 | 93.4 |
| C2-47.5/2.0 | 95.6 | 0.06 | 93.2 |
| C2-47.5/2.5 | 81.51 | 0.02 | 91.5 |
| C2-47.5/3.0 | 72.5 | 0.09 | 90.1 |
| C2-50/0.5 | 188.7 | 0.05 | 77.9 |
| C2-50/0.75 | 168.8 | 0.08 | 85.0 |
| C2-50/1.0 | 142.1 | 0.02 | 94.6 |
| C2-50/1.5 | 124.3 | 0.01 | 93.0 |
| C2-50/2.0 | 104.5 | 0.02 | 92.0 |
| C2-50/2.5 | 81.1 | 0.02 | 94.7 |
| C2-55/1.0 | 156.1 | 0.00 | 94.4 |
| C2-55/1.5 | 129.1 | 0.11 | 92.4 |
| C2-55/2.0 | 114.2 | 0.01 | 91.6 |
| C2-55/2.5 | 91.4 | 0.03 | 93.0 |
| C2-60/0.5 | 235.8 | 0.02 | 82.1 |
| C2-60/1.0 | 173.6 | 0.00 | 91.8 |
| C2-60/1.5 | 140.1 | 0.01 | 90.8 |
| C2-60/2.0 | 118.9 | 0.03 | 76.3 |
| C2-60/2.5 | 99.7 | 0.04 | 84.0 |
| C2-65/0.5 | 286.3 | 0.03 | 75.0 |
| C2-65/1.0 | 211.6 | 0.03 | 82.7 |
| C2-65/1.5 | 160.0 | 0.01 | 82.1 |
| C2-65/2.0 | 133.0 | 0.07 | 69.0 |
| C2-65/2.5 | 111.6 | 0.01 | 53.6 |

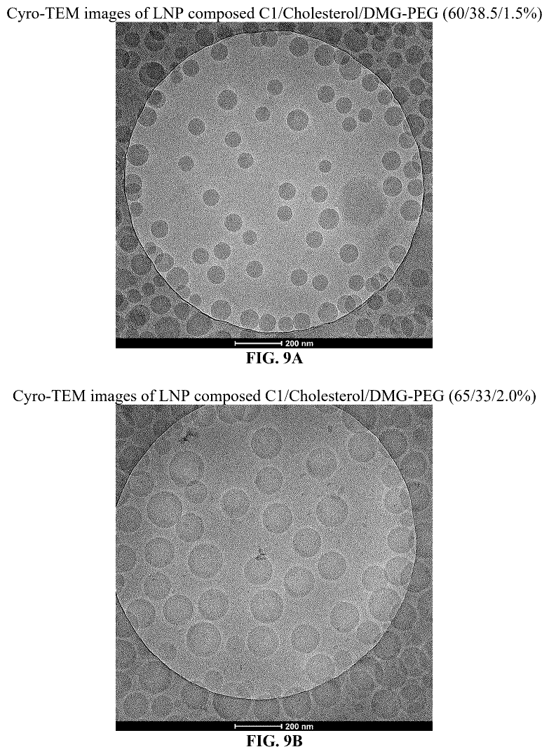

Cyro-EM images of LNP composed Cl/Cholesterol/DMG-PEG (60/38.5/1.5% o) and LNP composed C1/Cholesterol/DMG-PEG (65/33/2.0%) are provided in FIG. 9A and FIG. 9B, respectively. The 3-components LNP samples were concentrated 20 times (Volume change) by centrifugation using Amicon® Ultra-4. Samples (3 l) were deposited on a holey carbon grid that was glow-discharged (Quantifoil R1.2/1.3) and vitrificated using a Vitrobot Mark IV (Thermo Fisher Scientific) instrument, separately. Cryo-EM imaging was conducted on a Talos F200C Equipped with a Ceta 4 k×4 k camera, operated at 200 kV accelerating voltage. The results show that Cl based 3-componants LNP exhibit homogeneous morphologies of solid spheres that lack an aqueous core, which is different from multiple laminar phase solid nanoparticle or conventional liposome.

### Example 3: In Vivo Protein Expression

Lipid nanoparticles with different compositions (listed in Tables 1-4) encapsulating human erythropoietin (hEPO) mRNA were systemically administered to 6-8-week-old female ICR mice at 0.5 mg/kg dose weekly by tail vein injection and mice blood were sampled at specific time points (e.g. 6 hours) post administration. For the same formulation, three to five mice were included for the administration. Mice were euthanized by CO₂ overdose after the last sampling time point. Serum was separated from total blood by centrifugation at 5000 g for 10 minutes at 4° C., snap-frozen and stored at −80° C. for analysis. To evaluate hEPO expression, ELISA assays were carried out using a commercial kit (DEP00, R&D systems;) according to manufacturer's instructions.

GFP encoded mRNA was encapsulated in Lipid-5 containing LNP with different lipid compositions. There LNP were systemically administered to 8-10-week-old female Balb/c mice at 0.1 mg/kg dose by intramuscular injection. The muscle around the injection point was collected 6 hours after i.m. administration, following by cell lysis with Cell Lysis Buffer. The total protein from the samples have been detected using Pierce BCA Protein Assay Kit. The GFP and IL-6 in muscle have been determined by ELISA assay with GFP SimpleStem ELISA Kit and Mouse IL-6 ELISA Kit, respectively.

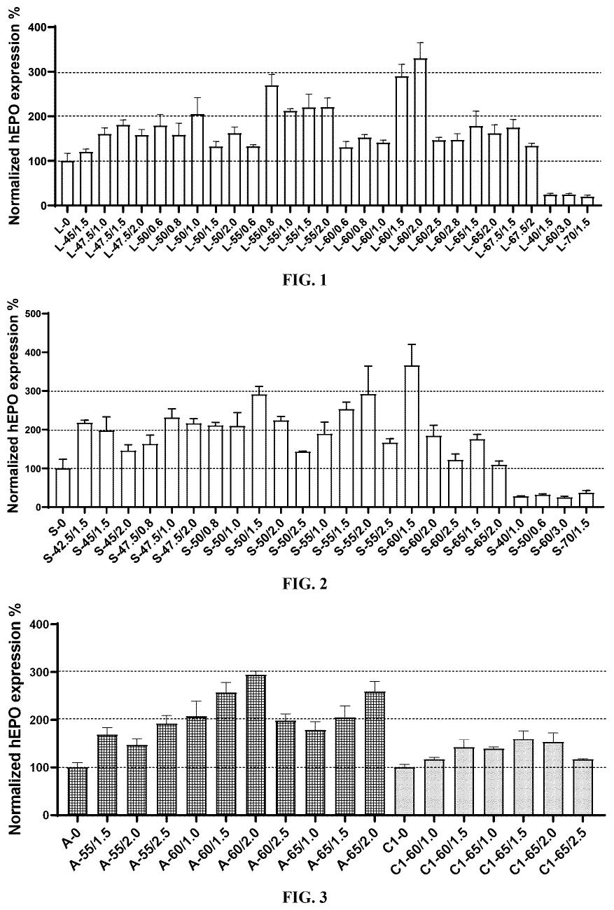

FIG. 1 displays the normalized hEPO expression levels when the three-lipid LNPs comprising Lipid-5 are administrated in vivo. The Lipid-5 formulation group also shows impressive in vivo hEPO expression. The conventional four-lipid LNP (L-0) was used as the reference group to normalize the hEPO expression level. In the Lipid-5 formulation group, 25 different LNP formulations were found to have higher in vivo hEPO expression level than the conventional formulation (L-0). Among these formulations, the amount of cationic lipid (Lipid-5) ranges from about 40 mol % to about 70 mol %, and the amount of the PEG-lipid ranges from about 0.5 mol % to about 3.0 mol %. Moreover, it was also observed that 7 of these formulations increased in vivo gene expression level by more than 2-fold compared with the L-0 control. The data also shows that the three-lipid LNP formulations containing Lipid-5 in the amount below 40 mol % or above 70 mol % and the PEG-lipid above 3 mol % have relatively low in vivo activity.

FIG. 2 displays the normalized hEPO expression levels when the three-lipid LNPs comprising SM-102 are administrated in vivo. The SM-102 formulation group also shows apparent benefits in terms of in vivo hEPO expression when compared with the reference formulation (S-0). In the SM-102 formulation group, 20 of the tested LNP formulations were found to have higher in vivo hEPO expression level than the conventional formulation (S-0). Among these formulations, the amount of cationic lipid (SM-102) ranges from about 40 mol % to about 70 mol %, and the amount of the PEG-lipid ranges from about 0.5 mol % to about 3 mol %. It was also observed that 10 of these formulations increased in vivo gene expression level by more than 2-fold compared with the S-0 control. Moreover, one of the formulations showed over three times in vivo hEPO expression level when compared with the reference formulation (S-0). The data also shows that the three-lipid LNP formulations containing SM-102 in the amount below 40 mol % or above 70 mol % and the PEG-lipid above 3 mol % have relatively low in vivo activity.

Based on the results, LNPs comprising cationic lipid in the amount from about 40 mol % to about 70 mol % and PEG-lipid in the amount below 3 mol % appears to be a general area in which the in vivo activities of three-lipid LNP formulations outperform the conventional four-lipid LNP formulations.

FIG. 3 displays the normalized hEPO expression levels when the three-lipid LNPs comprising cationic lipid ALC-0315 or C1 are administrated in vivo. In the ALC-0315 group, ten different LNP compositions have been prepared and characterized (Table 3 and Table 7). All of the 10 LNP formulations containing ALC-0315 were found to have higher in vivo hEPO expression level than the conventional formulation (A-0). It was also observed that 5 of these formulations increased in vivo gene expression level by more than 2-fold compared with the A-0 control. Additionally, 6 LNP formulations comprising C1 have been prepared and characterized (Table 4 and Table 8). These LNP formulations show higher in vivo activity than the conventional formulation (C1-0).

In U.S. Pat. No. 11,191,849, it mentioned that increasing PEG-lipid ratio up to 3.3% in PL-free (phospholipid free) formulations slightly affected the LNP efficiency in terms of ApoB silencing activity. However, data shown herein shows a very different story in terms of mRNA delivery when three-lipid LNPs comprising more than 3 mol % of PEG-lipid were used. FIG. 4 displays the normalized hEPO expression levels when the three-lipid LNPs comprising more than 3 mol % of PEG-lipid are administrated in vivo. All formulations comprising over 3 mol % of PEG-lipid showed very low in vivo hEPO expression compared with other three-lipid LNPs comprising lower levels of PEG-lipid. The hEPO expression level of all three-lipid LNPs comprising over 3 mol % of PEG-lipid is less than 50% than that of the conventional formulation.

Table 11 and FIG. 8 display the normalized hEPO expression levels when the three-lipid LNPs comprising C2 are administrated in vivo. The C2 containing three-lipids LNPs showed good in vivo hEPO expression activity. Seventeen of the tested LNP formulations were found to have higher hEPO expression level than conventional formulation (C2-0). The cationic lipid in the formulation ranges from about 40% to about 55%, while the PEG-lipid ranges from about 1.0% to about 2.5%.

TABLE 11
hEPO expression of LNPs comprising C2

| Formulation ID | hEPO (μg/ml) | SEM (μg/ml) | Normalization/% |
|---|---|---|---|
| C2-0 | 11.4 | 0.9 | 100.0 |
| C2-40/1.0 | 18.4 | 2.5 | 160.7 |
| C2-40/1.5 | 11.7 | 0.6 | 102.0 |
| C2-45/1.0 | 18.0 | 2.9 | 157.8 |
| C2-45/1.5 | 19.5 | 4.1 | 170.7 |
| C2-45/2.0 | 14.2 | 0.8 | 123.9 |
| C2-45/2.5 | 14.4 | 2.0 | 126.3 |
| C2-47.5/1.0 | 16.3 | 1.0 | 143.0 |
| C2-47.5/1.5 | 17.6 | 1.0 | 153.6 |
| C2-47.5/2.0 | 16.7 | 1.7 | 146.1 |
| C2-50/1.0 | 16.3 | 0.9 | 142.9 |
| C2-50/1.5 | 20.1 | 3.4 | 176.0 |
| C2-50/2.0 | 19.1 | 0.7 | 167.0 |
| C2-50/2.5 | 11.6 | 1.4 | 101.3 |
| C2-55/1.0 | 12.2 | 1.8 | 107.0 |
| C2-55/1.5 | 21.5 | 0.6 | 188.5 |
| C2-55/2.0 | 22.9 | 1.1 | 200.2 |
| C2-60/1.5 | 11.5 | 0.5 | 100.3 |
| C2-50/0.75 | 4.3 | 0.7 | 37.2 |
| C2-50/3.0 | 7.0 | 0.8 | 61.2 |
| C2-65/1.5 | 1.9 | 0.4 | 16.5 |

### Example 4: Fragment Analysis

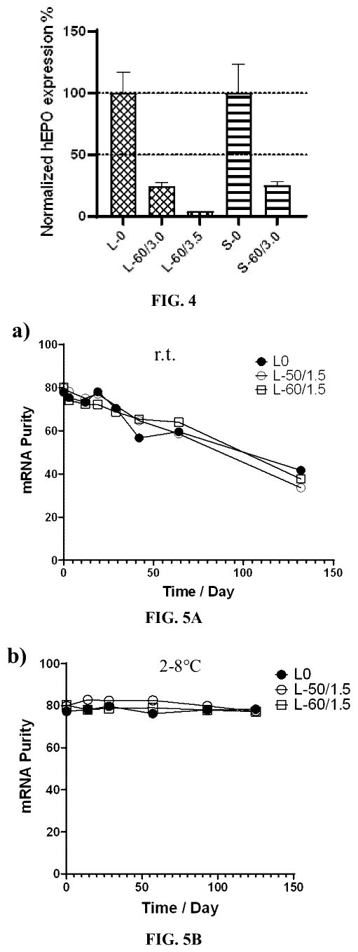

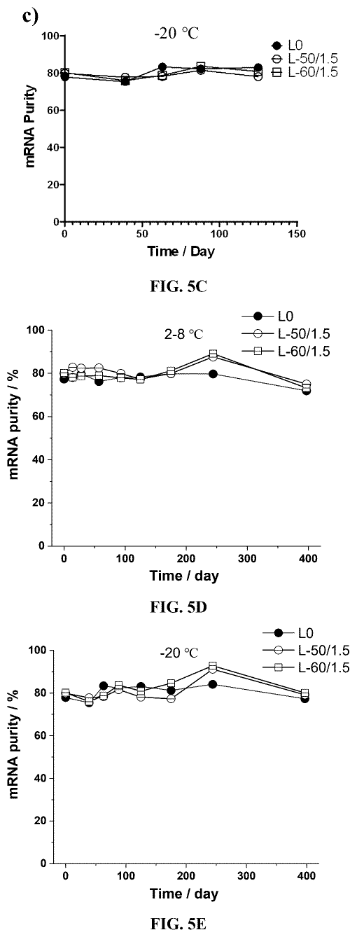

As shown in FIG. 5A, FIG. 51B, and FIG. 5C, fragment analysis (FA) was performed to test the long-term stability of the LNP formulations under different temperatures. The mRNA was isolated from the LNP using a Rneasy MiNi Kit(50). The Fragment Analysis was performed on Agilant 5280 Analysizer.

The LNP could protect the RNA from degradation by omnipresent RNases. Three LNPs comprising Lipid-5 encapsulating mRNA encoding GFP were used in fragment analysis. These formulations were stored at room temperature (r.t.), 2-8° C. or −20° C. for over four months. At room temperature, the mRNA purity decreased over time in all three formulations. No clear difference was found between the conventional and three-lipid LNP formulations. When these LNPs were stored at 2-8° C. or −20° C., there were no significant decrease in RNA purity even after four months of storage in all of the three formulations tested. These results show that the three-lipid LNP formulations have similar or equal property in protecting encapsulated RNA from degradation.

FIG. 5D and FIG. 5E show the updated long-term stability of the Lipid-5 containing LNPs stored at 2-8° C. or −20° C., respectively, for up to 400 days. No clear decline of mRNA purity in Lipid-5 containing LNPs was found for both storage condition over a year.

### Example 5: Intramuscular Administration

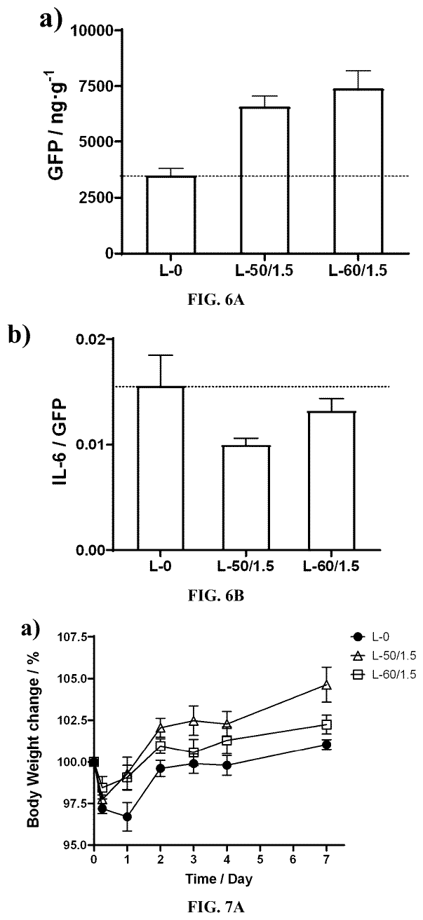

The intramuscular administration has been investigated with two LNPs (L-50/1.5, L-60/1.5) comprising Lipid 5 by comparing GFP expression level with conventional formulation (L-0). As shown in FIG. 6A and FIG. 6B, in the group intramuscularly administrated with three-lipid LNPs (L-50/1.5, L-60/1.5), there is about 2-fold increase in GFP expression level compared with the conventional formulation (L-0). The data shows that the three-lipid LNP formulations have significant advantage in gene expression through either intramuscular or intravenous administration. The cytokine interleukin-6 (IL-6) were detected to evaluate the effect of LNP administration to the immune simulation. When the IL-6 production was normalized to GFP expression, it was found that the three-lipid LNP formulations induced relative less IL-6 production in vivo at the same expression level of GFP, indicating that the three-lipid LNP formulations (L-50/1.5 and L-60/1.5) have relatively less side effect than the conventional four-lipid LNP formulation (L-0).

### Example 6: Body Weight Change

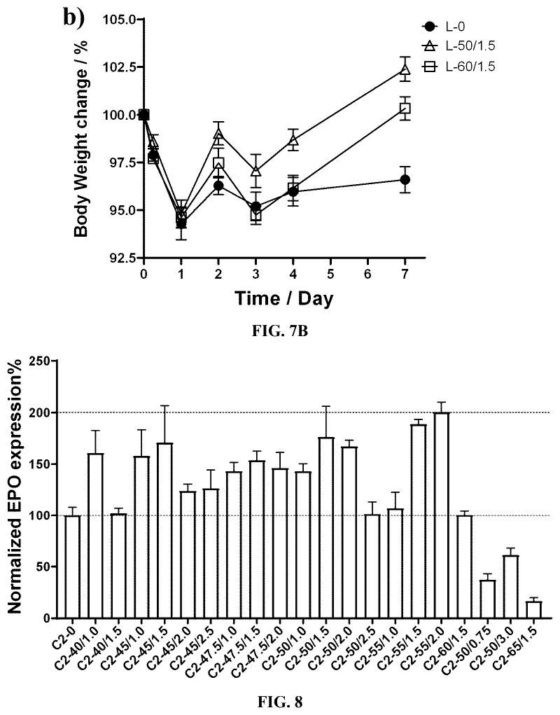

After administration, some side effects can be induced in mice due to the immune simulation of LNP, such as body weight change. Different LNP compositions containing Lipid-5 have been used to study the level and duration of side effects after intravenous or intramuscular administration. All LNP formulations encapsulated the mRNA encoding luciferase. Following administration of the LNPs, the changes of mice body weights were tracked over the course of 7 days (FIG. 7A and FIG. 7B). For intravenous administration, the three-lipid LNP formulations group showed lower maximum body weight loss and quicker body weight return. The group of mice intravenously injected with three-lipid LNP formulations (L-50/1.5 and L-60/1.5) returned to 100% body weight within two days, while the group intravenously injected with the conventional four-lipid LNP formulation (L-0) took more than four days to recover. The L-50/1.5 group showed comparably faster body weight increase, indicative of minimum side effect after intravenous administration of the LNP.

For intramuscular administration, all the three groups quickly lost over 5% of body weight within one day of administration, indicating that intramuscular administration has more serious side effect than intravenous administration in this system. Similarly, the group of mice treated with the three-lipid LNP formulation (L-50/1.5 and L-60/1.5) took less time to return their body weight after intramuscular administration compared with the group treated with the conventional four-lipid LNP formulation (L-0). The L-50/1.5 and L-60/1.5 groups took seven days to fully recover to their original body weight while the body weight in L-0 group only change slightly. The mice in the L-50/1.5 group showed faster body weight increase than other two groups, indicating that relatively less side effect was induced by this formulation.

1. A lipid nanoparticle comprising:
(a) a cationic lipid at the amount of from about 40 mol % to about 75 mol % of the total lipid present in the nanoparticle, wherein the cationic lipid comprises (i) at least one tertiary amine or quaternary ammonium moiety, and (ii) at least one C₁₄-C₄₂ alkyl ester moiety;
(b) a steroid at the amount of from about 22 mol % to about 59.5 mol % of the total lipid present in the nanoparticle;
(c) a polymer conjugated lipid at the amount of from about 0.5 mol % to about 2.5 mol % of the total lipid present in the nanoparticle; and
(d) a nucleic acid;
provided that the lipid nanoparticle comprises no more than 0.5 mole percent phospholipid.

2. The lipid nanoparticle of claim 1, wherein the amount of the cationic lipid is from about 55 mol % to about 65 mol % of the total lipid present in the nanoparticle, or the amount of the cationic lipid is about 60 mol % of the total lipid present in the nanoparticle; or
wherein the amount of the polymer conjugated lipid is from about 1 mol % to about 2 mol % of the total lipid present in the nanoparticle, or the amount of the polymer conjugated lipid is about 1.5 mol % of the total lipid present in the nanoparticle; or
wherein the amount of the steroid is from about 33 mol % to about 44 mol % of the total lipid present in the nanoparticle; or
the lipid nanoparticle does not comprise phospholipid.

3. (canceled)

4. (canceled)

5. (canceled)

6. (canceled)

7. The lipid nanoparticle of claim 1, wherein
(1) the amount of the cationic lipid is from about 45 mol % to about 70 mol % of the total lipid present in the nanoparticle, and wherein the amount of the polymer conjugated lipid is from about 1 mol % to about 2.5 mol % of the total lipid present in the nanoparticle;
(2) the amount of the cationic lipid is from about 45 mol % to about 65 mol % of the total lipid present in the nanoparticle, and wherein the amount of the polymer conjugated lipid is from about 1 mol % to about 2.5 mol % of the total lipid present in the nanoparticle;
(3) the amount of the cationic lipid is from about 45 mol % to about 65 mol % of the total lipid present in the nanoparticle, and wherein the amount of the polymer conjugated lipid is from about 1 mol % to about 2 mol % of the total lipid present in the nanoparticle;
(4) the amount of the cationic lipid is from about 50 mol % to about 65 mol % of the total lipid present in the nanoparticle, and wherein the amount of the polymer conjugated lipid is from about 0.5 mol % to about 2.5 mol % of the total lipid present in the nanoparticle;
(5) the amount of the cationic lipid is from about 50 mol % to about 60 mol % of the total lipid present in the nanoparticle, and wherein the amount of the polymer conjugated lipid is from about 0.8 mol % to about 2 mol % of the total lipid present in the nanoparticle;
(6) the amount of the cationic lipid is from about 50 mol % to about 60 mol % of the total lipid present in the nanoparticle, and wherein the amount of the polymer conjugated lipid is from about 1 mol % to about 2 mol % of the total lipid present in the nanoparticle;
(7) the amount of the cationic lipid is from about 55 mol % to about 65 mol % of the total lipid present in the nanoparticle, and wherein the amount of the polymer conjugated lipid is from about 1 mol % to about 2.5 mol % of the total lipid present in the nanoparticle;
(8) the amount of the cationic lipid is from about 60 mol % to about 65 mol % of the total lipid present in the nanoparticle, and wherein the amount of the polymer conjugated lipid is from about 1 mol % to about 2 mol % of the total lipid present in the nanoparticle;
(9) the amount of the cationic lipid is from about 60 mol % to about 65 mol % of the total lipid present in the nanoparticle, and wherein the amount of the polymer conjugated lipid is from about 1.5 mol % to about 2.5 mol % of the total lipid present in the nanoparticle; or
(10) the amount of the cationic lipid is from about 40 mol % to about 55 mol % of the total lipid present in the nanoparticle, and the amount of the polymer conjugated lipid is from about 1 mol % to about 2.5 mol % of the total lipid present in the nanoparticle.

8. (canceled)

9. (canceled)

10. (canceled)

11. (canceled)

12. (canceled)

13. (canceled)

14. (canceled)

15. (canceled)

16. (canceled)

17. The lipid nanoparticle of claim 1, wherein the polymer conjugated lipid is a polyethylene glycol (PEG)-conjugated lipid, or
the polymer conjugated lipid is a PEG-diacylglycerol (PEG-DAG) conjugate, a PEG-dialkyloxypropyl (PEG-DAA) conjugate, a PEG-dimyristyloxypropyl (PEG-DMA) conjugate, a PEG-distearyloxypropyl (PEG-DSA) conjugate, or a mixture thereof, or
the polymer conjugated lipid is DMG-PEG2000 or DMPE-PEG2000;
or
wherein the steroid is glucocorticoid, mineralocorticoid, clobetasol, cholesterol or a cholesterol derivative;
or
wherein the cationic lipid comprises a moiety of 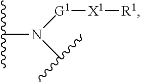
wherein G¹ is C₂-C₁₂ alkylene; X¹ is —C(═O)O— or —OC(═O)—; and R¹ is a branched C₁₄-C₄₂ alkyl.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The lipid nanoparticle of claim 1, wherein the cationic lipid is of Formula (I): 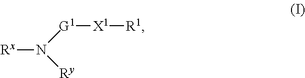
or a pharmaceutically acceptable salt, prodrug or stereoisomer thereof, wherein:
G¹ is C₂-C₁₂ alkylene;
X¹ is —C(═O)O— or —OC(═O)—;
R¹ is a branched C₁₄-C₄₂ alkyl;
R^{y} is C₁-C₆ alkyl or -G²-X²—R²;
G² is C₂-C₁₂ alkylene;
X² is —C(═O)O— or —OC(═O)—;
R² is a straight or branched C₆-C₄₂ alkyl;
R^{x} is C₁-C₆ alkyl or -G³-R³;
G³ is C₂-C₁₂ alkylene;
R³ is —N(R⁴)R⁵ or —OR⁶;
R⁴ is C₁-C₁₂ alkyl, C₃-C₈ cycloalkyl, C₃-C₈ cycloalkenyl, or C₆-C₁₀ aryl;
R⁵ is C₁-C₁₂ alkyl, C₃-C₈ cycloalkyl, C₃-C₈ cycloalkenyl, or C₆-C₁₀ aryl;
or R⁴ and R⁵ together with the nitrogen to which they are attached form a cyclic moiety;
R⁶ is hydrogen, C₁-C₁₂ alkyl, C₃-C₈ cycloalkyl, C₃-C₈ cycloalkenyl, or C₆-C₁₀ aryl; and
wherein each alkyl, alkenyl, cycloalkyl, cycloalkenyl, aryl, alkylene, and cyclic moiety is independently optionally substituted; or
the cationic lipid is of Formula (II): 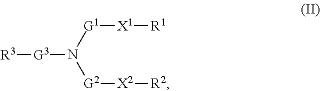
or a pharmaceutically acceptable salt, prodrug or stereoisomer thereof.

32. (canceled)

33. (canceled)

34. (canceled)

35. (canceled)

36. (canceled)

37. (canceled)

38. The lipid nanoparticle of claim 3104, wherein the cationic lipid is of Formula (II-A): 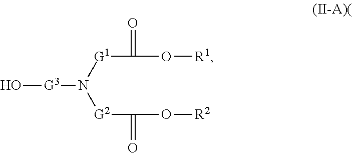
or a pharmaceutically acceptable salt, prodrug or stereoisomer thereof, wherein:
G¹ is C₅-C₇ alkylene;
R¹ is a branched C₁₄-C₁₉ alkyl;
G² is C₅-C₇ alkylene;
R² is a straight C₇-C₁₃ alkyl;
G³ is C₂-C₄ alkylene; or
the cationic lipid is of Formula (II-A-1): 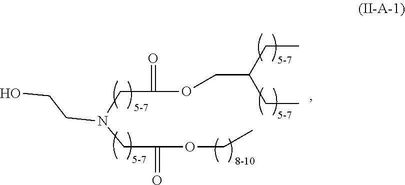
or a pharmaceutically acceptable salt, prodrug or stereoisomer thereof;
or
the cationic lipid is of Formula (II-B): 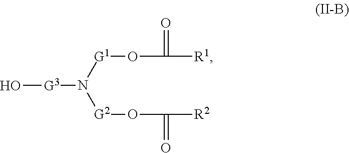
or a pharmaceutically acceptable salt, prodrug or stereoisomer thereof, wherein:
G¹ is C₅-C₇ alkylene;
R¹ is a branched C₁₄-C₁₉ alkyl;
G² is C₅-C₇ alkylene;
R² is a branched C₁₄-C₁₉ alkyl;
G³ is C₂-C₄ alkylene; or
the cationic lipid is of Formula (II-B-1): 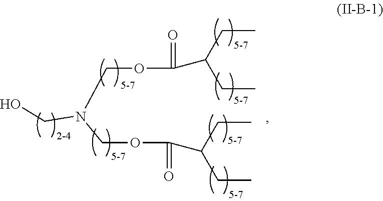
or a pharmaceutically acceptable salt, prodrug or stereoisomer thereof.

39. (canceled)

40. (canceled)

41. (canceled)

42. The lipid nanoparticle of claim 31, wherein the cationic lipid is of Formula (II-C): 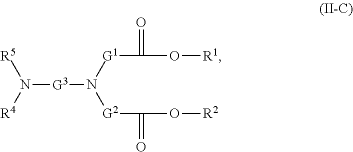
or a pharmaceutically acceptable salt, prodrug or stereoisomer thereof, wherein:
G¹ is C₅-C₇ alkylene;
R¹ is a branched C₁₄-C₁₉ alkyl;
G² is C₅-C₇ alkylene;
R² is a branched C₁₄-C₁₉ alkyl;
G³ is C₂-C₄ alkylene;
R⁴ is C₃-C₈ cycloalkyl; and
R⁵ is C₂-C₄ alkyl substituted with a hydroxy; or
the cationic lipid is of Formula (II-C-1): 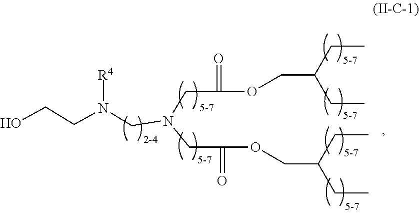
or a pharmaceutically acceptable salt, prodrug or stereoisomer thereof.

43. (canceled)

44. The lipid nanoparticle of claim 3, wherein the cationic lipid is

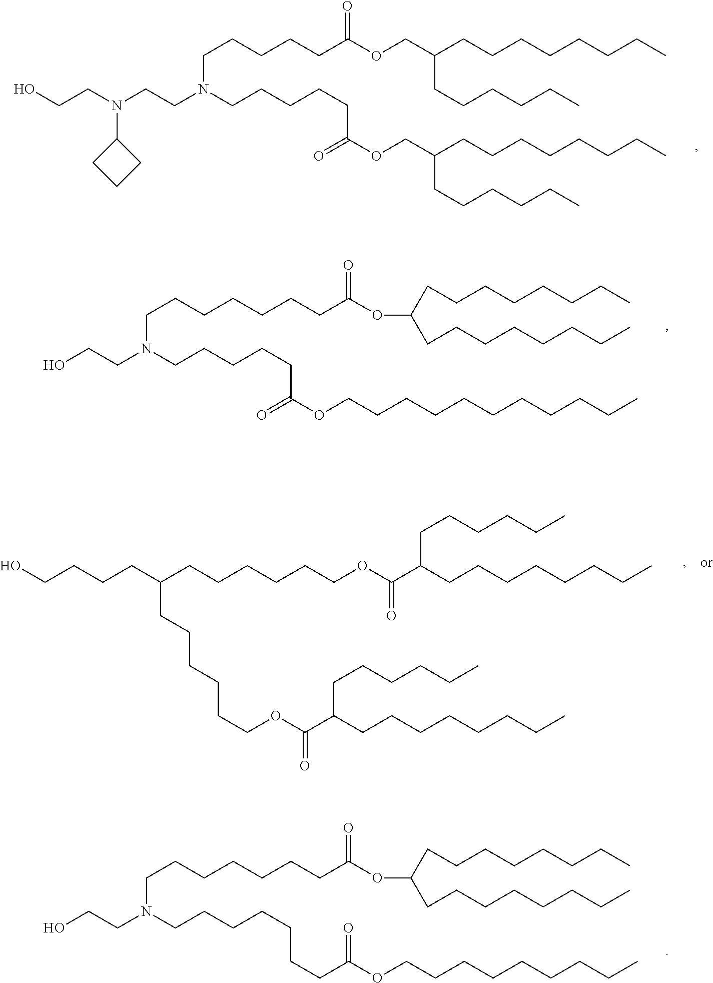 [SMILES: CCCCCCCCC(CCCCCC)COC(=O)CCCCCN(CCCCCC(=O)OCC(CCCCCC)CCCCCCCC)CCN(CCO)C1CCC1 ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCC(CCCCO)CCCCCCOC(=O)C(CCCCCC)CCCCCCCC ; CCCCCCCCCOC(=O)CCCCCCCN(CCO)CCCCCCCC(=O)OC(CCCCCCCC)CCCCCCCC]

45. The lipid nanoparticle of claim 1, wherein the cationic lipid is of Formula (III): 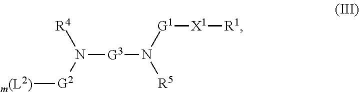
or a pharmaceutically acceptable salt, prodrug or stereoisomer thereof, wherein:
G¹ is C₂-C₁₂ alkylene;
X¹ is —C(═O)O— or —OC(═O)—;
R¹ is a branched C₁₄-C₄₂ alkyl;
G² is C₂-C₁₂ alkylene;
each L² is independently —X²—R²;
each X² is independently —C(═O)O— or —OC(═O)—;
each R² is independently a straight or branched C₆-C₄₂ alkyl;
m is 1 or 2;
G³ is C₂-C₁₂ alkylene;
R⁴ is C₁-C₁₂ alkyl, C₃-C₈ cycloalkyl, C₃-C₈ cycloalkenyl, or C₆-C₁₀ aryl;
R⁵ is C₁-C₁₂ alkyl, C₃-C₈ cycloalkyl, C₃-C₈ cycloalkenyl, or C₆-C₁₀ aryl; and
wherein each alkyl, alkenyl, cycloalkyl, cycloalkenyl, aryl, alkylene, and cyclic moiety is independently optionally substituted; or
the cationic lipid is of Formula (IV): 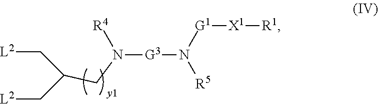
or a pharmaceutically acceptable salt, prodrug or stereoisomer thereof, wherein:
y1 is an integer from 0 to 9.

46. (canceled)

47. (canceled)

48. (canceled)

49. (canceled)

50. The lipid nanoparticle of claim 45, wherein the cationic lipid is of Formula (IV-A): 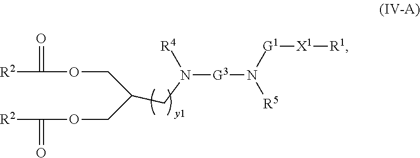
or a pharmaceutically acceptable salt, prodrug or stereoisomer thereof, wherein:
G¹ is C₅-C₇ alkylene;
R¹ is a branched C₁₄-C₁₉ alkyl;
each R² is independently a straight C₇-C₁₃ alkyl;
G³ is C₂-C₄ alkylene;
R⁴ is C₃-C₈ cycloalkyl;
R⁵ is C₂-C₄ alkyl substituted with a hydroxy; and
y1 is an integer from 2 to 6; or
the cationic lipid is of Formula (IV-A-1): 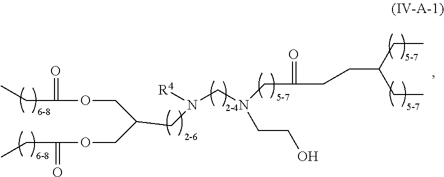
or a pharmaceutically acceptable salt, prodrug or stereoisomer thereof.

51. (canceled)

52. The lipid nanoparticle of claim 45, wherein the cationic lipid is

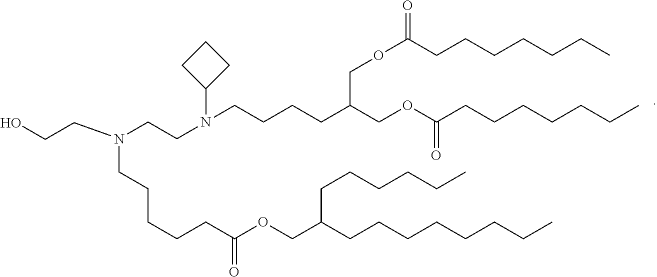 [SMILES: CCCCCCCCC(CCCCCC)COC(=O)CCCCCN(CCO)CCN(CCCCC(COC(=O)CCCCCCC)COC(=O)CCCCCCC)C1CCC1]

53. (canceled)

54. The lipid nanoparticle of claim 1, wherein the lipid nanoparticle has a lipid:nucleic acid mass ratio of from about 9:1 to about 20:1.

55. The lipid nanoparticle of claim 1, wherein the nucleic acid is an mRNA or a small interfering RNA (siRNA).

56. The lipid nanoparticle of claim 55, wherein the mRNA is an mRNA that encodes an antigen or a fragment or epitope thereof;
the mRNA is monocistronic mRNA or multicistronic mRNA; or
the mRNA is an mRNA that encodes an antigen or a fragment or epitope thereof, and the antigen is a pathogenic antigen or a tumor associated antigen.

57. (canceled)

58. (canceled)

59. (canceled)

60. (canceled)

61. (canceled)

62. The lipid nanoparticle of claim 1, wherein the nucleic acid is chemically modified.

63. The lipid nanoparticle of claim 1, wherein the size of the nanoparticle is from about 100 nm to about 140 nm;
the lipid nanoparticle has an encapsulation efficiency of at least about 80% of the nucleic acid; the size of the nanoparticle is increased as compared to a reference formulation; or
the nucleic acid encodes a RNA or protein; and wherein the amount of RNA or protein expressed from the nucleic acid in the nanoparticle in a mammalian cell or a tissue of a mammal is more than the amount of RNA or protein expressed from the nucleic acid in a reference formulation;
wherein the reference formulation differs from the lipid nanoparticle in that (i) the reference formulation comprises a phospholipid, and (ii) the molar ratio of the cationic lipid:the steroid:the phospholipid:the polymer conjugated lipid is about 50:38.5:10:1.5.

64. (canceled)

65. (canceled)

66. The lipid nanoparticle of claim 63, wherein
the size of the nanoparticle is from about 110 nm to about 130 nm;
the lipid nanoparticle has an encapsulation efficiency of at least about 90% of the nucleic acid;
the size of the nanoparticle is increased by at least about 50% as compared to the reference formulation;
the amount of RNA or protein expressed from the nucleic acid in the nanoparticle is at least about 50% more than the amount of RNA or protein expressed from the nucleic acid in a reference formulation; or
the amount of RNA or protein expressed from the nucleic acid in the nanoparticle is at least about 100% more than the amount of RNA or protein expressed from the nucleic acid in a reference formulation.

67. (canceled)

68. (canceled)

69. (canceled)

70. (canceled)

71. (canceled)

72. A pharmaceutical composition comprising the lipid nanoparticle of claim 1, and a pharmaceutically acceptable excipient.

73. (canceled)

74. (canceled)

75. (canceled)

76. A method for preventing or treating a disease or disorder in a human, comprising administering to the human a therapeutically effective amount of the lipid nanoparticle of claim 1.

77. (canceled)

78. (canceled)

79. The method of claim 76, wherein the administration is intranasal, intravenous, intraperitoneal, intramuscular, intraarticular, intralesional, intratracheal, subcutaneous, or intradermal administration.

80. (canceled)

81. (canceled)

82. (canceled)

83. (canceled)

84. (canceled)
